SEQUENCE LISTING (111 sequences)

SEQ ID NO: 1 - protein, 124 aa, Artificial Sequence
  VH of antibody 101E4
EVQLVESGGGLVQPKGSLKLSCAASGFSFNPYAMNWVRQGPGKGLEWVARIRSKSNNYATYYADSVKDRFTISRDDSENILYLQMNNLKTEDTAMYYCVRDYYGGRSSIGMDYWGHGTSVTVSS

SEQ ID NO: 2 - protein, 8 aa, Artificial Sequence
  VH-CDR1
GFSFNPYA

SEQ ID NO: 3 - protein, 10 aa, Artificial Sequence
  VH-CDR2
IRSKSNNYAT

SEQ ID NO: 4 - protein, 15 aa, Artificial Sequence
  VH-CDR3
VRDYYGGRSSIGMDY

SEQ ID NO: 5 - protein, 112 aa, Artificial Sequence
  VL of antibody 101E4
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCLQHLEYPFTFGGGTKLELK

SEQ ID NO: 6 - protein, 11 aa, Artificial Sequence
  VL-CDR1
KSLLHSNGNTY

SEQ ID NO: 7 - protein, 3 aa, Artificial Sequence
  VL-CDR2
RMS

SEQ ID NO: 8 - protein, 9 aa, Artificial Sequence
  VL-CDR3
LQHLEYPFT

SEQ ID NO: 9 - protein, 124 aa, Artificial Sequence
  VH of antibody 103G4
EVQLVESGGGLVRPKGSLKLSCAASGFSFNPYALNWVRQAPGKGLEWVARIRSKSNNYATYYADSVKDRFTISRDDSENILYLQMNNLKTEDTAMYYCVKDYYGTRPSIGMDYWGQGTSVTVSS

SEQ ID NO: 10 - protein, 15 aa, Artificial Sequence
  VH-CDR3
VKDYYGTRPSIGMDY

SEQ ID NO: 11 - protein, 112 aa, Artificial Sequence
  VL of antibody 103G4
DIVMTQAAPSVPVTPGESVSISCRSSRSLLHSNGNTYLYWFLQRPGQSPHLLIYRMSNLASGVPDRFSGSGSGTAFTLRISRVEAEDVGVYYCMQHLEYPFTFGAGTKLELK

SEQ ID NO: 12 - protein, 11 aa, Artificial Sequence
  VL-CDR1
RSLLHSNGNTY

SEQ ID NO: 13 - protein, 9 aa, Artificial Sequence
  VL-CDR3
FQGSGYPLT

SEQ ID NO: 14 - protein, 9 aa, Artificial Sequence
  VL-CDR3
MQHLEYPFT

SEQ ID NO: 15 - protein, 124 aa, Artificial Sequence
  VH of antibody 118H1
EVQLVESGGGLVQPKGSLKLSCAASGFSFNPYAMNWVRQAPGKGLEWIARIRSKSNNYAAYYADSVKDRFTISRDDSENILYLQMKNLITEDTAMYYCVRDYYGTRPSIGMDYWGRGTSVTVSS

SEQ ID NO: 16 - protein, 10 aa, Artificial Sequence
  VH-CDR2
IRSKSNNYAA

SEQ ID NO: 17 - protein, 15 aa, Artificial Sequence
  VH-CDR3
VRDYYGTRPSIGMDY

SEQ ID NO: 18 - protein, 112 aa, Artificial Sequence
  VL of antibody 118H1
DIAMTQAAPSVFVTPGESVSISCRSSQSLLHSNGNTYLYWFLQRPGRSPQLLIYRMSNLASGVPDRFSGSGSGTGFTLRISRVEAEDVGVYYCMQHLEYPFTFGAGTKLELK

SEQ ID NO: 19 - protein, 11 aa, Artificial Sequence
  VL-CDR1
QSLLHSNGNTY

SEQ ID NO: 20 - protein, 3 aa, Artificial Sequence
  VL-CDR2
LVS

SEQ ID NO: 21 - protein, 112 aa, Artificial Sequence
  VH of antibody 170G6
EVQLQQSGPVLVKPGASVKMSCKASGYTFTDNYMNWVKQSHGKSLEWIGVINPYNGVTRYNQKFRGKATLTADKSSSTAFMDLNSLTSEDSAVYYCSNSLSWGPGTTLTVSS

SEQ ID NO: 22 - protein, 8 aa, Artificial Sequence
  VH-CDR1
GYTFTDNY

SEQ ID NO: 23 - protein, 8 aa, Artificial Sequence
  VH-CDR2
INPYNGVT

SEQ ID NO: 24 - protein, 5 aa, Artificial Sequence
  VH-CDR3
SNSLS

SEQ ID NO: 25 - protein, 112 aa, Artificial Sequence
  VL of antibody 170G6
DVVMTQSPLTLSVTIGQTASISCKSSQSLLDTDGKTYLNWLLQRPGQSPKRLIYLVSKLDSGVPDRFTGSGSGTDFTLKISRVEAEDLGVYYCWQGIHYPRTFGGGTKLEIK

SEQ ID NO: 26 - protein, 11 aa, Artificial Sequence
  VL-CDR1
QSLLDTDGKTY

SEQ ID NO: 27 - protein, 9 aa, Artificial Sequence
  VL-CDR3
WQGIHYPRT

SEQ ID NO: 28 - protein, 112 aa, Artificial Sequence
  VH of antibody 172E7
EVQLQQSGPVLVKPGASVKMSCKASGYTFTDNYMNWMKQSHGKTLEWIGVINPYNGVTRYNQKFRDKATLTVDKSSSTAYMDLNSLTSEDSAVYYCSNSLSWGPGTSLTVSS

SEQ ID NO: 29 - protein, 112 aa, Artificial Sequence
  VL of antibody 172E7
DVVMTQTPLTLSVTIGQAASISCKSSQSLVDSDGKTYLNWLLQRPGQSPKRLIYLVSKLDSGVPDRFTGSGSGTDFTLKISRVEAEDLGVYYCWQGIHYPRTFGGGTKLEII

SEQ ID NO: 30 - protein, 11 aa, Artificial Sequence
  VL-CDR1
QSLVDSDGKTY

SEQ ID NO: 31 - protein, 122 aa, Artificial Sequence
  VH of antibody 84B4
EVQLQQSGPVLVKPGASVKMSCKASGFTITDYYFNWVKQSHGKSLEWIGIINPYNGVARYKQKFKGKATLTVDKSSSTVYLEFSGLTSEDSAVYYCVRFVYGTTYDYAMDYWGQGTSVTVSS

SEQ ID NO: 32 - protein, 8 aa, Artificial Sequence
  VH-CDR1
GFTITDYY

SEQ ID NO: 33 - protein, 8 aa, Artificial Sequence
  VH-CDR2
INPYNGVA

SEQ ID NO: 34 - protein, 15 aa, Artificial Sequence
  VH-CDR3
VRFVYGTTYDYAMDY

SEQ ID NO: 35 - protein, 112 aa, Artificial Sequence
  VL of antibody 84B4
DVLMTQTPLSLPVSLGDQASISCRSSQSIVHSNGNTYLEWYVQKPGQSPKLLIYKVSNRFSGVPDRFSGSGSGTDFTLKISRVEAEDLGVYYCFQGSHVPPTFGGGTKLEIK

SEQ ID NO: 36 - protein, 11 aa, Artificial Sequence
  VL-CDR1
QSIVHSNGNTY

SEQ ID NO: 37 - protein, 3 aa, Artificial Sequence
  VL-CDR2
KVS

SEQ ID NO: 38 - protein, 9 aa, Artificial Sequence
  VL-CDR3
FQGSHVPPT

SEQ ID NO: 39 - protein, 123 aa, Artificial Sequence
  VH of antibody 2P15
EVQLVESGGGLVQPKGSLKLSCAASGFSFNAYAMNWVRQAPGKGLEWVARMRSKSNYYATYYADSVKDRFTISRDDSESMLYLQMNNLKTEDTAMYYCVRQTYGSKDYAMDYWGQGTSVTVSS

SEQ ID NO: 40 - protein, 8 aa, Artificial Sequence
  VH-CDR1
GFSFNAYA

SEQ ID NO: 41 - protein, 10 aa, Artificial Sequence
  VH-CDR2
MRSKSNYYAT

SEQ ID NO: 42 - protein, 14 aa, Artificial Sequence
  VH-CDR3
VRQTYGSKDYAMDY

SEQ ID NO: 43 - protein, 112 aa, Artificial Sequence
  VL of antibody 2P15
DVVMTQTPLSLPVSLGDQASISCRSSQSLVHSNGNTYFHWYLQKPGQSPKLLIYKVSNRFSGVPDRFSGSGSGTDFTLKISRVEAEDLGVYFCSQSTHVPFTFGSGTKLEIK

SEQ ID NO: 44 - protein, 11 aa, Artificial Sequence
  VL-CDR1
QSLVHSNGNTY

SEQ ID NO: 45 - protein, 9 aa, Artificial Sequence
  VL-CDR3
SQSTHVPFT

SEQ ID NO: 46 - protein, 116 aa, Artificial Sequence
  VH of antibody 3C11
EFQLQQSGPELVKPGASVKISCKTSGYSFTDYNINWVKQSNGKSLEWIGLINPNHGTTKSNQKFKGKATLTVDQSSSTAYMQLSSLTSEDSAVYYCARHDYDGAYWGQGTLVTVSA

SEQ ID NO: 47 - protein, 8 aa, Artificial Sequence
  VH-CDR1
GYSFTDYN

SEQ ID NO: 48 - protein, 8 aa, Artificial Sequence
  VH-CDR2
INPNHGTT

SEQ ID NO: 49 - protein, 9 aa, Artificial Sequence
  VH-CDR3
ARHDYDGAY

SEQ ID NO: 50 - protein, 112 aa, Artificial Sequence
  VL of antibody 3C11
DVVMTQTPLSLPVSLGDQASISCRSSQSLIHSNGNTYLHWYLQKPGQSPKLLIYKISNRFSGVPDRFSGSGSGTDFTLKISRVEAEDLGVYFCSQSTRAPWTFGGGTKLEIK

SEQ ID NO: 51 - protein, 11 aa, Artificial Sequence
  VL-CDR1
QSLIHSNGNTY

SEQ ID NO: 52 - protein, 3 aa, Artificial Sequence
  VL-CDR2
KIS

SEQ ID NO: 53 - protein, 9 aa, Artificial Sequence
  VL-CDR3
SQSTRAPWT

SEQ ID NO: 54 - protein, 123 aa, Artificial Sequence
  VH of antibody 3O20
EVQLVESGGGLVQPKGSLKLSCATSGFSFNAYAMNWVRQAPGKGLEWVGRIRSKSNNYATYYADSVKGRFTISRDDSKTILYLQMNTLKTEDTAIYYCVRGGYHGNSAYFDVWGTGTSVTVSS

SEQ ID NO: 55 - protein, 14 aa, Artificial Sequence
  VH-CDR3
VRGGYHGNSAYFDV

SEQ ID NO: 56 - protein, 112 aa, Artificial Sequence
  VL of antibody 3O20
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCLQHLEYPFTFGSGTKLEIK

SEQ ID NO: 57 - protein, 123 aa, Artificial Sequence
  VH of antibody 4M13
EVQLVESGGGLVQPKGSLKLSCAASGFSFNTYAMNWVRQAPGKGLEWVARMRSKSNNYATYYADSVKDRFTISRDDSESMLYLQMNNLKTEDTAMYYCVRGKDTSGSYAMDYWGQGTSVSVSS

SEQ ID NO: 58 - protein, 8 aa, Artificial Sequence
  VH-CDR1
GFSFNTYA

SEQ ID NO: 59 - protein, 10 aa, Artificial Sequence
  VH-CDR2
MRSKSNNYAT

SEQ ID NO: 60 - protein, 14 aa, Artificial Sequence
  VH-CDR3
VRGKDTSGSYAMDY

SEQ ID NO: 61 - protein, 112 aa, Artificial Sequence
  VL of antibody 4M13
DIVMTQAAPSVPVTPGESVSISCRSSKSLLHSNGNTYLYWFLQRPGQSPHLLIYRMSNLASGVPDRFSGSGSGTAFTLRISKVETEDVGVYYCMQHLEYPFTFGSGTKLEIK

SEQ ID NO: 62 - protein, 123 aa, Artificial Sequence
  VH of antibody 2L15
EVQLVESGGGLVQPKGSLKLSCAASGFSFNTYAMNWVRQAPGKGLEWVARIRSKSNNYATYYADSVKDRFTISRADSESMLYLQMNNLKTEDTAMYYCVRGKDISVSYAMDYWGQGTSVTVSS

SEQ ID NO: 63 - protein, 14 aa, Artificial Sequence
  VH-CDR3
VRGKDISVSYAMDY

SEQ ID NO: 64 - protein, 112 aa, Artificial Sequence
  VL of antibody 2L15
DIVMTQAAPSVPVTPGESVSISCRSSKSLQHSNGNTYLYWFLQRPGQSPQLLIYRMSNLASGVPDRFSGSGSGTAFTLRISRVETEDVGVYYCMQHLEYPFTFGSGTKLEIK

SEQ ID NO: 65 - protein, 11 aa, Artificial Sequence
  VL-CDR1
KSLQHSNGNTY

SEQ ID NO: 66 - protein, 121 aa, Artificial Sequence
  VH of antibody 4D24
EVQLVESGGGLVQPKGSLKLSCAASGFSFNPYAMNWVRQSPGKGLEWVARIRSKSNNYATYYADSVKDRFTISRDDSESMLYLQMNNLKTEDTAMYYCVRQGWVKRYFDVWGTGTTVTVSS

SEQ ID NO: 67 - protein, 12 aa, Artificial Sequence
  VH-CDR3
VRQGWVKRYFDV

SEQ ID NO: 68 - protein, 106 aa, Artificial Sequence
  VL of antibody 4D24
GNVLTQSPAIMSASPGEKVTMTCSASSSGTYMHWYQQKSTTSPKLWIYDTSKLASGVPGRFSGSGSGNSYSLTISSMEAEDIATYYCFQGSGYPLTFGSGTKLEIK

SEQ ID NO: 69 - protein, 5 aa, Artificial Sequence
  VL-CDR1
SSGTY

SEQ ID NO: 70 - protein, 3 aa, Artificial Sequence
  VL-CDR2
DTS

SEQ ID NO: 71 - protein, 115 aa, Artificial Sequence
  VH of antibody 1G17
QVQLQQSGAELVRPGASVTLSCKASGYTFADYEMHWVKQTPVLGLEWIGAIDPETGRTAYNQKFKFKATLTADRSSSTAYMELRSLTSEDSAVYYCTRRAYWGGWGQGTTLTVSS

SEQ ID NO: 72 - protein, 8 aa, Artificial Sequence
  HCDR1 of antibody 1G17
GYTFADYE

SEQ ID NO: 73 - protein, 8 aa, Artificial Sequence
  HCDR2 of antibody 1G17
IDPETGRT

SEQ ID NO: 74 - protein, 8 aa, Artificial Sequence
  HCDR3 of antibody 1G17
TRRAYWGG

SEQ ID NO: 75 - protein, 112 aa, Artificial Sequence
  VL of antibody 1G17
DVVMTQTPLSLPVSLGDQASISCRSSQSLVHSSGNTYLHWYLQKPGQSPKLLIYKVSNRFSGVPDRFSGSGSGTDFTLKISRVEAEDLGVYFCSQSTHVPYTFGGGTKLEIK

SEQ ID NO: 76 - protein, 11 aa, Artificial Sequence
  LCDR1 of antibody 1G17
QSLVHSSGNTY

SEQ ID NO: 77 - protein, 9 aa, Artificial Sequence
  LCDR3 of antibody 1G17
SQSTHVPYT

SEQ ID NO: 78 - protein, 123 aa, Artificial Sequence
  VH of antibody 3F11
EVQLVESGGGLVQPKGSLKLSCAASGFSFNTYAMNWVRQAPGKGLEWVARIRSKSNYYATYYADSVKDRFTISRDDSESMLYLQMNNLKTEDTAMYYCVRGRELGDYYAMDYWGQGTSVTVSS

SEQ ID NO: 79 - protein, 10 aa, Artificial Sequence
  HCDR2 of antibody 3F11
IRSKSNYYAT

SEQ ID NO: 80 - protein, 14 aa, Artificial Sequence
  HCDR3 of antibody 3F11
VRGRELGDYYAMDY

SEQ ID NO: 81 - protein, 112 aa, Artificial Sequence
  VL of antibody 3F11
DIVMTQAAPSVPVTPGESVSISCRSSKSLLHSNGNTYLYWFLQRPGQSPQLLIYRMSNLASGVPNRFSGSGSGTAFTLRISRVEAEDVGVYYCMQHLEYPFTFGSGTKLEIK

SEQ ID NO: 82 - protein, 124 aa, Artificial Sequence
  VH of antibody 3F6
EVQFVESGGGLVQPKGSLKLSCAASGFSFNTYAMNWVRQAPGKGLEWVARIRTKSNNYATHYADSVKDRFIVSRDDSENILYLQMNNLKTEDTGMYYCVRGGNGIYGRNAMDNWGQGTSVTVSS

SEQ ID NO: 83 - protein, 10 aa, Artificial Sequence
  HCDR2 of antibody 3F6
IRTKSNNYAT

SEQ ID NO: 84 - protein, 15 aa, Artificial Sequence
  HCDR3 of antibody 3F6
VRGGNGIYGRNAMDN

SEQ ID NO: 85 - protein, 112 aa, Artificial Sequence
  VL of antibody 3F6
DIVMTQAAPSVPVSPGESVSISCRSSKSLLHSSGNTYLYWFLQRPGQSPQLLIYRMSNLASGVPDRFSGSGSGTAFTLRISRVEAEDVGVYYCMQHLEYPFTFGGGTKLEIK

SEQ ID NO: 86 - protein, 11 aa, Artificial Sequence
  LCDR1 of antibody 3F6
KSLLHSSGNTY

SEQ ID NO: 87 - protein, 124 aa, Artificial Sequence
  VH of antibody 4G19
EVQFVESGGGLVQPKGSLKLSCAASGFSFNTYAMNWVRQAPGKGLEWVARIRTKSNNYATHYADSVTDRFIVSRDDSESMTYLQMNNLKTEDTGMYYCVRGGNGIYGRNTMDNWGQGTSVTVSS

SEQ ID NO: 88 - protein, 15 aa, Artificial Sequence
  HCDR3 of antibody 4G19
VRGGNGIYGRNTMDN

SEQ ID NO: 89 - protein, 112 aa, Artificial Sequence
  VL of antibody 4G19
DIVMTQTAPSVPVSPGESVSISCRSSKSLLHSSGNTYLYWFLQRPGQSPQLLIYRMSNLASGVPDRFSGSGSGTAFTLRISRVEAEDVGVYYCMQHLEYPFTFGGGTKLEIQ

SEQ ID NO: 90 - protein, 123 aa, Artificial Sequence
  VH of antibody 3F11hz1
EVQLVESGGGLVQPGGSLKLSCAASGFSFNTYAMHWVRQASGKGLEWVGRIRSKSNYYATYYAASVKGRFTISRDDSKNTAYLQMNSLKTEDTAVYYCTRGRELGDYYAMDYWGQGTLVTVSS

SEQ ID NO: 91 - protein, 112 aa, Artificial Sequence
  VL of antibody 3F11hz1
DIVMTQSPLSLPVTPGEPASISCRSSKSLLHSNGNTYLDWYLQKPGQSPQLLIYRMSNRASGVPDRFSGSGSGTDFTLKISRVEAEDVGVYYCMQHLEYPFTFGQGTKLEIK

SEQ ID NO: 92 - protein, 123 aa, Artificial Sequence
  VH of antibody 3F11hz2, 3F11hz5 and 3F11hz6
EVQLVESGGGLVQPGGSLKLSCAASGFSFNTYAMNWVRQASGKGLEWVGRIRSKSNYYATYYAASVKGRFTISRDDSKNTAYLQMNSLKTEDTAVYYCVRGRELGDYYAMDYWGQGTLVTVSS

SEQ ID NO: 93 - protein, 112 aa, Artificial Sequence
  VL of antibody 3F11hz2
DIVMTQSPLSLPVTPGEPASISCRSSKSLLHSNGNTYLDWFLQKPGQSPQLLIYRMSNRASGVPDRFSGSGSGTDFTLKISRVEAEDVGVYYCMQHLEYPFTFGQGTKLEIK

SEQ ID NO: 94 - protein, 123 aa, Artificial Sequence
  VH of antibody 3F11hz3
EVQLVESGGGLVQPGGSLKLSCAASGFTFSTYAMNWVRQASGKGLEWVGRIRSKSNYYATYYADSVKDRFTISRDDSKNTAYLQMNSLKTEDTAVYYCVRGRELGDYYAMDYWGQGTLVTVSS

SEQ ID NO: 95 - protein, 112 aa, Artificial Sequence
  VL of antibody 3F11hz3
DIVMTQSPLSLPVTPGEPASISCRSSKSLLHSNGNTYLDWFLQKPGQSPQLLIYRMSNLASGVPDRFSGSGSGTDFTLKISRVEAEDVGVYYCMQHLEYPFTFGQGTKLEIK

SEQ ID NO: 96 - protein, 123 aa, Artificial Sequence
  VH of antibody 3F11hz4
EVQLVESGGGLVQPGGSLKLSCAASGFSFNTYAMNWVRQASGKGLEWVGRIRSKSNYYATYYAASVKGRFTISRDDSKSTLYLQMNSLKTEDTAVYYCVRGRELGDYYAMDYWGQGTLVTVSS

SEQ ID NO: 97 - protein, 112 aa, Artificial Sequence
  VL of antibody 3F11hz4
DIVMTQSPLSLPVTPGEPASISCRSSKSLLHSNGNTYLYWFLQKPGQSPQLLIYRMSNLASGVPDRFSGSGSGTDFTLKISRVEAEDVGVYYCMQHLEYPFTFGQGTKLEIK

SEQ ID NO: 98 - protein, 112 aa, Artificial Sequence
  VL of antibody 3F11hz5
DIVMTQSPLSLPVTPGEPASISCRSSKSLLHSNGNTYLEWYLQKPGQSPQLLIYRMSNRASGVPDRFSGSGSGTDFTLKISRVEAEDVGVYYCMQHLEYPFTFGQGTKLEIK

SEQ ID NO: 99 - protein, 112 aa, Artificial Sequence
  VL of antibody 3F11hz6
DIVMTQSPLSLPVTPGEPASISCRSSKSLLHSSGNTYLDWFLQKPGQSPQLLIYRMSNRASGVPDRFSGSGSGTDFTLKISRVEAEDVGVYYCMQHLEYPFTFGQGTKLEIK

SEQ ID NO: 100 - protein, 123 aa, Artificial Sequence
  VH of antibody 3O20hz1
EVQLVESGGGLVQPGGSLKLSCAASGFSFNAYAMHWVRQASGKGLEWVGRIRSKSNNYATAYAASVKGRFTISRDDSKNTAYLQMNSLKTEDTAVYYCVRGGYHGNSAYFDVWGQGTLVTVSS

SEQ ID NO: 101 - protein, 112 aa, Artificial Sequence
  VL of antibody 3O20hz1
DIVMTQSPLSLPVTPGEPASISCRSSKSLLHSNGNTYLDWYLQKPGQSPQLLIYRMSNRASGVPDRFSGSGSGTDFTLKISRVEAEDVGVYYCLQHLEYPFTFGQGTKLEIK

SEQ ID NO: 102 - protein, 123 aa, Artificial Sequence
  VH of antibody 3O20hz2
EVQLVESGGGLVQPGGSLKLSCATSGFSFNAYAMNWVRQASGKGLEWVGRIRSKSNNYATYYAASVKGRFTISRDDSKTTLYLQMNSLKTEDTAVYYCVRGGYHGNSAYFDVWGQGTLVTVSS

SEQ ID NO: 103 - protein, 112 aa, Artificial Sequence
  VL of antibody 3O20hz2
DIVMTQSPLSLPVTPGEPASISCRSSKSLLHSNGNTYLYWFLQKPGQSPQLLIYRMSNLASGVPDRFSGSGSGTDFTLKISRVEAEDVGVYYCLQHLEYPFTFGQGTKLEIK

SEQ ID NO: 104 - protein, 123 aa, Artificial Sequence
  VH of antibody 3O20hz3
EVQLVESGGGLVQPGGSLKLSCATSGFSFNAYAMHWVRQASGKGLEWVGRIRSKSNNYATAYAASVKGRFTISRDDSKTTLYLQMNSLKTEDTAVYYCVRGGYHGNSAYFDVWGQGTLVTVSS

SEQ ID NO: 105 - protein, 112 aa, Artificial Sequence
  VL of antibody 3O20hz3
DIVMTQSPLSLPVTPGEPASISCRSSKSLLHSQGNTYLDWFLQKPGQSPQLLIYRMSNRASGVPDRFSGSGSGTDFTLKISRVEAEDVGVYYCLQHLEYPFTFGQGTKLEIK

SEQ ID NO: 106 - protein, 115 aa, Artificial Sequence
  VH of antibody 1G17hz1
QVQLVQSGAEVKKPGASVKVSCKASGYTFADYYMHWVRQAPGQGLEWMGAIDPETGRTAYAQKFQGRVTMTRDTSTSTVYMELSSLRSEDTAVYYCARRAYWGGWGQGTLVTVSS

SEQ ID NO: 107 - protein, 112 aa, Artificial Sequence
  VL of antibody 1G17hz1
DVVMTQSPLSLPVTLGQPASISCRSSQSLVHSSGNTYLHWFQQRPGQSPRRLIYKVSNRFSGVPDRFSGSGSGTDFTLKISRVEAEDVGVYYCSQSTHVPYTFGQGTKLEIK

SEQ ID NO: 108 - protein, 115 aa, Artificial Sequence
  VH of antibody 1G17hz2
QVQLVQSGAEVKKPGASVKVSCKASGYTFADYEMHWVRQAPGQGLEWIGIIDPETGRTSYAQKFQGRATLTADRSTSTAYMELSSLRSEDTAVYYCTRRAYWGGWGQGTLVTVSS

SEQ ID NO: 109 - protein, 112 aa, Artificial Sequence
  VL of antibody 1G17hz2
DVVMTQSPLSLPVTLGQPASISCRSSQSLVHSSGNTYLNWYQQRPGQSPRLLIYKVSNRDSGVPDRFSGSGSGTDFTLKISRVEAEDVGVYFCSQSTHVPYTFGQGTKLEIK

SEQ ID NO: 110 - protein, 115 aa, Artificial Sequence
  VH of antibody 1G17hz3
QVQLVQSGAEVKKPGASVKVSCKASGYTFADYEMHWVRQAPGQGLEWMGAIDPETGRTAYAQKFQGRVTMTADRSTSTAYMELSSLRSEDTAVYYCTRRAYWGGWGQGTLVTVSS

SEQ ID NO: 111 - protein, 112 aa, Artificial Sequence
  VL of antibody 1G17hz3
DVVMTQSPLSLPVTLGQPASISCRSSQSLVHSSGNTYLHWYQQRPGQSPRLLIYKVSNRFSGVPDRFSGSGSGTDFTLKISRVEAEDVGVYFCSQSTHVPYTFGQGTKLEIK

(19) United States
(12) Patent Application Publication
(10) Pub. No.: US 2024/0352134 A1
(43) Pub. Date: Oct. 24, 2024

(54) Anti-CCR8 Antibodies and Uses Thereof

(71) Applicant: NANJING IMMUNOPHAGE BIOTECH CO., LTD., Nanjing (CN)

(72) Inventors: Guohuang Fan, Nanjing (CN); Jianfei Wang, Nanjing (CN); Juan DU, Nanjing (CN); Na Wang, Nanjing (CN)

(21) Appl. No.: 18/573,978

(22) PCT Filed: Jun. 24, 2022
(86) PCT No.: PCT/CN2022/101000

§ 371 Date: Dec. 22, 2023

Related U.S. Application Data
(63) Continuation of application No. PCT/CN2022/092803, filed on May 13, 2022.
Application No. PCT/CN2022/092803 is a continuation of application No. PCT/CN2021/102324, filed on Jun. 25, 2021.

(51) Int. Cl. C07K 16/28; A61K 39/00; A61P 35/00

(52) U.S. Cl. CPC C07K 16/2866; A61P 35/00; A61K 2039/505; C07K 2317/24; C07K 2317/33; C07K 2317/565; C07K 2317/567; C07K 2317/76; C07K 2317/92

(57) ABSTRACT

The present invention relates to anti-CCR8 antibodies and to methods of using anti-CCR8 antibodies. The anti-CCR8 antibodies described herein are useful for the diagnosis and treatment of diseases mediated by CCR8 and/or CCL1, such as various cancers and neuropathic pain associated with abnormal CCL1/CCR8 axis.

### TECHNICAL FIELD

The present invention relates to novel anti-CCR8 antibodies, or antigen-binding fragment thereof, a nucleic acid encoding the antibody or the antigen-binding fragment thereof, a vector and a host cell including the nucleic acid, a method for producing the antibody or the antigen-binding fragment thereof, a pharmaceutical composition containing the antibody or the antigen-binding fragment thereof as an active ingredient, and the use of the antibody in treating the diseases mediated by CCR8.

### BACKGROUND OF THE INVENTION

Chemokines are a family of low molecular weight chemotactic cytokines involved in cell recruitment and activation in inflammation. Chemokines regulate a broad spectrum of cellular functions and exert their actions by binding to chemokine receptors which are G protein-coupled receptors, causing chemotaxis and activation of various subpopulations of cells in the immune system. Chemokines are divided into different classes, including CC, CXC, CX3C and XC, based on the positions of the N-terminal cysteine residues within the protein. The CC class of chemokines contains the CC motif in which the first two cysteines are not separated by any amino acids, whereas the CXC class of chemokines contains the CXC motif in which the first two cysteines are separated by a random amino acid. The activity of chemokines is mediated primarily through tight binding to their receptors on the surface of leukocytes.

In normal physiological conditions, the expression of chemokines and chemokine receptors are delicately and tightly regulated, dysregulated expression and activation of either chemokines and chemokine receptors often result in maladies such as autoimmune diseases or cancers.

The body has many intrinsic mechanisms intended to guard against cancer development. In this regard, the immune system is thought to play a key role in eradicating cells harboring genetic mutations. It follows, therefore, that cancer cells often persist by evolving ways to avoid recognition by the cells of the immune system. In particular, it has been shown that elevated levels of regulatory T lymphocytes (which may be referred to herein as “T_{reg} cells”), both within the peripheral circulation and within the tumor microenvironment, underlie the immune suppression seen in cancer patients. The presence of increased numbers of T_{reg} cells has also been identified as a barrier to the successful implementation of cancer immunotherapies.

CCR8 (C-C Motif Chemokine Receptor 8) is predominantly expressed on T_{reg} cells and Th₂ cells, but not on Th₁ cells. This subset of CD4⁺ Foxp3⁺ T_{reg} cells expressing CCR8 (CCR8+T_{reg} cells) has been demonstrated to be a major driver of immunosuppression and is critical for T_{reg} function and suppression. Moreover, CCR8 was a specific marker selectively upregulated by tumor-resident T_{reg} cells in several tumor types. Many reports show that the increase of CCR8+T_{reg} cells is beneficial to the tumor escape mechanism. In clinical, the increase of T_{reg} cells in tumor microenvironment of breast cancer, gastric cancer, ovarian cancer, pancreatic cancer, liver cancer, colon cancer and many other cancer types is associated with poor prognosis. In terms of mechanism, T_{reg} cells not only inhibit a wide range of anti-tumor immune responses but also promote the regeneration of tumor microenvironment blood vessels.

Cancer cells and immune cells in the tumor microenvironment secret CCL1, the specific ligand of CCR8, recruiting CCR8⁺ Treg cells to the tumor microenvironment. CCR8 also plays a role in Treg proliferation and expansion in the tumor microenvironment. CCR8 inhibitors have been shown to reduce tumor-infiltrated T_{reg} cells, thereby preventing tumor growth. Thus, CCR8 is considered a potential therapeutic target for cancer.

CCR8 is expressed in spinal cord neurons, which are also the main source of spinal CCL1. CCL1 is a well-characterized chemokine from the CC subfamily. It attracts immune cells by interacting with the cell surface chemokine receptor CCR8. CCL1 and CCR8 neuronal signaling also plays an important role in neuropathic pain induced by diabetes, spinal cord injury etc. Therefore, the CCL1/CCR8 axis might be a promising novel target for drug development to treat diabetic neuropathy.

CCL1-CCR8 interactions were also reported to play a critical role in IgG4-related diseases (IgG4-RD), such as IgG4-related sclerosing cholangitis (ISC).

Another CCR8 ligand recently identified was CCL18 which is a chemokine from the β-chemokine sub-family. The CCL18-CCR8 axis in labial salivary glands (LSGs) and lacrimal glands of IgG4-RD patients was specifically up-regulated compared with primary Sjögren's syndrome and control subjects. This axis might be a potentially novel therapeutic target in IgG4-RD, based on its important etiopathogenic roles, such as chemotaxis of various cells, induction of fibrosis, and enhancement of IgG4 production.

In light of the role that CCR8 plays in the pathogenesis of various diseases, it is desirable to prepare antibodies that inhibit CCR8 activity, which may be useful in the treatment of diseases mediated by CCR8, such as cancer, neuropathic pain, IgG4-related diseases (IgG4-RD), including but not limited to autoimmune pancreatitis, eosinophilic angiocentric fibrosis, fibrosing mediastinitis, hypertrophic pachymeningitis, idiopathic hypocomplementemic tubulointerstitialnephritis with extensive tubulointerstitial deposits, inflammatory aortic aneurysm, inflammatory pseudotumor, Küttner's tumor (chronic sclerosing sialadenitis), mediastinal fibrosis, Mikulicz's syndrome, multifocal fibrosclerosis, periaortitis and periarteritis, retroperitoneal fibrosis (Ormond's disease), Riedel's thyroiditis, sclerosing mesenteritis, sclerosing pancreatitis, and sclerosing cholangitis, etc.

Summing up, there is a need in the art for more effective therapeutics comprising anti-CCR8 antibodies that effectively inhibit the CCR8 signaling while causing minimal adverse side effects in humans.

### SUMMARY OF THE INVENTION

The purpose of the invention is to provide anti-CCR8 antibodies or antigen-binding fragments thereof that specifically bind CCR8, which activate the immunity by inhibiting immunosuppression mediated by T_{reg} cells or the like specifically expressing CCR8. In addition, the anti-CCR8 antibodies or antigen-binding fragments thereof disclosed herein can reduce neuropathic pain by regulating the CCL1/CCR8 axis. Such antibodies or antigen-binding fragments may be used to target cells expressing CCR8 for therapeutic and diagnostic purposes.

The inventors of the instant invention also found that the humanized antibodies disclosed herein not only bind specifically to CCR8, but also show a strong CCL1-CCR8 signaling block and improved species cross-reactivity. Therefore, the main advantages of the present invention include:
(a) the antibody according to the present invention has an excellent bioactivity and specificity, it has a good binding affinity for cell surface CCR8, and may be used as an CCR8-targeting antibody;
(b) the fully human antibody according to the present invention not only has an activity comparable to that of immune antibodies, but also has a lower immunogenicity;
(c) the antibody according to the present invention not only has a significant effect in inhibiting CCL1-induced chemotaxis, but also applicable to other abnormal CCL1/CCR8 axis associated diseases.

In the first aspect, it provides an antibody or an antigen-binding fragment thereof comprising at least one heavy chain variable region comprising a HCDR1, a HCDR2 and a HCDR3 selected from the group consisting of
a) SEQ ID NOs: 2, 3, and 4; or
b) SEQ ID NOs: 2, 3, and 10; or
c) SEQ ID NOs: 2, 16, and 17; or
d) SEQ ID NOs: 22, 23, and 24; or
e) SEQ ID NOs: 32, 33, and 34; or
f) SEQ ID NOs: 40, 41, and 42; or
g) SEQ ID NOs: 47, 48, and 49; or
h) SEQ ID NOs: 40, 3, and 55; or
i) SEQ ID NOs: 58, 59, and 60; or
j) SEQ ID NOs: 58, 3, and 63; or
k) SEQ ID NOs: 2, 3, and 67; or
l) SEQ ID NOs: 72, 73, and 74; or
m) SEQ ID NOs: 58, 79, and 80; or
n) SEQ ID NOs: 58, 83, and 84; or
o) SEQ ID NOs: 58, 83, and 88; or
   and comprising at least one light chain variable region comprising a LCDR1, a LCDR2 and a LCDR3 selected from the group consisting of:
a) SEQ ID NOs: 6, 7, and 8; or
b) SEQ ID NOs: 12, 7, and 14; or
c) SEQ ID NOs: 19, 7, and 14; or
d) SEQ ID NOs: 26, 20, and 27; or
e) SEQ ID NOs: 30, 20, and 27; or
f) SEQ ID NOs: 36, 37, and 38; or
g) SEQ ID NOs: 44, 37, and 45; or
h) SEQ ID NOs: 51, 52, and 53; or
i) SEQ ID NOs: 6, 7, and 8; or
j) SEQ ID NOs: 6, 7, and 14; or
k) SEQ ID NOs: 65, 7, and 14; or
l) SEQ ID NOs: 69, 70, and 13; or
m) SEQ ID NOs: 76, 37, and 77; or
n) SEQ ID NOs: 86, 7, and 14.
   wherein the antibody or an antigen-binding fragment thereof specifically binds CCR8; and any of the above amino acid sequences further includes a derivative sequence formed by optionally addition, deletion, modification, and/or substitution of 1-5 (or 1, 2, 3) amino acids, and capable of retaining CCR8 binding ability.

In preferred embodiments, the said antibody or an antigen-binding fragment thereof comprising a HCDR1, a HCDR2, a HCDR3, a LCDR1, a LCDR2 and a LCDR3 selected from the group consisting of:
a) SEQ ID NOs: 2, 3, 4, 6, 7, and 8; or
b) SEQ ID NOs: 2, 3, 10, 12, 7, and 14; or
c) SEQ ID NOs: 2, 16, 17, 19, 7, and 14; or
d) SEQ ID NOs: 22, 23, 24, 26, 20, and 27; or
e) SEQ ID NOs: 22, 23, 24, 30, 20, and 27; or
f) SEQ ID NOs: 32, 33, 34, 36, 37, and 38; or
g) SEQ ID NOs: 40, 41, 42, 44, 37, and 45; or
h) SEQ ID NOs: 47, 48, 49, 51, 52, and 53; or
i) SEQ ID NOs: 40, 3, 55, 6, 7, and 8; or
j) SEQ ID NOs: 58, 59, 60, 6, 7, and 14; or
k) SEQ ID NOs: 58, 3, 63, 65, 7, and 14; or
l) SEQ ID NOs: 2, 3, 67, 69, 70, and 13; or
m) SEQ ID NOs: 72, 73, 74, 76, 37, and 77; or
n) SEQ ID NOs: 58, 79, 80, 6, 7, and 14; or
o) SEQ ID NOs: 58, 83, 84, 86, 7, and 14; or
p) SEQ ID NOs: 58, 83, 88, 86, 7, and 14.

In preferred embodiments, the heavy chain further includes a heavy chain constant region and/or the light chain further includes a light chain constant region.

In preferred embodiments, the number of the added, deleted, modified and/or substituted amino acids in 3 HCDRs and 3 LCDRs of the antibody is 1-5 (such as 1-3, preferably 1-2, more preferably 1).

In preferred embodiments, the heavy chain variable region of the antibody further comprises a human or humanized framework region, and/or the light chain variable region of the antibody further comprises a human or humanized framework region.

In preferred embodiments, the said antibody or an antigen-binding fragment thereof comprise a heavy chain variable region and a light chain variable region selected from the group consisting of
a) a heavy chain variable region having the polypeptide sequence of SEQ ID NO: 1, and a light chain variable region having the polypeptide sequence of SEQ ID NO: 5; or
b) a heavy chain variable region having the polypeptide sequence of SEQ ID NO: 9, and a light chain variable region having the polypeptide sequence of SEQ ID NO: 11; or
c) a heavy chain variable region having the polypeptide sequence of SEQ ID NO: 15, and a light chain variable region having the polypeptide sequence of SEQ ID NO: 18; or
d) a heavy chain variable region having the polypeptide sequence of SEQ ID NO: 21, and a light chain variable region having the polypeptide sequence of SEQ ID NO: 25; or
e) a heavy chain variable region having the polypeptide sequence of SEQ ID NO: 28, and a light chain variable region having the polypeptide sequence of SEQ ID NO: 29; or
f) a heavy chain variable region having the polypeptide sequence of SEQ ID NO: 31, and a light chain variable region having the polypeptide sequence of SEQ ID NO: 35; or
g) a heavy chain variable region having the polypeptide sequence of SEQ ID NO: 39, and a light chain variable region having the polypeptide sequence of SEQ ID NO: 43; or
h) a heavy chain variable region having the polypeptide sequence of SEQ ID NO: 46, and a light chain variable region having the polypeptide sequence of SEQ ID NO: 50; or
i) a heavy chain variable region having the polypeptide sequence of SEQ ID NO: 54, and a light chain variable region having the polypeptide sequence of SEQ ID NO: 56; or
j) a heavy chain variable region having the polypeptide sequence of SEQ ID NO: 57, and a light chain variable region having the polypeptide sequence of SEQ ID NO: 61; or
k) a heavy chain variable region having the polypeptide sequence of SEQ ID NO: 62, and a light chain variable region having the polypeptide sequence of SEQ ID NO: 64; or
l) a heavy chain variable region having the polypeptide sequence of SEQ ID NO: 66, and a light chain variable region having the polypeptide sequence of SEQ ID NO: 68; or
m) a heavy chain variable region having the polypeptide sequence of SEQ ID NO: 71, and a light chain variable region having the polypeptide sequence of SEQ ID NO: 75; or
n) a heavy chain variable region having the polypeptide sequence of SEQ ID NO: 78, and a light chain variable region having the polypeptide sequence of SEQ ID NO: 81; or
o) a heavy chain variable region having the polypeptide sequence of SEQ ID NO: 82, and a light chain variable region having the polypeptide sequence of SEQ ID NO: 85; or
p) a heavy chain variable region having the polypeptide sequence of SEQ ID NO: 87, and a light chain variable region having the polypeptide sequence of SEQ ID NO: 89; or
q) a heavy chain variable region having the polypeptide sequence of SEQ ID NO: 90, and a light chain variable region having the polypeptide sequence of SEQ ID NO: 91; or
r) a heavy chain variable region having the polypeptide sequence of SEQ ID NO: 92, and a light chain variable region having the polypeptide sequence of SEQ ID NO: 93; or
s) a heavy chain variable region having the polypeptide sequence of SEQ ID NO: 94, and a light chain variable region having the polypeptide sequence of SEQ ID NO: 95; or
t) a heavy chain variable region having the polypeptide sequence of SEQ ID NO: 96, and a light chain variable region having the polypeptide sequence of SEQ ID NO: 97; or
u) a heavy chain variable region having the polypeptide sequence of SEQ ID NO: 92, and a light chain variable region having the polypeptide sequence of SEQ ID NO: 98; or
v) a heavy chain variable region having the polypeptide sequence of SEQ ID NO: 92, and a light chain variable region having the polypeptide sequence of SEQ ID NO: 99; or
w) a heavy chain variable region having the polypeptide sequence of SEQ ID NO: 100, and a light chain variable region having the polypeptide sequence of SEQ ID NO: 101; or
x) a heavy chain variable region having the polypeptide sequence of SEQ ID NO: 102, and a light chain variable region having the polypeptide sequence of SEQ ID NO: 103; or
y) a heavy chain variable region having the polypeptide sequence of SEQ ID NO: 104, and a light chain variable region having the polypeptide sequence of SEQ ID NO: 105; or
z) a heavy chain variable region having the polypeptide sequence of SEQ ID NO: 106, and a light chain variable region having the polypeptide sequence of SEQ ID NO: 107; or
aa) a heavy chain variable region having the polypeptide sequence of SEQ ID NO: 108, and a light chain variable region having the polypeptide sequence of SEQ ID NO: 109; or
bb) a heavy chain variable region having the polypeptide sequence of SEQ ID NO: 110, and a light chain variable region having the polypeptide sequence of SEQ ID NO: 111.

In preferred embodiments, the antibody is a monoclonal antibody.

In preferred embodiments, the antibody is a double-chain antibody or a single-chain antibody.

In preferred embodiments, the antibody is a full-length antibody protein or an antigen-binding fragment.

In preferred embodiments, the antibody is a recombinant antibody.

In preferred embodiments, the antibody or an antigen-binding fragment thereof is chimeric.

In preferred embodiments, the antibody is a bispecific antibody or a multispecific antibody.

In another preferred embodiment, the CCR8 specific antibody is selected from the group consisting of (i) a single chain antibody, a single-chain variable fragment (scFv), a univalent antibody lacking a hinge region or a minibody; (ii) a Fab, Fab′ or F(ab′)₂ fragment; (iii) a whole antibody; and (iv) an antibody that comprises a human IgG Fc domain.

In preferred embodiments, the antibody or an antigen-binding fragment thereof is human or humanized.

In preferred embodiments, the antibody is human anti-CCR8 antibody.

In preferred embodiments, the antibody or an antigen-binding fragment thereof comprises a heavy chain variable region having a polypeptide sequence at least (≥) 85%, at least 90%, at least 95%, at least 96%, at least 97%, at least 98%, or at least 99% identical to SEQ ID NO: 1, 9, 15, 21, 28, 31, 39, 46, 54, 57, 62, 66, 71, 78, 82, 87, 90, 92, 94, 96, 100, 102, 104, 106, 108, or 110, or a light chain variable region having a polypeptide sequence at least 85%, at least 90%, at least 95%, at least 96%, at least 97%, at least 98%, or at least 99% identical to SEQ ID NO: 5, 11, 18, 25, 29, 35, 43, 50, 56, 61, 64, 68, 75, 81, 85, 89, 91, 93, 95, 97, 98, 99, 101, 103, 105, 107, 109, or 111.

In particular preferred embodiments, the V_{H} chain and the V_{L} chain of the CCR8 specific antibodies have at least 80%, preferably at least 90%, more preferably at least 95%, and even more preferably at least 99% sequence identity, respectively, with the amino acid sequences of the respective V_{H} chain and V_{L} chain selected from the group consisting of: SEQ ID NOs: 1 and 5; or SEQ ID NOs: 9 and 11; or SEQ ID NOs: 15 and 18; or SEQ ID NOs: 21 and 25; or SEQ ID NOs: 28 and 29; or SEQ ID NOs: 31 and 35; or SEQ ID NOs: 39 and 43; or SEQ ID NOs: 46 and 50; or SEQ ID NOs: 54 and 56; or SEQ ID NOs: 57 and 61; or SEQ ID NOs: 62 and 64; or SEQ ID NOs: 66 and 68; or SEQ ID NOs: 71 and 75; or SEQ ID NOs: 78 and 81; or SEQ ID NOs: 82 and 85; or SEQ ID NOs: 87 and 89; or SEQ ID NOs: 90 and 91; or SEQ ID NOs: 92 and 93; or SEQ ID NOs: 94 and 95; or SEQ ID NOs: 96 and 97; or SEQ ID NOs: 92 and 98; or SEQ ID NOs: 92 and 99; or SEQ ID NOs: 100 and 101; or SEQ ID NOs: 102 and 103; or SEQ ID NOs: 104 and 105; or SEQ ID NOs: 106 and 107; or SEQ ID NOs: 108 and 109; or SEQ ID NOs: 110 and 111.

In some embodiments, disclosed herein is an antibody or antigen-binding fragment binds to human CCR8 at an epitope comprising one or more amino acid residues selected from the group consisting of Y94 to K107 of human CCR8.

In some embodiments, disclosed herein is an antibody or antigen-binding fragment binds to human CCR8 at an epitope comprising one or more amino acid residues selected from the group consisting of Y94, L95, L96, D97, Q98, V100, T103, V104, M105, and K107 of human CCR8.

In some embodiments, disclosed herein is an antibody or antigen-binding fragment binds to human CCR8 at an epitope comprising one amino acid residue selected from the group consisting of Y94, L95, L96, Q98, V100, T103, V104, M105, and K107 of human CCR8.

In other particular preferred embodiments, the CCR8 specific antibody is an IgA, an IgD, an IgE, an IgG, or an IgM antibody.

In another preferred embodiment, the CCR8 specific antibody is an IgG selected from the group consisting of IgG1, IgG2, IgG3, IgG4, and synthetic IgG.

In preferred embodiments, the antibody is in the form of a drug conjugate.

In the second aspect, it provides a recombinant protein (or polypeptide) which comprises:
(i) the antibody or an antigen-binding fragment thereof according to the first aspect of the invention; and
(ii) optional tag sequences to assist expression and/or purification.

In preferred embodiments, the tag sequence comprises a 6His tag.

In preferred embodiments, the recombinant protein (or polypeptide) includes a fusion protein.

In preferred embodiments, the recombinant protein is a monomer, a dimer, or a multimer.

In the third aspect, it provides isolated nucleic acids encoding the monoclonal antibodies or antigen-binding fragments in the first aspect, or the recombinant protein in the second aspect of the invention.

In the third aspect, it provides isolated nucleic acids encoding the antibodies or antigen-binding fragments in the first aspect, or the recombinant protein in the second aspect of the invention.

In the fourth aspect, it provides a vector which comprises the isolated nucleic acids encoding the antibodies or antigen-binding fragments in the first aspect, or the recombinant protein in the second aspect of the invention.

In preferred embodiments, the vector comprises bacterial plasmids, phages, yeast plasmid, plant cell virus, mammalian cell viruses such as adenovirus, lentivirus, retrovirus, or other vectors.

In the fifth aspect, it provides an antibody conjugate which comprises:
(i) an antibody moiety selected from the group consisting of an antibody or an antigen-binding fragment thereof in the first aspect of the invention, or a recombinant protein in the second aspect of the invention, or a combination thereof; and
(ii) a coupling moiety coupled to the antibody moiety, wherein the coupling moiety is selected from the group consisting of a detectable label, a drug, a toxin, a cytokine, a radionuclide, an enzyme, or a combination thereof.

In preferred example, the antibody moiety and the coupling moiety are coupled by a chemical bond or a linker.

In the sixth aspect, it provides a pharmaceutical composition which comprises (i) the antibody or an antigen-binding fragment thereof in the first aspect, the recombinant protein in the second aspect, the isolated nucleic acids (especially DNA or RNA) in the third aspect, the vector in the fourth aspect, the antibody conjugate in the fifth aspect, or combinations thereof, and (ii) a pharmaceutically acceptable carrier.

In preferred embodiments, the said antibody has an effect of removing tumor-infiltrating Tᵣg cells.

In the seventh aspect, it provides a method for treating a disease mediated by CCR8 and/or CCL1, which comprises administering an effective amount of the antibody or an antigen-binding fragment thereof in the first aspect, the recombinant protein in the second aspect, the isolated nucleic acids (especially DNA or RNA) in the third aspect, the vector in the fourth aspect, the antibody conjugate in the fifth aspect, or the pharmaceutical composition in the seventh aspect or combinations thereof, to a subject in need; or the use of the antibody or an antigen-binding fragment thereof in the first aspect, the recombinant protein in the second aspect, the isolated nucleic acids (especially DNA or RNA) in the third aspect, the vector in the fourth aspect, the antibody conjugate in the fifth aspect, or the pharmaceutical composition in the seventh aspect or combinations thereof in the manufacture of a medicament for treating a disease mediated by CCR8 and/or CCL1; or the antibody or an antigen-binding fragment thereof in the first aspect, the recombinant protein in the second aspect, the isolated nucleic acids (especially DNA or RNA) in the third aspect, the vector in the fourth aspect, the antibody conjugate in the fifth aspect, or the pharmaceutical composition in the seventh aspect or combinations thereof for use in treating a disease mediated by CCR8 and/or CCL1.

In preferred embodiments, the disease mediated by CCR8 and/or CCL1 is cancer.

In a particular preferred embodiment, the cancer is breast cancer, gastric cancer, ovarian cancer, pancreatic cancer, liver cancer, colon cancer, or pancreatic cancer.

In a preferred embodiment, the disease is associated with an abnormal CCL1/CCR8 axis.

In a particular preferred embodiment, the disease associated with the abnormal CCL1/CCR8 axis is neuropathic pain.

In a particular preferred embodiment, the neuropathic pain induced by diabetes or spinal cord injury.

In a particular preferred embodiment, the disease is IgG4-related diseases.

In a particular preferred embodiment, the IgG4-related diseases is sclerosing cholangitis, autoimmune pancreatitis, eosinophilic angiocentric fibrosis, fibrosing mediastinitis, hypertrophic pachymeningitis, idiopathic hypocomplementemic tubulointerstitialnephritis with extensive tubulointerstitial deposits, inflammatory aortic aneurysm, inflammatory pseudotumor, Küttner's tumor (chronic sclerosing sialadenitis), mediastinal fibrosis, Mikulicz's syndrome, multifocal fibrosclerosis, periaortitis and periarteritis, retroperitoneal fibrosis (Ormond's disease), Riedel's thyroiditis, sclerosing mesenteritis, or sclerosing pancreatitis.

In the eighth aspect, it provides a method of determining a level of CCR8 in a subject which comprise (a) obtaining a sample from the subject; (b) contacting the sample with an isolated monoclonal antibody or an antigen-binding fragment thereof of the invention, and (c) determining a level of CCR8 in the subject.

Preferably, the sample is a tissue sample or a blood sample and the tissue sample can be a cancer tissue sample.

In the ninth aspect, it provides a use of an active ingredient for (a) preparation of a diagnostic reagent or kit; and/or (b) preparation of a medicament for the prevention and/or treatment of a disease associated with CCR8, wherein the active ingredient is selected from the group consisting of the antibody or an antigen-binding fragment thereof in the first aspect, the recombinant protein in the second aspect, the isolated nucleic acids (especially DNA or RNA) in the third aspect, the vector in the fourth aspect, the antibody conjugate in the fifth aspect, and combinations thereof.

In preferred embodiments, the diagnostic reagent is a test strip or a test plate.

In preferred embodiments, the disease associated with CCR8 comprises cancer.

In a particular preferred embodiment, the cancer is breast cancer, gastric cancer, ovarian cancer, pancreatic cancer, liver cancer, colon cancer, or pancreatic cancer.

In preferred embodiments, the diagnostic reagent or kit is used for:
(i) detecting CCR8 protein in a sample; and/or
(ii) detecting endogenous CCR8 protein in spinal cord neurons; and/or
(iii) detecting regulatory T lymphocytes expressing CCR8 protein.

In preferred embodiments, the antibody is in the form of an antibody-drug conjugate (ADC).

In the tenth aspect, it provides a method for in vitro detection (including diagnostic or non-diagnostic detection) of a CCR8 protein in a sample which comprises:
(i) contacting the sample in vitro with an antibody in the first aspect of the invention; and
(ii) detecting whether an antigen-antibody complex is formed, where the formation of the complex indicates the presence of CCR8 protein in the sample.

In the eleventh aspect, it provides a kit which comprises:
(i) a first container containing the antibody of the present invention as a first antibody; and
(ii) a second container containing a secondary antibody against the first antibody of the present invention.

In a thirteenth aspect, it provides a method for preparing a recombinant polypeptide, which comprises:
(i) culturing an engineered host cell in the fifth aspect of the invention under conditions suitable for expression; and
(ii) isolating a recombinant polypeptide from the culture, wherein the recombinant polypeptide is an antibody or an antigen-binding fragment thereof in the first aspect or the recombinant protein in the second aspect.

### BRIEF DESCRIPTION OF THE DRAWINGS

FIGS. 1A and 1B depicts that the antibodies have binding ability to CCR8 overexpressed 293F cells (293F-CCR8) in FACS binding analysis for clones 103G4, 1181H1, 101E4, 84B4, 170G6, 172E7, 2P15, 3C11, 3O20, 4M13, 2L15, 4D24, 1G17, 3F11, 3F6, and 4G19; FIGS. 1C and 1D depicts that the antibodies did not bind with parental 293F cells in FACS binding analysis for some clones.

FIGS. 2A, 2B, 2C, 2D, and 2E show the results of anti-tumor efficacy of anti-CCR8 antibodies, i.e., antibody clone 84B4, 101E4, 170G6, 3C11, 2P15, respectively, in MDA-MB-231 xenograft.

FIG. 3 show the results of 3F11hz4 and 3F11 binding affinity to mouse CCR8 CHOK1.

FIGS. 4A, 4B, 4C, 4D and 4E show the antibody 3F11hz4 has the binding ability to human CCR8 overexpressed 293T cells (human CCR8-293T), rat CCR8 overexpressed 293T cells (rat CCR8-293T), dog CCR8 overexpressed 293T cells (dog CCR8-293T), mouse CCR8 overexpressed CHOK1 cells (mouse CCR8-CHOK1) and cynomolgus CCR8 overexpressed 293T cells (cyno CCR8-293T), respectively, in FACS binding analysis using 293T cell line and CHOK1 cell line.

FIGS. 5A and 5B show 3F11hz4 binding affinity to mutant CCR8 293T.

FIGS. 6A and 6B show the results of 3F11hz4 binding affinity to hCCR4 293T and hCX3CR1 293T.

FIG. 7 shows anti-tumor efficacy of 3F11hz4 antibody against HCC827 lung cancer subcutaneous tumor (CD34 humanized model).

FIG. 8 shows the anti-tumor efficacy of 3F11hz4 Antibody against H22 liver cancer subcutaneous tumor (Syngeneic model).

FIG. 9 shows the anti-tumor efficacy of 3F11hz4 Antibody against LLC lung cancer subcutaneous tumor (Syngeneic model).

FIG. 10 shows migration test of Tregs cells.

### DETAILED DESCRIPTION OF THE INVENTION

Through extensive and intensive research, the inventors unexpectedly obtained a set of fully human CCR8 antibodies with a completely new amino acid sequence. The CCR8 antibodies of the present invention have excellent high affinity with CCR8 protein, thus is useful for treating CCR8-related diseases such as allo-immune diseases, auto-immune diseases, allergy, inflammatory diseases, tumors, neuropathic pain, or IgG4-related diseases. The present invention has been completed on this basis.

### Terms

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a”, “an”, and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises”, “comprising”, “includes”, “including”, “has”, “having”, “contains” or “containing”, or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of”, or variations such as “consist of” or “consisting of”, as used throughout the specification and claims, indicate the inclusion of any recited integer or group of integers, but that no additional integer or group of integers can be added to the specified method, structure, or composition.

As used herein, the term “consists essentially of”, or variations such as “consist essentially of” or “consisting essentially of”, as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

The words “right”, “left”, “lower”, and “upper” designate directions in the drawings to which reference is made.

It should also be understood that the terms “about”, “approximately”, “generally”, “substantially”, and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity”, in the context of two or more nucleic acids or polypeptide sequences (e.g., anti-CCR8 antibodies and polynucleotides that encode them, CCR8 polypeptides and CCR8 polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSEIM62 scoring matrix (see Henikoff & Henikoff, Proc. Natl. Acad. Sci. ETSA 89: 10915 (1989)).

In addition to calculating percent sequence identity, the BLAST algorithm also performs a statistical analysis of the similarity between two sequences (see, e.g., Karlin & Altschul, Proc. NatT. Acad. Sci. ETSA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide”, “polypeptide”, and “protein” are used interchangeably and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types, “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “antigen-binding fragment” as used herein refers to a polypeptide fragment that contains at least one CDR of an immunoglobulin heavy and/or light chain that binds to the antigen of interest, which antigen in particularly preferred embodiments described herein is the C-C Motif Chemokine Receptor 8 (CCR8). In this regard, an antigen-binding fragment of the herein described antibodies may comprise one, two, three, four, five or all six CDRs of a V_{H} and/or V_{L} sequence set forth herein from antibodies that bind CCR8. An antigen-binding fragment of the herein described CCR8-specific antibodies is capable of binding to CCR8. In other embodiments, binding of an antigen-binding fragment prevents or inhibits binding of CCR8 ligand(s) to the CCR8 receptor, interrupting the biological response that would otherwise result from ligand binding to the receptor. In certain embodiments, the antigen-binding fragment binds specifically to and/or inhibits or modulates the biological activity of CCR8.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes.

The term “epitope” includes any determinant, preferably a polypeptide determinant, that is capable of specific binding to an immunoglobulin or T-cell receptor. An epitope is a region of an antigen that is bound by an antibody. In certain embodiments, epitope determinants include chemically active surface groupings of molecules such as amino acids, sugar side chains, phosphoryl or sulfonyl, and may in certain embodiments have specific three-dimensional structural characteristics, and/or specific charge characteristics. In certain embodiments, an antibody is said to specifically bind an antigen when it preferentially recognizes its target antigen in a complex mixture of proteins and/or macromolecules. An antibody may according to certain embodiments be said to bind an antigen specifically when the equilibrium dissociation constant for antibody-antigen binding is less than or equal to 10⁻⁶ M, or less than or equal to 10⁻⁷ M, or less than or equal to 10⁻⁸ M. In some embodiments, the equilibrium dissociation constant may be less than or equal to 10⁻⁹ M or less than or equal to 10⁻¹⁰ M.

The term “vector” is used to refer to any molecule (e.g., nucleic acid, plasmid, or virus) used to transfer coding information to a host cell. The term “expression vector” refers to a vector that is suitable for the transformation of a host cell and contains nucleic acid sequences that direct and/or control the expression of inserted heterologous nucleic acid sequences. Expression includes, but is not limited to, processes such as transcription, translation, and RNA splicing, if introns are present.

### C-C Motif Chemokine Receptor 8 (CCR8)

CCR8, also previously called Cy6, CKR-L1 or TERI, is a G protein-coupled 7-transmembrane CC chemokine receptor protein expressed in the thymus, the spleen, etc. A gene encoding this protein resides on human chromosome 3p21. Human CCR8 consists of 355 amino acids. CCL1 is known as an endogenous ligand for CCR8. Human CCR8 cDNA is constituted by the nucleotide sequence represented by GenBank ACC No. M_005201.3, and mouse CCR8 cDNA is constituted by the nucleotide sequence represented by GenBank ACC No. NM_007720.2.

The CCR8 of the present invention includes those derived from mice, rats, hamsters, guinea pigs, dogs, pigs, and primate mammals including monkeys and humans. Human CCR8 is preferred.

### Antibodies

The invention generally relates to isolated anti-CCR8 antibodies, nucleic acids and expression vectors encoding the antibodies, recombinant cells containing the vectors, and compositions comprising the antibodies. Methods of making the antibodies, and methods of using the antibodies to treat diseases including cancer are also provided. The antibodies of the invention possess one or more desirable functional properties, including but not limited to high-affinity binding to CCR8, high specificity to CCR8, and the ability to inhibit tumor growth in subjects in need thereof and in animal models when administered alone or in combination with other anti-cancer therapies.

In a general aspect, the invention relates to isolated monoclonal antibody or an antigen-binding fragment thereof that specifically binds CCR8.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the invention can be of any of the five major classes or corresponding sub-classes. Preferably, the antibodies of the invention are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the invention can contain a kappa or lambda light chain constant domain. According to particular embodiments, the antibodies of the invention include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term an “isolated antibody” refers to an antibody which is substantially free of other antibodies having different antigenic specificities (e.g., an isolated antibody that specifically binds to CCR8 is substantially free of antibodies that do not bind to CCR8). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that may be present in minor amounts. The monoclonal antibodies of the invention can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)2, a bispecific dsFv (dsFv-dsFv1), a disulfide stabilized diabody (ds diabody), a single-chain antibody molecule (scFv), a single domain antibody (sdab) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to embodiments, the antigen-binding fragment comprises a light chain variable region, a light chain constant region, and an Fd segment of the heavy chain. According to other embodiments, the antigen-binding fragment comprises Fab and F(ab′).

As used herein, the term “single-chain antibody” refers to a conventional single chain antibody in the field, which comprises a heavy chain variable region and a light chain variable region connected by a short peptide of about 15 to about 20 amino acids. As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, the first epitope is located on CCR8 and the second epitope is located on PD-1, PD-L1, LAG-3, TIM-3, CTLA-4, EGFR, HER-2, CD19, CD20, CD33, CD47, CD73, apelin, DLL3, claudin18.2, TIP-1, CD3 and/or other tumor associated immune suppressors or surface antigens.

By the term “specifically binds” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding”, can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

In certain embodiments, antibodies and antigen-binding fragments thereof as described herein include a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1”, “CDR2”, and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures-regardless of the precise CDR amino acid sequence. Further, certain FR residues are known to participate in non-covalent interdomain contacts which stabilize the interaction of the antibody heavy and light chains.

The structures and locations of immunoglobulin variable regions may be determined by reference to Kabat, E. A. et al, Sequences of Proteins of Immunological Interest, 4th Edition, US Department of Health and Human Services, 1987, and updates thereof, now available on the Internet (immuno.bme.nwu.edu), Chothia, AbM and IMGT (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997) ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996). For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under IMGT, the CDR amino acid residues in the VH are numbered approximately 26-35 (HCDR1), 51-57 (HCDR2) and 93-102 (HCDR3), and the CDR amino acid residues in the VL are numbered approximately 27-32 (LCDR1), 50-52 (LCDR2), and 89-97 (LCDR3) (numbering according to Kabat). Under IMGT, the CDR regions of an antibody can be determined using the program IMGT/DomainGap Align. Unless specified otherwise, the positions of the CDRs and framework regions disclosed herein are determined under the IMGT numbering scheme.

An “antibody heavy chain” as used herein refers to the larger of the two types of polypeptide chains present in all antibody molecules in their naturally occurring conformations. The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

An “antibody light chain” as used herein refers to the smaller of the two types of polypeptide chains present in all antibody molecules in their naturally occurring conformations. K and X light chains refer to the two major antibody light chain isotypes.

By the term “synthetic antibody” as used herein is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The antibody of the present invention may be fused at its N terminus or C terminus with an additional protein (Clinical Cancer Research, 2004, 10, 1274-1281). The protein to be fused can be appropriately selected by those skilled in the art.

In preferred embodiments, the isolated monoclonal antibody or an antigen-binding fragment thereof comprises a HCDR1, a HCDR2, a HCDR3, a LCDR1, a LCDR2, and a LCDR3. The polypeptide sequences of HCDR1, HCDR2, HCDR3, LCDR1, LCDR2, and LCDR3 have selected from the group consisting of
1. SEQ ID NOs: 2, 3, 4, 6, 7, and 8; or
2. SEQ ID NOs: 2, 3, 10, 12, 7, and 14; or
3. SEQ ID NOs: 2, 16, 17, 19, 7, and 14; or
4. SEQ ID NOs: 22, 23, 24, 26, 20, and 27; or
5. SEQ ID NOs: 22, 23, 24, 30, 20, and 27; or
6. SEQ ID NOs: 32, 33, 34, 36, 37, and 38; or
7. SEQ ID NOs: 40, 41, 42, 44, 37, and 45; or
8. SEQ ID NOs: 47, 48, 49, 51, 52, and 53; or
9. SEQ ID NOs: 40, 3, 55, 6, 7, and 8; or
10. SEQ ID NOs: 58, 59, 60, 6, 7, and 14; or
11. SEQ ID NOs: 58, 3, 63, 65, 7, and 14; or
12. SEQ ID NOs: 2, 3, 67, 69, 70, and 13; or
13. SEQ ID NOs: 72, 73, 74, 76, 37, and 77; or
14. SEQ ID NOs: 58, 79, 80, 6, 7, and 14; or
15. SEQ ID NOs: 58, 83, 84, 86, 7, and 14; or
16. SEQ ID NOs: 58, 83, 88, 86, 7, and 14,
   wherein the antibody or an antigen-binding fragment thereof specifically binds CCR8, preferably human CCR8, wherein the positions of the CDRs are determined under the IMGT numbering scheme.

According to another particular aspect, the invention relates to an isolated monoclonal antibody or an antigen-binding fragment thereof comprising a heavy chain variable region having a polypeptide sequence at least 85%, preferably 90%, more preferably 95% or more, such as 95%, 96%, 97%, 98%, or 99% identical to one of SEQ ID NO: 1, 9, 15, 21, 28, 31, 39, 46, 54, 57, 62, 66, 71, 78, 82, 87, 90, 92, 94, 96, 100, 102, 104, 106, 108, or 110, or a light chain variable region having a polypeptide sequence at least 85%, preferably 90%, more preferably 95% or more, such as 95%, 96%, 97%, 98%, or 99% identical to one of SEQ ID NO: 5, 11, 18, 25, 29, 35, 43, 50, 56, 61, 64, 68, 75, 81, 85, 89, 91, 93, 95, 97, 98, 99, 101, 103, 105, 107, 109, or 111.

In the present invention, the antibody of the present invention also includes a conservative variant thereof, which means that, compared to the amino acid sequence of the antibody of the present invention, there are up to 10, preferably up to 8 and more preferably up to 5, most preferably up to 3 amino acids are replaced by amino acids with similar or similar properties to form a polypeptide. These conservative variant polypeptides are preferably produced by amino acid substitution according to Table A.

TABLE A

| Original residue | Representative replacement | Preferred replacement |
|---|---|---|
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

The invention relates to an isolated nucleic acid encoding a monoclonal antibody or an antigen-binding fragment thereof of the invention. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding a monoclonal antibody or an antigen-binding fragment thereof of the invention can be altered without changing the amino acid sequences of the proteins.

### Polynucleotides, Vectors, Host Cell and Method of Preparation

The invention also provides polynucleotides encoding any one of the antibodies disclosed herein. In some embodiment, disclosed herein is an solated polynucleotide encoding an antibody or antibody fragment which binds to CCR8, wherein the antibody or antibody fragment comprises three light chain CDRs having the amino acid sequences set forth in SEQ ID NOs:58, 79 and 80; and/or three heavy chain CDRs having the amino acid sequences set forth in SEQ ID NOs: 6, 7 and 14. In some embodiment, disclosed herein is an solated polynucleotide encoding an antibody or antibody fragment which binds to CCR8, wherein the antibody or antibody fragment comprises three light chain CDRs having the amino acid sequences set forth in SEQ ID NOs:58, 79 and 80; and three heavy chain CDRs having the amino acid sequences set forth in SEQ ID NOs: 6, 7 and 14. In some embodiments, disclosed herein is an solated polynucleotide encoding an antibody or antibody fragment which binds to CCR8, wherein the antibody or antibody fragment comprises a heavy chain variable region and a light chain variable region having the polypeptide sequence of selected from SEQ ID NOs: 96 and 97; SEQ ID NOs: 78 and 81; SEQ ID NOs: 90 and 91; SEQ ID NOs: 92 and 93; SEQ ID NOs: 94 and 95; SEQ ID NOs: 92 and 98; or SEQ ID NOs: 92 and 99.

The invention also provides a vector comprising an isolated nucleic acid molecule encoding a monoclonal antibody or an antigen-binding fragment thereof of the invention. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for the production of an antibody or an antigen-binding fragment thereof in the cell. Conventional cloning techniques or artificial gene synthesis can be used to generate a recombinant expression vector according to embodiments of the invention. Such techniques are well known to those skilled in the art in view of the present disclosure.

The invention also provides a host cell comprising an isolated nucleic acid molecule encoding a monoclonal antibody or an antigen-binding fragment thereof of the invention. Any host cell known to those skilled in the art in view of the present disclosure can be used for recombinant expression of antibodies or antigen binding fragments thereof of the invention. In some embodiments, the host cells are E. coli TG1 or BL21 cells (for expression of, e.g., an scFv or Fab antibody), CHO-DG44 cells, 293F cells, CHO-K1 cells or HEK293 cells (for expression of, e.g., a full-length IgG antibody). According to embodiments, the recombinant expression vector is transformed into host cells by conventional methods such as chemical transfection, heat shock, or electroporation, where it is stably integrated into the host cell genome such that the recombinant nucleic acid is effectively expressed.

The invention also provides to a method of producing a monoclonal antibody or an antigen-binding fragment thereof of the invention, comprising culturing a cell comprising a nucleic acid encoding the monoclonal antibody or antigen binding fragment thereof under conditions to produce a monoclonal antibody or antigen binding fragment thereof of the invention, and recovering the antibody or an antigen-binding fragment thereof from the cell or cell culture (e.g., from the supernatant). Expressed antibodies or antigen-binding fragments thereof can be harvested from the cells and purified according to conventional techniques known in the art and as described herein.

As will be understood by those skilled in the art, polynucleotides may include genomic sequences, extra-genomic and plasmid-encoded sequences and smaller engineered gene segments that express, or may be adapted to express, proteins, polypeptides, peptides and the like. Such segments may be naturally isolated, or modified synthetically by the skilled person.

As will also be recognized by the skilled artisan, polynucleotides may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide according to the present disclosure, and a polynucleotide may, but need not, be linked to other molecules and/or support materials. Polynucleotides may comprise a native sequence or may comprise a sequence that encodes a variant or derivative of such a sequence.

Typically, polynucleotide variants will contain one or more substitutions, additions, deletions and/or insertions, preferably such that the binding affinity of the antibody encoded by the variant polynucleotide is not substantially diminished relative to an antibody encoded by a polynucleotide sequence specifically set forth herein.

The polynucleotides described herein, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10000, about 5000, about 3000, about 2000, about 1000, about 500, about 200, about 100, about 50 base pairs in length, and the like, (including all intermediate lengths) are contemplated to be useful.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

### Antibody-Dependent Cell-Mediated Cytotoxicity (ADCC)

Antibody-dependent cell-mediated cytotoxicity (ADCC) refer to a cell-mediated reaction in which non-specific cytotoxic cells (e.g., Natural Killer (NK) cells, neutrophils, and macrophages) recognize bound antibody on a target cell and subsequently cause lysis of the target cell. In preferred embodiments, such cells are human cells. While not wishing to be limited to any particular mechanism of action, these cytotoxic cells that mediate ADCC generally express Fe receptors (FcRs). The primary cells for mediating ADCC, NK cells, express FeγRIII, whereas monocytes express FcγRI, FeγRII, FeγRIII and/or FcγRIV. FcR expression on hematopoietic cells is summarized in Ravetch and Kinet, Annu. Rev. Immunol., 9:457-92 (1991). To assess ADCC activity of a molecule, an in vitro ADCC assay such as that described in U.S. Pat. No. 5,500,362 or 5,821,337 may be performed. Useful effector cells for such assays include peripheral blood mononuclear cells (PBMC) and NK cells. Alternatively, or additionally, ADCC activity of the molecules of interest may be assessed in vivo, e.g., in an animal model such as that disclosed in Clynes et al., PNAS (USA), 95:652-656 (1998).

“Effector cells” are leukocytes which express one or more FcRs and perform effector functions. Preferably, the cells express at least FeγRI, FCγRII, FeγRIII and/or FcγRIV and carry out ADCC effector function. Examples of human leukocytes which mediate ADCC include PBMCs, NK cells, monocytes, cytotoxic T cells and neutrophils; with PBMCs and NK cells being preferred. In preferred embodiments the effector cells are human cells.

The terms “Fe receptor” or “FcR” are used to describe a receptor that binds to the Fe region of an antibody. The preferred FcR is a native sequence human FcR. Moreover, a preferred FcR is one which binds an IgG antibody (a gamma receptor) and includes receptors of the FcγRI, FeγRII, FeγRIII, and FcγRIV subclasses, including allelic variants and alternatively spliced forms of these receptors. FeγRII receptors include FeγRIIA (an “activating receptor”) and FeγRIIB (an “inhibiting receptor”), which have similar amino acid sequences that differ primarily in the cytoplasmic domains thereof. Activating receptor FeγRIIA contains an immunoreceptor tyrosine-based activation motif (ITAM) in its cytoplasmic domain. Inhibiting receptor FeγRIIB contains an immunoreceptor tyrosine-based inhibition motif (ITIM) in its cytoplasmic domain. (See, Daëron, Annu. Rev. Immunol., 15:203-234 (1997)). FcRs are reviewed in Ravetech and Kinet, Annu. Rev. Immunol., 9:457-92 (1991); Capel et al., Immunomethods, 4:25-34 (1994); and de Haas et al., J. Lab. Clin. Med., 126:330-41 (1995). Other FcRs, including those to be identified in the future, are encompassed by the term “FCR” herein. The term also includes the neonatal receptor, FcRn, which is responsible for the transfer of maternal IgGs to the fetus (Guyer et al., Immunol., 117:587 (1976) and Kim et al., J. Immunol., 24:249 (1994)).

### Complement Dependent Cytotoxicity (CDC)

Complement dependent cytotoxicity (CDC) refers to the ability of a molecule to initiate complement activation and lyse a target in the presence of complement. The complement activation pathway is initiated by the binding of the first component of the complement system (C1q) to a molecule (e.g., an antibody) complexed with a cognate antigen. To assess complement activation, a CDC assay, e.g., as described in Gazzano-Santaro et al., J. Immunol. Methods, 202:163 (1996), may be performed.

### Antibody-Drug Conjugate (ADC)

The present invention also provides an antibody-drug conjugate (ADC) based on the antibody according to the present invention.

Typically, the antibody-drug conjugate comprises the antibody and an effector molecule, wherein the antibody is conjugated to the effector molecule, and chemical conjugation is preferred. Preferably, the effector molecule is a therapeutically active drug. In addition, the effector molecule may be one or more of a toxic protein, a chemotherapeutic drug, a small-molecule drug or a radionuclide.

The antibody according to the present invention and the effector molecule may be coupled by a coupling agent. Examples of the coupling agent may be any one or more of a non-selective coupling agent, a coupling agent utilizing a carboxyl group, a peptide chain, and a coupling agent utilizing a disulfide bond. The non-selective coupling agent refers to a compound that results in a linkage between an effector molecule and an antibody via a covalent bond, such as glutaraldehyde, etc. The coupling agent utilizing a carboxyl group may be any one or more of cis-aconitic anhydride coupling agents (such as cis-aconitic anhydride) and acyl hydrazone coupling agents (the coupling site is acyl hydrazone).

Certain residues on an antibody (such as Cys or Lys, etc.) are used to link a variety of functional groups, including imaging agents (such as chromophores and fluorophores), diagnostic agents (such as MRI contrast agents and radioisotopes), stabilizers (such as poly (ethylene glycol)) and therapeutic agents. An antibody can be conjugated to a functional agent to form a conjugate of the antibody-functional agent. A functional agent (e.g., a drug, a detection reagent, a stabilizer) is conjugated (covalently linked) to an antibody. A functional agent can be linked to an antibody either directly or indirectly via a linker.

Antibodies can be conjugated to drugs to form antibody-drug conjugates (ADCs). Typically, an ADC comprises a linker between a drug and an antibody. The linker can be a degradable or non-degradable linker. Typically, degradable linkers are easily degraded in an intracellular environment, for example, the linker is degraded at the target site, thereby releasing the drug from the antibody. Suitable degradable linkers include, for example, enzyme-degradable linkers, including peptidyl-containing linkers that can be degraded by protease (e.g., lysosomal protease or endosomal protease) in a cell, or sugar linkers, for example, glucuronide-containing linkers that can be degraded by glucuronidase. Peptidyl linkers may include, for example, dipeptides, such as valine-citrulline, phenylalanine-lysine or valine-alanine. Other suitable degradable linkers include, for example, pH sensitive linkers (e.g., linkers that are hydrolyzed at a pH of below 5.5, such as hydrazone linkers) and linkers that are degraded under reducing conditions (e.g. disulfide-bond linkers). A non-degradable linker typically releases a drug under conditions that the antibody is hydrolyzed by protease.

Prior to linkage to an antibody, a linker has a reactive group capable of reacting with certain amino acid residues, and the linkage is achieved by the reactive group. A thiol-specific reactive group is preferred, which includes, for example, a maleimide compound, a halogenated (e.g. iodo-, bromo- or chloro-substituted) amide; a halogenated (e.g. iodo-, bromo- or chloro-substituted) ester; a halogenated (e.g. iodo-, bromo- or chloro-substituted) methyl ketone, a benzyl halide (e.g. iodide, bromide or chloride); vinyl sulfone, pyridyl disulfide; a mercury derivative such as 3,6-di-(mercurymethyl)dioxane, wherein the counter ion is CH₃COO⁺, Cl⁺ or NO₃⁺; and polymethylene dimethyl sulfide thiosulfonate. The linker may include, for example, a maleimide linked to an antibody via thiosuccimide.

A drug may be any cytotoxic, cytostatic or immunosuppressive drug. In an embodiment, an antibody is linked to a drug via a linker, and the drug has a functional group that can form a bond with the linker. For example, a drug may have an amino group, a carboxyl group, a thiol group, a hydroxyl group, or a ketone group that can form a bond with a linker. When a drug is directly linked to a linker, the drug has a reactive group before being linked to an antibody.

Useful drugs include, for example, anti-tubulin drugs, DNA minor groove binding agents, DNA replication inhibitors, alkylating agents, antibiotics, folic acid antagonists, antimetabolites, chemotherapy sensitizers, topoisomerase inhibitors, vinca alkaloids, etc. Examples of particularly useful cytotoxic drugs include, for example, DNA minor groove binding agents, DNA alkylating agents, and tubulin inhibitors; typical cytotoxic drugs include, for example, auristatins, camptothecins, docamycin/duocarmycins, etoposides, maytansines and maytansinoids (e.g. DM1 and DM4), taxanes, benzodiazepines or benzodiazepine containing drugs (e.g. pyrrolo[1,4]benzodiazepines (PBDs), indolinobenzodiazepines and oxazolidinobenzodiazepines), and vinca alkaloids.

In the present invention, a drug-linker can be used to form an ADC in a simple step process. In other embodiments, a bifunctional linker compound can be used to form an ADC in a two-step or multi-step process. For example, a cysteine residue is reacted with the reactive moiety of a linker in a first step, and then the functional group on the linker is reacted with a drug in the subsequent step, so as to form an ADC.

In general, the functional group on a linker is selected so that it can specifically react with the suitable reactive group on a drug moiety. As a non-limiting example, an azide-based moiety can be used to specifically react with the reactive alkynyl group on a drug moiety. The drug is covalently bound to the linker by 1,3-dipolar cycloaddition between the azide and alkynyl group. Other useful functional groups include, for example, ketones and aldehydes (suitable for reacting with hydrazides and alkoxyamines), phosphines (suitable for reacting with azides); isocyanates and isothiocyanates (suitable for reacting with amines and alcohols); and activated esters, for example, N-hydroxysuccinimide esters (suitable for reacting with amines and alcohols). These and other linkage strategies, for example, those described in Bioconjugation Technology (2nd Edition (Elsevier)), are well known to those skilled in the art. Those skilled in the art could understand that when a complementary pair of reactive functional groups are selected for a selective reaction between a drug moiety and a linker, each member of the complementary pair can be used for the linker, and can also be used for the drug.

The present invention further provides a method for preparing an ADC, which may further comprise: under conditions sufficient to form an antibody-drug conjugate (ADC), binding an antibody to a drug-linker compound.

In certain embodiments, the method according to the present invention comprises: under conditions sufficient to form an antibody-linker conjugate, binding an antibody to a bifunctional linker compound. In these embodiments, the method according to the present invention further comprises: under conditions sufficient to covalently link the drug moiety to the antibody via a linker, binding the antibody-linker conjugate to the drug moiety.

In some embodiments, an antibody-drug conjugate (ADC) has a formula as follows:

Ab-(LU-D)p

wherein:
Ab is an antibody,
LU is a linker;
D is a drug;
   and the subscript p is a value selected from 1 to 8.

### Pharmaceutical Compositions and Other Uses

CCL1/CCR8 signaling is an important pathway in the pathogenesis of several diseases including cancer, inflammatory diseases and diabetic neuropathy etc. In particular, the antibodies described herein specifically bind to CCR8 with unexpectedly high affinity and in certain embodiments have the ability for blocking the CCR8 signaling, inhibiting CCL1-induced chemotaxis. CCR8⁺ T_{reg} cells are known to be beneficial to the tumor escape mechanism. In some aspects, provided herein are methods of decreasing the number or activity of tumor infiltrating T regulatory cells (TITR) in a tumor present in a subject by inhibiting immunosuppression mediated by CCR8⁺ T_{reg} cells or the like and to provide a pharmaceutical composition for cancer treatment via this mechanism. Such cancer includes, and without limitation, breast cancer, gastric cancer, ovarian cancer, pancreatic cancer, liver cancer, colon cancer, pancreatic cancer and many other cancer types are associated with poor prognosis. In some aspects, provided herein are methods of increasing the amount of T effector cells in a tumor in a subject by administering to the subject anti-CCR8 antibody. The cytotoxicity may be antibody-dependent cell-mediated cytotoxicity (ADCC) or complement dependent cytotoxicity (CDC). The agent may be an antibody, for example, peptide, small molecule, a protein drug conjugate, or an interfering nucleic acid. In addition, the present invention can also regulate the CCL1/CCR8 axis that might treat diseases such as diabetic neuropathy, spinal cord injury, and IgG4-related diseases such as sclerosing cholangitis (ISC). Amino acid sequences of illustrative antibodies, or antigen-binding fragments thereof, or complementarity determining regions (CDRs) thereof.

The invention also provides a pharmaceutical composition, comprising an isolated monoclonal antibody or an antigen-binding fragment thereof of the invention and a pharmaceutically acceptable carrier. The term “pharmaceutical composition” as used herein means a product comprising an active ingredient of the invention together with a pharmaceutically acceptable carrier, wherein the active ingredient is selected from the group consisting of the isolated monoclonal antibody or an antigen-binding fragment thereof in the first aspect, the recombinant protein in the second aspect, the isolated nucleic acids (especially DNA or RNA) in the third aspect, the vector in the fourth aspect, the antibody conjugate in the sixth aspect, the immune cell in the seventh aspect, or combinations thereof. The active ingredient of the invention and compositions comprising them are also useful in the manufacture of a medicament for therapeutic applications mentioned herein.

To “treat” a disease as the term is used herein refers to reduce the frequency or severity of at least one sign or symptom of a disease or disorder experienced by a subject.

The amount administered will depend on variables such as the type and extent of disease or indication to be treated, the overall health of the patient, the in vivo potency of the antibody, the pharmaceutical formulation, the serum half-life of the antibody, and the route of administration.

Administration frequency can vary, depending on factors such as route of administration, dosage amount, serum half-life of the antibody or fusion protein, and the disease being treated.

In some embodiments, antibodies of the invention are used for non-therapeutic purposes, such as diagnostic tests and assays. For example, the antibodies are useful for determining a level of CCR8 in a sample from a subject. A method by contacting the sample with a CCR8-specific antibody of the invention and detecting immunoreactivity between the antibody and CCR8 in the sample is provided.

### Detection Application and Kit

The antibody or an ADC thereof according to the present invention can be used in detection applications, for example, for use in the detection of a sample so as to provide diagnostic information.

In the present invention, the specimen (sample) used includes a cell, a tissue sample and a biopsy specimen. The term “biopsy” used in the present invention should include all kinds of biopsies known by those skilled in the art. Therefore, the biopsy specimen used in the present invention may include, for example, a resected sample of a tumor, and a tissue sample prepared by endoscopic methods or puncture or needle puncture biopsy of an organ.

The sample used in the present invention includes a fixed or preserved cell or tissue sample.

The present invention further provides a kit only comprising the antibody (or a fragment thereof) according to the present invention; in a preferred example of the present invention, the kit further comprises containers, instructions, buffers, etc. In the preferred examples, the antibody according to the present invention can be immobilized on a test panel.

In accordance with a further aspect of the invention, a CCR8 mediated disease is diagnosed in a subject by detecting the presence or quantity of CCR8 protein in a sample.

The present invention provides a kit for predicting or diagnosing the prognosis of cancers, the kit comprising the anti-CCR8 antibody. The kit of the present invention may further comprise tools and/or reagents known in the art which are used for ELISA. The kit of the present invention may further comprise, if necessary, tubes which are to be used to mix respective components, well plates, instruction manuals describing how to use, or the like.

The present invention is further described by reference to the following examples. It should be understood that the following examples are only used to describe the present invention, rather than limiting the scope of the present invention. The experimental methods in the following examples, the specific conditions of which are not indicated, are usually carried out according to conventional conditions, for example, the conditions described in Sambrook et al., Molecular Cloning: Laboratory Manual (New York: Cold Spring Harbor Laboratory Press, 1989), or the conditions recommended by the manufacturers. Unless otherwise specified, percentages and parts refer to percentages by weight and parts by weight. Cell lines are the conventional products that are commercially available or are purchased from ATCC, and all the plasmids are the products that are commercially available.

### Cell Lines:

The 293F cell line was obtained from Thermo Fisher (R79007) and was cultured in Expi293 TM Expression Medium.

### Example 1. Generation of Anti-CCR8 Monoclonal Antibodies

Anti-CCR8 monoclonal antibodies were developed by immunizing SJL mice with 293F cells overexpressing CCR8. Briefly, 293F Cells were transfected with lentiviral vectors encoding CCR8 by polybrene (8 μg/mL), selected in media containing puromycin (2 μg/mL), and tested for the expression of CCR8 by FACS. Individual clones with the greatest MFI to CCR8 were selected for subsequent studies.

Spleen and lymph nodes cells from these mice were fused with myeloma cells (SP20) by standard methods to generate hybridomas producing unique antibodies. Supernatants containing antibodies produced by pools of these cells were tested by cell-based ELISA for reactivity with CCR8 overexpressing cells. Cell-based ELISA was generally carried out as follows. Approximately 3×10⁴ 293F-CCR8 cells per well were seeded in 96-well plate, and after overnight culture, the cells were washed with 1×PBS-T, followed by addition of 100 μL of 4% paraformaldehyde solution to fix and cross-link the cells to the microplate. The cells were washed with 1×PBS-T twice. Then the cells were incubated with neat supernatants taken from hybridoma cultures including blank and positive control at 37° C. for 60 minutes. Subsequently, the cells were washed with 1×PBS-T four times. The cells were then incubated with goat anti-mouse IgG second antibody at 1:10000 dilution in 100 μL PBS at 37° C. for 60 minutes, followed by four times washing with 1×PBS-T. One-hundred μL TMB was added into the 96-well plate. After 10-12 minutes incubation, the plate was scanned with detection at 450 μm wavelength.

Supernatants of these positive clones were then confirmed by fluorescence-activated cell sorting (FACS). FACS analyses were generally carried out as follows. Approximately 5×10⁵ 293F-CCR8 cells per sample were prepared and blocked with Mouse BD Fc Block. The cells were distributed into 96-well round-bottom polystyrene plates and incubated with neat supernatants taken from hybridoma cultures on ice for 20-30 minutes. Next, the cells were washed with PBS/0.5% BSA and were centrifuged. The pelleted cell samples were then incubated with the second antibody of anti-mouse IgG labeled with FJTC at 1:300 dilution in 100 μL PBS/0.5% BSA on ice for 30 minutes, and then washed with PBS/0.5% BSA and spun down. The cell pellets were re-suspended in PBS/0.5% BSA, and the samples were analyzed on a CYTOFLEX (Beckman). Generally, the same supernatants were tested on non-transfected parental cells to confirm that the reactive antibody recognized CCR8 specifically.

Positive pools were identified and subcloned by limiting dilution. After three fusions, nine clones producing unique antibodies which recognized CCR8 overexpression cells specifically by FACS were obtained, they were: 101E4, 172E7, 103G4, 118H1, 170G6, 84B4, 2P15, 3C11, 3O20, 4M13, 2L15, 4D24, 1G1′7, 3F11, 3F6 and 4G19. The hybridomas were referred to the same designation as the antibodies produced where from (e.g., hybridoma 84B4 produces antibody 84B4). All the anti-CCR8 antibody clones have high affinity antibodies whose amino acid sequences of V_{H} and V_{L} were shown in Table 1.

TABLE 1
The amino acid sequence of the anti-CCR8 antibodies of clones C3, G3 and G6 (wherein
CDRs including HCDR1, HCDR2, HCDR3, LCDR1, LCDR2, LCDR3 are underlined and
are based on IMGT numbering scheme).

| | | |
|---|---|---|
| Name or | | SEQ ID No./ |
| region | Sequence | Position |

101E4

| | | |
|---|---|---|
| V_{H} | EVQLVESGGGLVQPKGSLKLSCAASGFSFNPYAMNWVRQGPGKGLE | SEQ ID No: 1 |
| | WVARIRSKSNNYATYYADSVKDRFTISRDDSENILYLQMNNLKTEDT | |
| | AMYYCVRDYYGGRSSIGMDYWGHGTSVTVSS | |
| HCDR1 | GFSFNPYA | SEQ ID No: 2 |
| HCDR2 | IRSKSNNYAT | SEQ ID No: 3 |
| HCDR3 | VRDYYGGRSSIGMDY | SEQ ID No: 4 |
| HFR1 | EVQLVESGGGLVQPKGSLKLSCAAS | Position 1-25 of |
| | | SEQ ID No: 1 |
| HFR2 | MNWVRQGPGKGLEWVAR | Position 34-50 of |
| | | SEQ ID No: 1 |
| HFR3 | YYADSVKDRFTISRDDSENILYLQMNNLKTEDTAMYYC | Position 61-98 of |
| | | SEQ ID No: 1 |
| HFR4 | WGHGTSVTVSS | Position 114-124 of |
| | | SEQ ID No: 1 |
| V_{L} | DIVMTQAAPSVPVTPGESVSISCRSSKSLLHSNGNTYLYWFLQRPGQS | SEQ ID No: 5 |
| | PQLLIYRMSNLASGVPDRFSGSGSGTAFTLRISRVEAEDVGVYYCLQH | |
| | LEYPFTFGGGTKLELK | |
| LCDR1 | KSLLHSNGNTY | SEQ ID No: 6 |
| LCDR2 | RMS | SEQ ID No: 7 |
| LCDR3 | LQHLEYPFT | SEQ ID No: 8 |
| LFR1 | DIVMTQAAPSVPVTPGESVSISCRSS | Position 1-26 of |
| | | SEQ ID No: 5 |
| LFR2 | LYWFLQRPGQSPQLLIY | Position 38-54 of |
| | | SEQ ID No: 5 |
| LFR3 | NLASGVPDRFSGSGSGTAFTLRISRVEAEDVGVYYC | Position 58-93 of |
| | | SEQ ID No: 5 |
| LFR4 | FGGGTKLELK | Position 103-112 of |
| | | SEQ ID No: 5 |

103G4

| | | |
|---|---|---|
| V_{H} | EVQLVESGGGLVRPKGSLKLSCAASGFSFNPYALNWVRQAPGKGLE | SEQ ID No: 9 |
| | WVARIRSKSNNYATYYADSVKDRFTISRDDSENILYLQMNNLKTEDT | |
| | AMYYCVKDYYGTRPSIGMDYWGQGTSVTVSS | |
| HCDR1 | GFSFNPYA | SEQ ID No: 2 |
| HCDR2 | IRSKSNNYAT | SEQ ID No: 3 |
| HCDR3 | VKDYYGTRPSIGMDY | SEQ ID No: 10 |
| HFR1 | EVQLVESGGGLVRPKGSLKLSCAAS | Position 1-25 of |
| | | SEQ ID No: 9 |
| HFR2 | LNWVRQAPGKGLEWVAR | Position 34-50 of |
| | | SEQ ID No: 9 |
| HFR3 | YYADSVKDRFTISRDDSENILYLQMNNLKTEDTAMYYC | Position 61-98 of |
| | | SEQ ID No: 9 |
| HFR4 | WGQGTSVTVSS | Position 114-124 of |
| | | SEQ ID No: 9 |
| V_{L} | DIVMTQAAPSVPVTPGESVSISCRSSRSLLHSNGNTYLYWFLQRPGQS | SEQ ID No: 11 |
| | PHLLIYRMSNLASGVPDRFSGSGSGTAFTLRISRVEAEDVGVYYCMQ | |
| | HLEYPFTFGAGTKLELK | |
| LCDR1 | RSLLHSNGNTY | SEQ ID No: 12 |
| LCDR2 | RMS | SEQ ID No: 7 |
| LCDR3 | MQHLEYPFT | SEQ ID No: 14 |
| LFR1 | DIVMTQAAPSVPVTPGESVSISCRSS | Position 1-26 of |
| | | SEQ ID No: 11 |
| LFR2 | LYWFLQRPGQSPHLLIY | Position 38-54 of |
| | | SEQ ID No: 11 |
| LFR3 | NLASGVPDRFSGSGSGTAFTLRISRVEAEDVGVYYC | Position 58-93 of |
| | | SEQ ID No: 11 |
| LFR4 | FGAGTKLELK | Position 103-112 of |
| | | SEQ ID No: 11 |

118H1

| | | |
|---|---|---|
| V_{H} | EVQLVESGGGLVQPKGSLKLSCAASGFSFNPYAMNWVRQAPGKGLE | SEQ ID No: 15 |
| | WIARIRSKSNNYAAYYADSVKDRFTISRDDSENILYLQMKNLITEDTA | |
| | MYYCVRDYYGTRPSIGMDYWGRGTSVTVSS | |
| HCDR1 | GFSFNPYA | SEQ ID No: 2 |
| HCDR2 | IRSKSNNYAA | SEQ ID No: 16 |
| HCDR3 | VRDYYGTRPSIGMDY | SEQ ID No: 17 |
| HFR1 | EVQLVESGGGLVQPKGSLKLSCAAS | Position 1-25 of |
| | | SEQ ID No: 15 |
| HFR2 | MNWVRQAPGKGLEWIAR | Position 34-50 of |
| | | SEQ ID No: 15 |
| HFR3 | YYADSVKDRFTISRDDSENILYLQMKNLITEDTAMYYC | Position 61-98 of |
| | | SEQ ID No: 15 |
| HFR4 | WGRGTSVTVSS | Position 114-124 of |
| | | SEQ ID No: 15 |
| V_{L} | DIAMTQAAPSVFVTPGESVSISCRSSQSLLHSNGNTYLYWFLQRPGRS | SEQ ID No: 18 |
| | PQLLIYRMSNLASGVPDRFSGSGSGTGFTLRISRVEAEDVGVYYCMQ | |
| | HLEYPFTFGAGTKLELK | |
| LCDR1 | QSLLHSNGNTY | SEQ ID No: 19 |
| LCDR2 | RMS | SEQ ID No: 7 |
| LCDR3 | MQHLEYPFT | SEQ ID No: 14 |
| LFR1 | DIAMTQAAPSVFVTPGESVSISCRSS | Position 1-26 of |
| | | SEQ ID No: 18 |
| LFR2 | LYWFLQRPGRSPQLLIY | Position 38-54 of |
| | | SEQ ID No: 18 |
| LFR3 | NLASGVPDRFSGSGSGTGFTLRISRVEAEDVGVYYC | Position 58-93 of |
| | | SEQ ID No: 18 |
| LFR4 | FGAGTKLELK | Position 103-112 of |
| | | SEQ ID No: 18 |

170G6

| | | |
|---|---|---|
| V_{H} | EVQLQQSGPVLVKPGASVKMSCKASGYTFTDNYMNWVKQSHGKSL | SEQ ID No: 21 |
| | EWIGVINPYNGVTRYNQKFRGKATLTADKSSSTAFMDLNSLTSEDSA | |
| | VYYCSNSLSWGPGTTLTVSS | |
| HCDR1 | GYTFTDNY | SEQ ID No: 22 |
| HCDR2 | INPYNGVT | SEQ ID No: 23 |
| HCDR3 | SNSLS | SEQ ID No: 24 |
| HFR1 | EVQLQQSGPVLVKPGASVKMSCKAS | Position 1-25 of |
| | | SEQ ID No: 21 |
| HFR2 | MNWVKQSHGKSLEWIGV | Position 34-50 of |
| | | SEQ ID No: 21 |
| HFR3 | RYNQKFRGKATLTADKSSSTAFMDLNSLTSEDSAVYYC | Position 59-96 of |
| | | SEQ ID No: 21 |
| HFR4 | WGPGTTLTVSS | Position 102-112 of |
| | | SEQ ID No: 21 |
| V_{L} | DVVMTQSPLTLSVTIGQTASISCKSSQSLLDTDGKTYLNWLLQRPGQS | SEQ ID No: 25 |
| | PKRLIYLVSKLDSGVPDRFTGSGSGTDFTLKISRVEAEDLGVYYCWQG | |
| | IHYPRTFGGGTKLEIK | |
| LCDR1 | QSLLDTDGKTY | SEQ ID No: 26 |
| LCDR2 | LVS | SEQ ID No: 20 |
| LCDR3 | WQGIHYPRT | SEQ ID No: 27 |
| LFR1 | DVVMTQSPLTLSVTIGQTASISCKSS | Position 1-26 of |
| | | SEQ ID No: 25 |
| LFR2 | LNWLLQRPGQSPKRLIY | Position 38-54 of |
| | | SEQ ID No: 25 |
| LFR3 | KLDSGVPDRFTGSGSGTDFTLKISRVEAEDLGVYYC | Position 58-93 of |
| | | SEQ ID No: 25 |
| LFR4 | FGGGTKLEIK | Position 103-112 of |
| | | SEQ ID No: 25 |

172E7

| | | |
|---|---|---|
| V_{H} | EVQLQQSGPVLVKPGASVKMSCKASGYTFTDNYMNWMKQSHGKTL | SEQ ID No: 28 |
| | EWIGVINPYNGVTRYNQKFRDKATLTVDKSSSTAYMDLNSLTSEDSA | |
| | VYYCSNSLSWGPGTSLTVSS | |
| HCDR1 | GYTFTDNY | SEQ ID No: 22 |
| HCDR2 | INPYNGVT | SEQ ID No: 23 |
| HCDR3 | SNSLS | SEQ ID No: 24 |
| HFR1 | EVQLQQSGPVLVKPGASVKMSCKAS | Position 1-25 of |
| | | SEQ ID No: 28 |
| HFR2 | MNWMKQSHGKTLEWIGV | Position 34-50 of |
| | | SEQ ID No: 28 |
| HFR3 | RYNQKFRDKATLTVDKSSSTAYMDLNSLTSEDSAVYYC | Position 59-96 of |
| | | SEQ ID No: 28 |
| HFR4 | WGPGTSLTVSS | Position 102-112 of |
| | | SEQ ID No: 28 |
| V_{L} | DVVMTQTPLTLSVTIGQAASISCKSSQSLVDSDGKTYLNWLLQRPGQS | SEQ ID No: 29 |
| | PKRLIYLVSKLDSGVPDRFTGSGSGTDFTLKISRVEAEDLGVYYCWQG | |
| | IHYPRTFGGGTKLEII | |
| LCDR1 | QSLVDSDGKTY | SEQ ID No: 30 |
| LCDR2 | LVS | SEQ ID No: 20 |
| LCDR3 | WQGIHYPRT | SEQ ID No: 27 |
| LFR1 | DVVMTQTPLTLSVTIGQAASISCKSS | Position 1-26 of |
| | | SEQ ID No: 29 |
| LFR2 | LNWLLQRPGQSPKRLIY | Position 38-54 of |
| | | SEQ ID No: 29 |
| LFR3 | KLDSGVPDRFTGSGSGTDFTLKISRVEAEDLGVYYC | Position 58-93 of |
| | | SEQ ID No: 29 |
| LFR4 | WGRGTSVTVSS | Position 103-112 of |
| | | SEQ ID No: 29 |

84B4

| | | |
|---|---|---|
| V_{H} | EVQLQQSGPVLVKPGASVKMSCKASGFTITDYYFNWVKQSHGKSLE | SEQ ID No: 31 |
| | WIGIINPYNGVARYKQKFKGKATLTVDKSSSTVYLEFSGLTSEDSAVY | |
| | YCVRFVYGTTYDYAMDYWGQGTSVTVSS | |
| HCDR1 | GFTITDYY | SEQ ID No: 32 |
| HCDR2 | INPYNGVA | SEQ ID No: 33 |
| HCDR3 | VRFVYGTTYDYAMDY | SEQ ID No: 34 |
| HFR1 | EVQLQQSGPVLVKPGASVKMSCKAS | Position 1-25 of |
| | | SEQ ID No: 31 |
| HFR2 | FNWVKQSHGKSLEWIGI | Position 34-50 of |
| | | SEQ ID No: 31 |
| HFR3 | RYKQKFKGKATLTVDKSSSTVYLEFSGLTSEDSAVYYC | Position 59-96 of |
| | | SEQ ID No: 31 |
| HFR4 | WGQGTSVTVSS | Position 112-122 of |
| | | SEQ ID No: 31 |
| V_{L} | DVLMTQTPLSLPVSLGDQASISCRSSQSIVHSNGNTYLEWYVQKPGQS | SEQ ID No: 35 |
| | PKLLIYKVSNRFSGVPDRFSGSGSGTDFTLKISRVEAEDLGVYYCFQG | |
| | SHVPPTFGGGTKLEIK | |
| LCDR1 | QSIVHSNGNTY | SEQ ID No: 36 |
| LCDR2 | KVS | SEQ ID No: 37 |
| LCDR3 | FQGSHVPPT | SEQ ID No: 38 |
| LFR1 | DVLMTQTPLSLPVSLGDQASISCRSS | Position 1-26 of |
| | | SEQ ID No: 35 |
| LFR2 | LEWYVQKPGQSPKLLIY | Position 38-54 of |
| | | SEQ ID No: 35 |
| LFR3 | NRFSGVPDRFSGSGSGTDFTLKISRVEAEDLGVYYC | Position 58-93 of |
| | | SEQ ID No: 35 |
| LFR4 | FGGGTKLEIK | Position 103-112 of |
| | | SEQ ID No: 35 |

2P15

| | | |
|---|---|---|
| V_{H} | EVQLVESGGGLVQPKGSLKLSCAASGFSFNAYAMNWVRQAPGKGLE | SEQ ID No: 39 |
| | WVARMRSKSNYYATYYADSVKDRFTISRDDSESMLYLQMNNLKTED | |
| | TAMYYCVRQTYGSKDYAMDYWGQGTSVTVSS | |
| HCDR1 | GFSFNAYA | SEQ ID No: 40 |
| HCDR2 | MRSKSNYYAT | SEQ ID No: 41 |
| HCDR3 | VRQTYGSKDYAMDY | SEQ ID No: 42 |
| HFR1 | EVQLVESGGGLVQPKGSLKLSCAAS | Position 1-25 of |
| | | SEQ ID No: 39 |
| HFR2 | MNWVRQAPGKGLEWVAR | Position 34-50 of |
| | | SEQ ID No: 39 |
| HFR3 | YYADSVKDRFTISRDDSESMLYLQMNNLKTEDTAMYYC | Position 59-96 of |
| | | SEQ ID No: 39 |
| HFR4 | WGQGTSVTVSS | Position 112-122 of |
| | | SEQ ID No: 39 |
| V_{L} | DVVMTQTPLSLPVSLGDQASISCRSSQSLVHSNGNTYFHWYLQKPGQ | SEQ ID No: 43 |
| | SPKLLIYKVSNRFSGVPDRFSGSGSGTDFTLKISRVEAEDLGVYFCSQS | |
| | THVPFTFGSGTKLEIK | |
| LCDR1 | QSLVHSNGNTY | SEQ ID No: 44 |
| LCDR2 | KVS | SEQ ID No: 37 |
| LCDR3 | SQSTHVPFT | SEQ ID No: 45 |
| LFR1 | DVVMTQTPLSLPVSLGDQASISCRSS | Position 1-26 of |
| | | SEQ ID No: 43 |
| LFR2 | FHWYLQKPGQSPKLLIY | Position 38-54 of |
| | | SEQ ID No: 43 |
| LFR3 | NRFSGVPDRFSGSGSGTDFTLKISRVEAEDLGVYFC | Position 58-93 of |
| | | SEQ ID No: 43 |
| LFR4 | FGSGTKLEIK | Position 103-112 of |
| | | SEQ ID No: 43 |

3C11

| | | |
|---|---|---|
| V_{H} | EFQLQQSGPELVKPGASVKISCKTSGYSFTDYNINWVKQSNGKSLEWI | SEQ ID No: 46 |
| | GLINPNHGTTKSNQKFKGKATLTVDQSSSTAYMQLSSLTSEDSAVYY | |
| | CARHDYDGAYWGQGTLVTVSA | |
| HCDR1 | GYSFTDYN | SEQ ID No: 47 |
| HCDR2 | INPNHGTT | SEQ ID No: 48 |
| HCDR3 | ARHDYDGAY | SEQ ID No: 49 |
| HFR1 | EFQLQQSGPELVKPGASVKISCKTS | Position 1-25 of |
| | | SEQ ID No: 46 |
| HFR2 | INWVKQSNGKSLEWIGL | Position 34-50 of |
| | | SEQ ID No: 46 |
| HFR3 | KSNQKFKGKATLTVDQSSSTAYMQLSSLTSEDSAVYYC | Position 59-96 of |
| | | SEQ ID No: 46 |
| HFR4 | WGQGTLVTVSA | Position 106-116 of |
| | | SEQ ID No: 46 |
| V_{L} | DVVMTQTPLSLPVSLGDQASISCRSSQSLIHSNGNTYLHWYLQKPGQS | SEQ ID No: 50 |
| | PKLLIYKISNRFSGVPDRFSGSGSGTDFTLKISRVEAEDLGVYFCSQST | |
| | RAPWTFGGGTKLEIK | |
| LCDR1 | QSLIHSNGNTY | SEQ ID No: 51 |
| LCDR2 | KIS | SEQ ID No: 52 |
| LCDR3 | SQSTRAPWT | SEQ ID No: 53 |
| LFR1 | DVVMTQTPLSLPVSLGDQASISCRSS | Position 1-26 of |
| | | SEQ ID No: 50 |
| LFR2 | LHWYLQKPGQSPKLLIY | Position 38-54 of |
| | | SEQ ID No: 50 |
| LFR3 | NRFSGVPDRFSGSGSGTDFTLKISRVEAEDLGVYFC | Position 58-93 of |
| | | SEQ ID No: 50 |
| LFR4 | FGGGTKLEIK | Position 103-112 of |
| | | SEQ ID No: 50 |

3O20

| | | |
|---|---|---|
| V_{H} | EVQLVESGGGLVQPKGSLKLSCATSGFSFNAYAMNWVRQAPGKGLE | SEQ ID No: 54 |
| | WVGRIRSKSNNYATYYADSVKGRFTISRDDSKTILYLQMNTLKTEDT | |
| | AIYYCVRGGYHGNSAYFDVWGTGTSVTVSS | |
| HCDR1 | GFSFNAYA | SEQ ID No: 40 |
| HCDR2 | IRSKSNNYAT | SEQ ID No: 3 |
| HCDR3 | VRGGYHGNSAYFDV | SEQ ID No: 55 |
| HFR1 | EVQLVESGGGLVQPKGSLKLSCATS | Position 1-25 of |
| | | SEQ ID No: 54 |
| HFR2 | MNWVRQAPGKGLEWVGR | Position 34-50 of |
| | | SEQ ID No: 54 |
| HFR3 | YYADSVKGRFTISRDDSKTILYLQMNTLKTEDTAIYYC | Position 61-98 of |
| | | SEQ ID No: 54 |
| HFR4 | WGTGTSVTVSS | Position 113-123 of |
| | | SEQ ID No: 54 |
| V_{L} | DIVMTQAAPSVPVTPGESVSISCRSSKSLLHSNGNTYLYWFLQRPGQS | SEQ ID No: 56 |
| | PQLLIYRMSNLASGVPDRFSGSGSGTAFTLRISRVEAEDVGVYYCLQH | |
| | LEYPFTFGSGTKLEIK | |
| LCDR1 | KSLLHSNGNTY | SEQ ID No: 6 |
| LCDR2 | RMS | SEQ ID No: 7 |
| LCDR3 | LQHLEYPFT | SEQ ID No: 8 |
| LFR1 | DIVMTQAAPSVPVTPGESVSISCRSS | Position 1-26 of |
| | | SEQ ID No: 56 |
| LFR2 | LYWFLQRPGQSPQLLIY | Position 38-54 of |
| | | SEQ ID No: 56 |
| LFR3 | NLASGVPDRFSGSGSGTAFTLRISRVEAEDVGVYYC | Position 58-93 of |
| | | SEQ ID No: 56 |
| LFR4 | FGSGTKLEIK | Position 103-112 of |
| | | SEQ ID No: 56 |

4M13

| | | |
|---|---|---|
| V_{H} | EVQLVESGGGLVQPKGSLKLSCAASGFSFNTYAMNWVRQAPGKGLE | SEQ ID No: 57 |
| | WVARMRSKSNNYATYYADSVKDRFTISRDDSESMLYLQMNNLKTED | |
| | TAMYYCVRGKDTSGSYAMDYWGQGTSVSVSS | |
| HCDR1 | GFSFNTYA | SEQ ID No: 58 |
| HCDR2 | MRSKSNNYAT | SEQ ID No: 59 |
| HCDR3 | VRGKDTSGSYAMDY | SEQ ID No: 60 |
| HFR1 | EVQLVESGGGLVQPKGSLKLSCAAS | Position 1-25 of |
| | | SEQ ID No: 57 |
| HFR2 | MNWVRQAPGKGLEWVAR | Position 34-50 of |
| | | SEQ ID No: 57 |
| HFR3 | YYADSVKDRFTISRDDSESMLYLQMNNLKTEDTAMYYC | Position 61-98 of |
| | | SEQ ID No: 57 |
| HFR4 | WGQGTSVSVSS | Position 113-123 of |
| | | SEQ ID No: 57 |
| V_{L} | DIVMTQAAPSVPVTPGESVSISCRSSKSLLHSNGNTYLYWFLQRPGQS | SEQ ID No: 61 |
| | PHLLIYRMSNLASGVPDRFSGSGSGTAFTLRISKVETEDVGVYYCMQ | |
| | HLEYPFTFGSGTKLEIK | |
| LCDR1 | KSLLHSNGNTY | SEQ ID No: 6 |
| LCDR2 | RMS | SEQ ID No: 7 |
| LCDR3 | MQHLEYPFT | SEQ ID No: 14 |
| LFR1 | DIVMTQAAPSVPVTPGESVSISCRSS | Position 1-26 of |
| | | SEQ ID No: 61 |
| LFR2 | LYWFLQRPGQSPHLLIY | Position 38-54 of |
| | | SEQ ID No: 61 |
| LFR3 | NLASGVPDRFSGSGSGTAFTLRISKVETEDVGVYYC | Position 58-93 of |
| | | SEQ ID No: 61 |
| LFR4 | FGSGTKLEIK | Position 103-112 of |
| | | SEQ ID No: 61 |

2L15

| | | |
|---|---|---|
| V_{H} | EVQLVESGGGLVQPKGSLKLSCAASGFSFNTYAMNWVRQAPGKGLE | SEQ ID No: 62 |
| | WVARIRSKSNNYATYYADSVKDRFTISRADSESMLYLQMNNLKTED | |
| | TAMYYCVRGKDISVSYAMDYWGQGTSVTVSS | |
| HCDR1 | GFSFNTYA | SEQ ID No: 58 |
| HCDR2 | IRSKSNNYAT | SEQ ID No: 3 |
| HCDR3 | VRGKDISVSYAMDY | SEQ ID No: 63 |
| HFR1 | EVQLVESGGGLVQPKGSLKLSCAAS | Position 1-25 of |
| | | SEQ ID No: 62 |
| HFR2 | MNWVRQAPGKGLEWVAR | Position 34-50 of |
| | | SEQ ID No: 62 |
| HFR3 | YYADSVKDRFTISRADSESMLYLQMNNLKTEDTAMYYC | Position 61-98 of |
| | | SEQ ID No: 62 |
| HFR4 | WGQGTSVTVSS | Position 113-123 of |
| | | SEQ ID No: 62 |
| V_{L} | DIVMTQAAPSVPVTPGESVSISCRSSKSLQHSNGNTYLYWFLQRPGQS | SEQ ID No: 64 |
| | PQLLIYRMSNLASGVPDRFSGSGSGTAFTLRISRVETEDVGVYYCMQH | |
| | LEYPFTFGSGTKLEIK | |
| LCDR1 | KSLQHSNGNTY | SEQ ID No: 65 |
| LCDR2 | RMS | SEQ ID No: 7 |
| LCDR3 | MQHLEYPFT | SEQ ID No: 14 |
| LFR1 | DIVMTQAAPSVPVTPGESVSISCRSS | Position 1-26 of |
| | | SEQ ID No: 64 |
| LFR2 | LYWFLQRPGQSPQLLIY | Position 38-54 of |
| | | SEQ ID No: 64 |
| LFR3 | NLASGVPDRFSGSGSGTAFTLRISRVETEDVGVYYC | Position 58-93 of |
| | | SEQ ID No: 64 |
| LFR4 | FGSGTKLEIK | Position 103-112 of |
| | | SEQ ID No: 64 |

4D24

| | | |
|---|---|---|
| V_{H} | EVQLVESGGGLVQPKGSLKLSCAASGFSFNPYAMNWVRQSPGKGLE | SEQ ID No: 66 |
| | WVARIRSKSNNYATYYADSVKDRFTISRDDSESMLYLQMNNLKTED | |
| | TAMYYCVRQGWVKRYFDVWGTGTTVTVSS | |
| HCDR1 | GFSFNPYA | SEQ ID No: 2 |
| HCDR2 | IRSKSNNYAT | SEQ ID No: 3 |
| HCDR3 | VRQGWVKRYFDV | SEQ ID No: 67 |
| HFR1 | EVQLVESGGGLVQPKGSLKLSCAAS | Position 1-25 of |
| | | SEQ ID No: 66 |
| HFR2 | MNWVRQSPGKGLEWVAR | Position 34-50 of |
| | | SEQ ID No: 66 |
| HFR3 | YYADSVKDRFTISRDDSESMLYLQMNNLKTEDTAMYYC | Position 61-98 of |
| | | SEQ ID No: 66 |
| HFR4 | WGTGTTVTVSS | Position 111-121 of |
| | | SEQ ID No: 66 |
| V_{L} | GNVLTQSPAIMSASPGEKVTMTCSASSSGTYMHWYQQKSTTSPKLWI | SEQ ID No: 68 |
| | YDTSKLASGVPGRFSGSGSGNSYSLTISSMEAEDIATYYCFQGSGYPL | |
| | TFGSGTKLEIK | |
| LCDR1 | SSGTY | SEQ ID No: 69 |
| LCDR2 | DTS | SEQ ID No: 70 |
| LCDR3 | FQGSGYPLT | SEQ ID No: 13 |
| LFR1 | GNVLTQSPAIMSASPGEKVTMTCSAS | Position 1-26 of |
| | | SEQ ID No: 68 |
| LFR2 | MHWYQQKSTTSPKLWIY | Position 32-48 of |
| | | SEQ ID No: 68 |
| LFR3 | KLASGVPGRFSGSGSGNSYSLTISSMEAEDIATYYC | Position 52-87 of |
| | | SEQ ID No: 68 |
| LFR4 | FGSGTKLEIK | Position 97-106 of |
| | | SEQ ID No: 68 |

1G17

| | | |
|---|---|---|
| V_{H} | QVQLQQSGAELVRPGASVTLSCKASGYTFADYEMHWVKQTPVLGLE | SEQ ID No: 71 |
| | WIGAIDPETGRTAYNQKFKFKATLTADRSSSTAYMELRSLTSEDSAVY | |
| | YCTRRAYWGGWGQGTTLTVSS | |
| HCDR1 | GYTFADYE | SEQ ID No: 72 |
| HCDR2 | IDPETGRT | SEQ ID No: 73 |
| HCDR3 | TRRAYWGG | SEQ ID No: 74 |
| HFR1 | QVQLQQSGAELVRPGASVTLSCKAS | Position 1-25 of |
| | | SEQ ID No: 71 |
| HFR2 | MHWVKQTPVLGLEWIGA | Position 34-50 of |
| | | SEQ ID No: 71 |
| HFR3 | AYNQKFKFKATLTADRSSSTAYMELRSLTSEDSAVYYC | Position 61-98 of |
| | | SEQ ID No: 71 |
| HFR4 | WGQGTTLTVSS | Position 111-121 of |
| | | SEQ ID No: 71 |
| V_{L} | DVVMTQTPLSLPVSLGDQASISCRSSQSLVHSSGNTYLHWYLQKPGQ | SEQ ID No: 75 |
| | SPKLLIYKVSNRFSGVPDRFSGSGSGTDFTLKISRVEAEDLGVYFCSQS | |
| | THVPYTFGGGTKLEIK | |
| LCDR1 | QSLVHSSGNTY | SEQ ID No: 76 |
| LCDR2 | KVS | SEQ ID No: 37 |
| LCDR3 | SQSTHVPYT | SEQ ID No: 77 |
| LFR1 | DVVMTQTPLSLPVSLGDQASISCRSS | Position 1-26 of |
| | | SEQ ID No: 75 |
| LFR2 | LHWYLQKPGQSPKLLIY | Position 32-48 of |
| | | SEQ ID No: 75 |
| LFR3 | NRFSGVPDRFSGSGSGTDFTLKISRVEAEDLGVYFC | Position 52-87 of |
| | | SEQ ID No: 75 |
| LFR4 | FGGGTKLEIK | Position 97-106 of |
| | | SEQ ID No: 75 |

3F11

| | | |
|---|---|---|
| V_{H} | EVQLVESGGGLVQPKGSLKLSCAASGFSFNTYAMNWVRQAPGKGLE | SEQ ID No: 78 |
| | WVARIRSKSNYYATYYADSVKDRFTISRDDSESMLYLQMNNLKTED | |
| | TAMYYCVRGRELGDYYAMDYWGQGTSVTVSS | |
| HCDR1 | GFSFNTYA | SEQ ID No: 58 |
| HCDR2 | IRSKSNYYAT | SEQ ID No: 79 |
| HCDR3 | VRGRELGDYYAMDY | SEQ ID No: 80 |
| HFR1 | EVQLVESGGGLVQPKGSLKLSCAAS | Position 1-25 of |
| | | SEQ ID No: 78 |
| HFR2 | MNWVRQAPGKGLEWVAR | Position 34-50 of |
| | | SEQ ID No: 78 |
| HFR3 | YYADSVKDRFTISRDDSESMLYLQMNNLKTEDTAMYYC | Position 61-98 of |
| | | SEQ ID No: 78 |
| HFR4 | WGQGTSVTVSS | Position 111-121 of |
| | | SEQ ID No: 78 |
| V_{L} | DIVMTQAAPSVPVTPGESVSISCRSSKSLLHSNGNTYLYWFLQRPGQS | SEQ ID No: 81 |
| | PQLLIYRMSNLASGVPNRFSGSGSGTAFTLRISRVEAEDVGVYYCMQ | |
| | HLEYPFTFGSGTKLEIK | |
| LCDR1 | KSLLHSNGNTY | SEQ ID No: 6 |
| LCDR2 | RMS | SEQ ID No: 7 |
| LCDR3 | MQHLEYPFT | SEQ ID No: 14 |
| LFR1 | DIVMTQAAPSVPVTPGESVSISCRSS | Position 1-26 of |
| | | SEQ ID No: 81 |
| LFR2 | LYWFLQRPGQSPQLLIY | Position 32-48 of |
| | | SEQ ID No: 81 |
| LFR3 | NLASGVPNRFSGSGSGTAFTLRISRVEAEDVGVYYC | Position 52-87 of |
| | | SEQ ID No: 81 |
| LFR4 | FGSGTKLEIK | Position 97-106 of |
| | | SEQ ID No: 81 |

3F6

| | | |
|---|---|---|
| V_{H} | EVQFVESGGGLVQPKGSLKLSCAASGFSFNTYAMNWVRQAPGKGLE | SEQ ID No: 82 |
| | WVARIRTKSNNYATHYADSVKDRFIVSRDDSENILYLQMNNLKTEDT | |
| | GMYYCVRGGNGIYGRNAMDNWGQGTSVTVSS | |
| HCDR1 | GFSFNTYA | SEQ ID No: 58 |
| HCDR2 | IRTKSNNYAT | SEQ ID No: 83 |
| HCDR3 | VRGGNGIYGRNAMDN | SEQ ID No: 84 |
| HFR1 | EVQFVESGGGLVQPKGSLKLSCAAS | Position 1-25 of |
| | | SEQ ID No: 82 |
| HFR2 | MNWVRQAPGKGLEWVAR | Position 34-50 of |
| | | SEQ ID No: 82 |
| HFR3 | HYADSVKDRFIVSRDDSENILYLQMNNLKTEDTGMYYC | Position 61-98 of |
| | | SEQ ID No: 82 |
| HFR4 | WGQGTSVTVSS | Position 111-121 of |
| | | SEQ ID No: 82 |
| V_{L} | DIVMTQAAPSVPVSPGESVSISCRSSKSLLHSSGNTYLYWFLQRPGQSP | SEQ ID No: 85 |
| | QLLIYRMSNLASGVPDRFSGSGSGTAFTLRISRVEAEDVGVYYCMQH | |
| | LEYPFTFGGGTKLEIK | |
| LCDR1 | KSLLHSSGNTY | SEQ ID No: 86 |
| LCDR2 | RMS | SEQ ID No: 7 |
| LCDR3 | MQHLEYPFT | SEQ ID No: 14 |
| LFR1 | DIVMTQAAPSVPVSPGESVSISCRSS | Position 1-26 of |
| | | SEQ ID No: 85 |
| LFR2 | LYWFLQRPGQSPQLLIY | Position 32-48 of |
| | | SEQ ID No: 85 |
| LFR3 | NLASGVPDRFSGSGSGTAFTLRISRVEAEDVGVYYC | Position 52-87 of |
| | | SEQ ID No: 85 |
| LFR4 | FGGGTKLEIK | Position 97-106 of |
| | | SEQ ID No: 85 |

4G19

| | | |
|---|---|---|
| V_{H} | EVQFVESGGGLVQPKGSLKLSCAASGFSFNTYAMNWVRQAPGKGLE | SEQ ID No: 87 |
| | WVARIRTKSNNYATHYADSVTDRFIVSRDDSESMTYLQMNNLKTED | |
| | TGMYYCVRGGNGIYGRNTMDNWGQGTSVTVSS | |
| HCDR1 | GFSFNTYA | SEQ ID No: 58 |
| HCDR2 | IRTKSNNYAT | SEQ ID No: 83 |
| HCDR3 | VRGGNGIYGRNTMDN | SEQ ID No: 88 |
| HFR1 | EVQFVESGGGLVQPKGSLKLSCAAS | Position 1-25 of |
| | | SEQ ID No: 87 |
| HFR2 | MNWVRQAPGKGLEWVAR | Position 34-50 of |
| | | SEQ ID No: 87 |
| HFR3 | HYADSVTDRFIVSRDDSESMTYLQMNNLKTEDTGMYYC | Position 61-98 of |
| | | SEQ ID No: 87 |
| HFR4 | WGQGTSVTVSS | Position 111-121 of |
| | | SEQ ID No: 87 |
| V_{L} | DIVMTQTAPSVPVSPGESVSISCRSSKSLLHSSGNTYLYWFLQRPGQSP | SEQ ID No: 89 |
| | QLLIYRMSNLASGVPDRFSGSGSGTAFTLRISRVEAEDVGVYYCMQH | |
| | LEYPFTFGGGTKLEIQ | |
| LCDR1 | KSLLHSSGNTY | SEQ ID No: 86 |
| LCDR2 | RMS | SEQ ID No: 7 |
| LCDR3 | MQHLEYPFT | SEQ ID No: 14 |
| LFR1 | DIVMTQTAPSVPVSPGESVSISCRSS | Position 1-26 of |
| | | SEQ ID No: 89 |
| LFR2 | LYWFLQRPGQSPQLLIY | Position 32-48 of |
| | | SEQ ID No: 89 |
| LFR3 | NLASGVPDRFSGSGSGTAFTLRISRVEAEDVGVYYC | Position 52-87 of |
| | | SEQ ID No: 89 |
| LFR4 | FGGGTKLEIQ | Position 97-106 of |
| | | SEQ ID No: 89 |

TABLE 2
Sequence IDs of VH, VL and CDRs of antibodies.

| | Region (SEQ ID No.) | | | | | | | |
|---|---|---|---|---|---|---|---|---|
| Antibody | V_{H} | | | | V_{L} | | | |
| (clone) | V_{H} | CDR1 | CDR2 | CDR3 | V_{L} | CDR1 | CDR2 | CDR3 |
| 101E4 | 1 | 2 | 3 | 4 | 5 | 6 | 7 | 8 |
| 103G4 | 9 | 2 | 3 | 10 | 11 | 12 | 7 | 14 |
| 118H1 | 15 | 2 | 16 | 17 | 18 | 19 | 7 | 14 |
| 170G6 | 21 | 22 | 23 | 24 | 25 | 26 | 20 | 27 |
| 172E7 | 28 | 22 | 23 | 24 | 29 | 30 | 20 | 27 |
| 84B4 | 31 | 32 | 33 | 34 | 35 | 36 | 37 | 38 |
| 2P15 | 39 | 40 | 41 | 42 | 43 | 44 | 37 | 45 |
| 3C11 | 46 | 47 | 48 | 49 | 50 | 51 | 52 | 53 |
| 3O20 | 54 | 40 | 3 | 55 | 56 | 6 | 7 | 8 |
| 4M13 | 57 | 58 | 59 | 60 | 61 | 6 | 7 | 14 |
| 2L15 | 62 | 58 | 3 | 63 | 64 | 65 | 7 | 14 |
| 4D24 | 66 | 2 | 3 | 67 | 68 | 69 | 70 | 13 |
| 1G17 | 71 | 72 | 73 | 74 | 75 | 76 | 37 | 77 |
| 3F11 | 78 | 58 | 79 | 80 | 81 | 6 | 7 | 14 |
| 3F6 | 82 | 58 | 83 | 84 | 85 | 86 | 7 | 14 |
| 4G19 | 87 | 58 | 83 | 88 | 89 | 86 | 7 | 14 |

### Example 2. Binding Affinity of Anti-CCR8 Antibodies Against CCR8 Expressing Cells

The affinity of anti-CCR8 antibodies was evaluated by FACS. 293F-CCR8, which had high level of CCR8 expressed on the cell surface, was used in the binding test. Affinity analyses were generally carried out as follows. Approximately 5×10⁵ 293F-CCR8 cells per sample were prepared and blocked with Human BD Fc Block. The test antibodies were diluted with PBS/0.5 BSA (1:3 series dilution from 225 μg/mL to 0.00381 μg/mL). The transfected cells along with parent non-transfected cells were distributed into 96-well round-bottom polystyrene plates and incubated with the diluted antibodies on ice for 20-30 minutes. Next, the samples were washed with PBS/0.5 BSA and the cells were centrifuged. The pelleted cell samples were incubated with the second antibody of anti-mouse IgG-FITC-at 1:300 dilution in 100 μL PBS/0.5% BSA on ice for 30 minutes, and then washed with PBS/0.5% BSA, followed by pelleting the cells. The cell pellets were re-suspended in PBS/0.5% BSA for reading, and samples were analyzed using CYTOFLEX (Beckman).

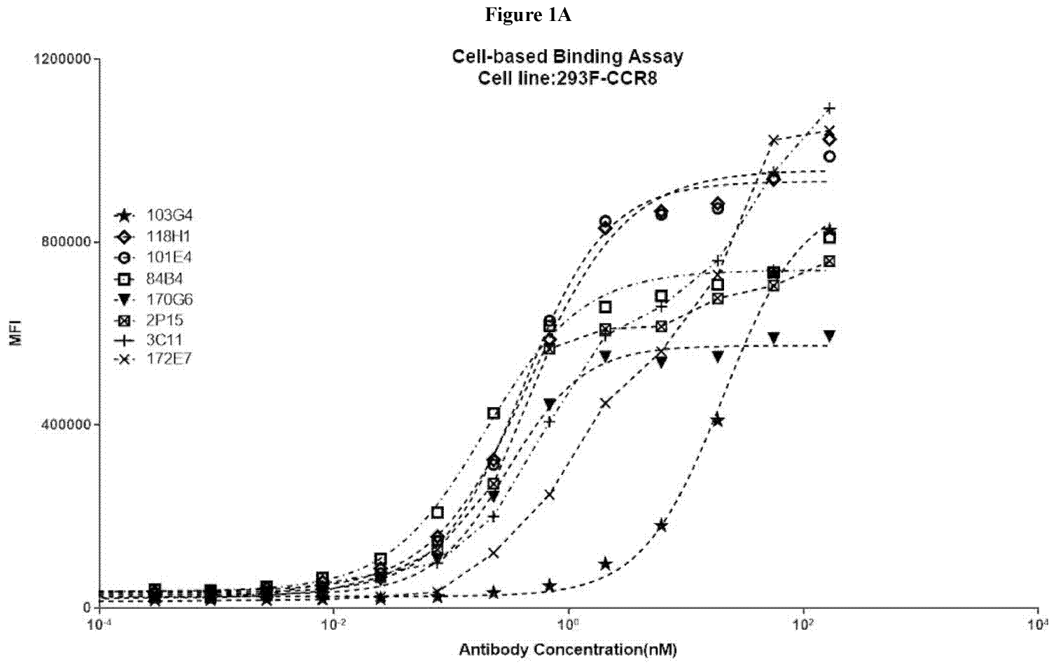

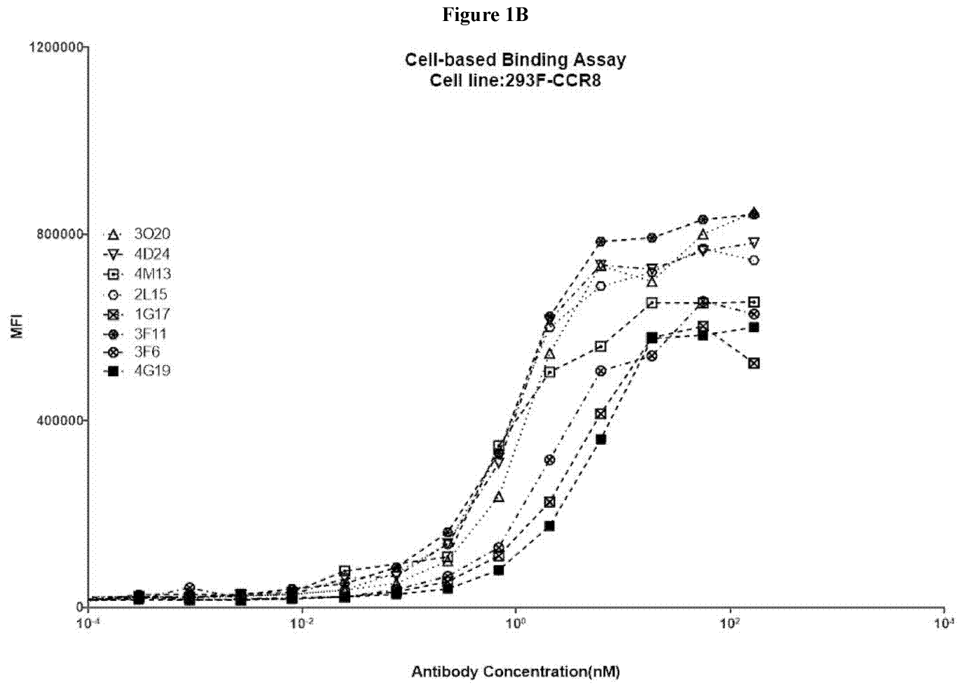

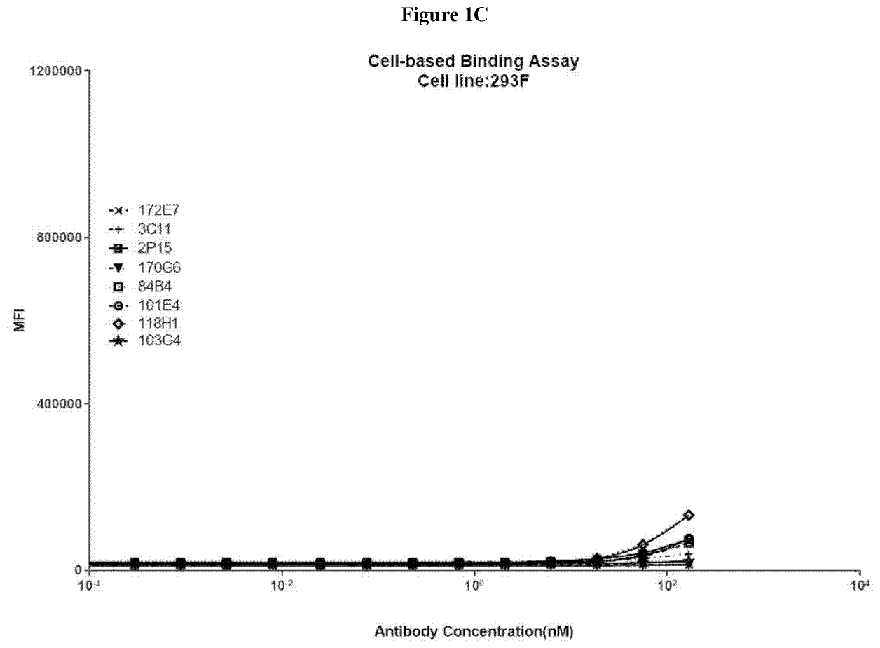

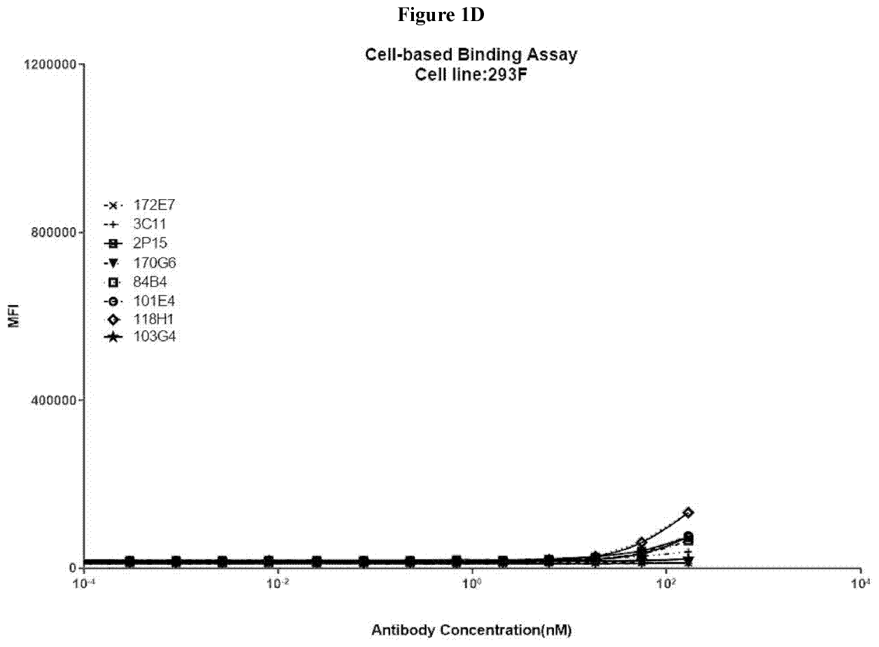

The results showed that these antibodies bound to the human CCR8 overexpressing 293F cells, but didn't bind to cells without CCR8 overexpression as shown in FIG. 1. FIGS. 1A and 1B demonstrates that these antibodies have binding ability to CCR8 overexpressing 293F cells (293F-CCR8), but not the 293F parent cells (FIG. 1C and 1D). The EC50s of the clones showing binding ability are summarized in Table 3:

TABLE 3
EC50s of the clones showing binding ability

| Clones | 103G4 | 118H1 | 101E4 | 84B4 | 170G6 | 172E7 | 2P15 | 3C11 |
|---|---|---|---|---|---|---|---|---|
| EC50 (nM) | 20.56 | 0.4664 | 0.4088 | 0.195 | 0.3021 | 6.989 | 0.3052 | 4.254 |

| Clones | 3O20 | 4M13 | 2L15 | 4D24 | 1G17 | 3F11 | 3F6 | 4G19 |
|---|---|---|---|---|---|---|---|---|
| EC50 (nM) | 1.293 | 0.775 | 0.8257 | 0.888 | 2.939 | 0.9572 | 2.293 | 4.487 |

### Example 3. Anti-CCR8 Antibodies Block CCL1-CCR8 Signal

In order to determine which of the antibodies described above can block CCL1-CCR8 signal, Tango-CCR8-Gal4-CHO-K1 cells were constructed. Cells were pelleted and re-suspended at 1×10⁴ cells/70 μL/well, the cells were distributed into 96 well plate, incubated in starving medium (F12K, 1% FBS, 1% penicillin-streptomycin) in 5% CO₂ at 37° C. for 6 hours, the test antibodies were added and incubated for 1 hour, and then CCL1 (R&D, Catalog Number: 272-I) was added and incubated in 5% CO₂ at 37° C. for 24 hours.

After overnight culture, the cells were incubated with ONE-Glo working reagent, at room temperature in dark for 10 minutes, and then the relative luminescence units (RLU) of each sample were measured using microplate luminescence reader at 560 nm and recorded.

The antibodies from the clones such as 101E4, 170G6, 3O20, 4M13, 4D24, 3F11, 3F6 shown strong CCL1-CCR8 signaling block. The signal blocking ability for the antibodies from each clone is shown in the Table 4.

TABLE 4
Tango assay result of the antibodies.

| Clone | Tango IC₅₀ (nM) |
|---|---|
| 103G4 | 0.83 |
| 118H1 | 1.43 |
| 101E4 | 0.27 |
| 84B4 | 1.86 |
| 172E7 | 2.01 |
| 2P15 | 1.87 |
| 3C11 | 4.08 |
| 170G6 | 0.33 |
| 3O20 | 0.45 |
| 4M13 | 0.47 |
| 2L15 | 1.23 |
| 4D24 | 0.67 |
| 1G17 | 1.82 |
| 3F11 | 0.27 |
| 3F6 | 0.51 |
| 4G19 | 2.61 |

### Example 4. Anti-Tumor Efficacy Testing of Anti-CCR8 Antibodies

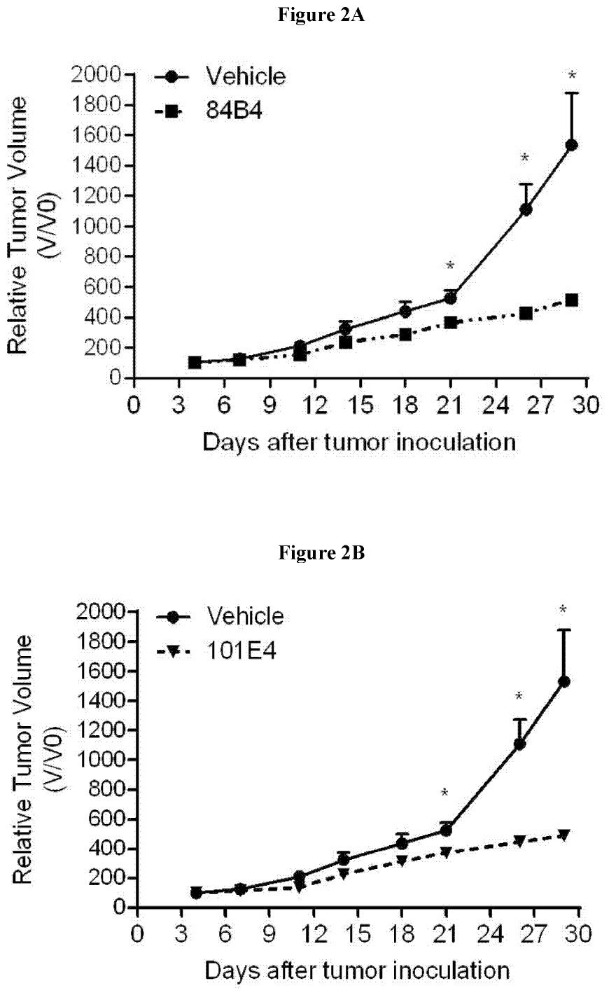

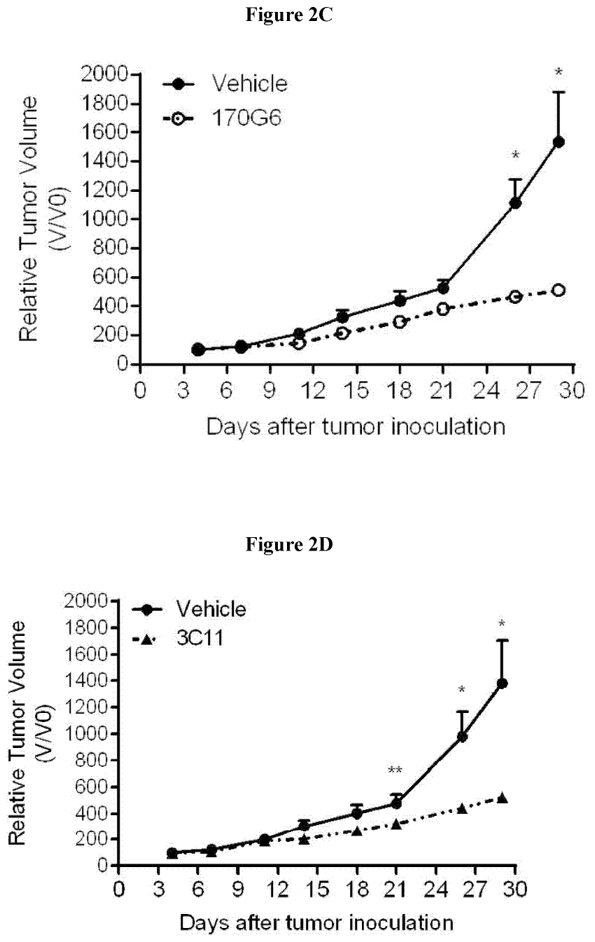

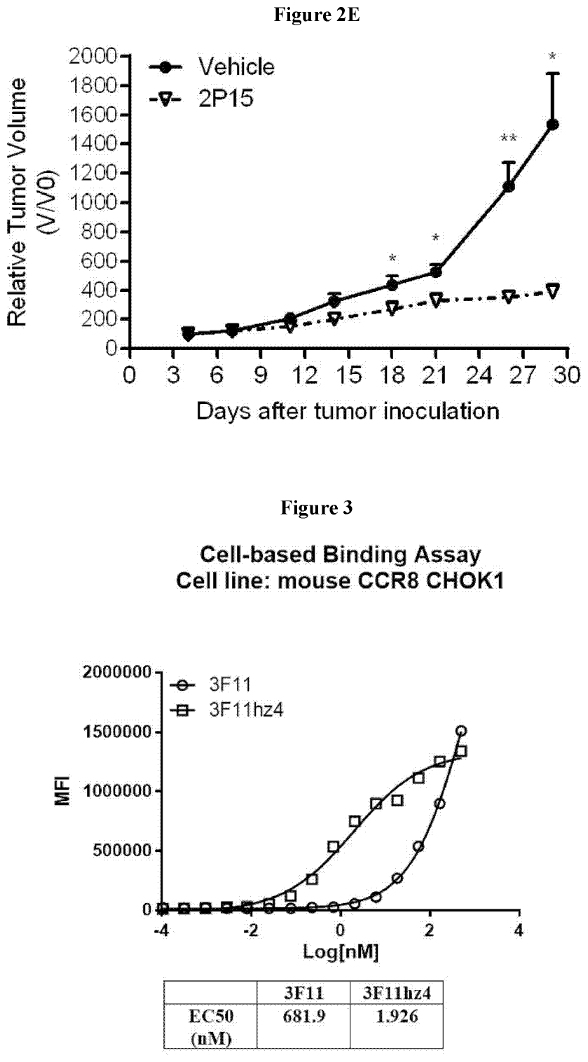

In vivo anti-tumor efficacy was evaluated in humanized mice reconstituted with human peripheral blood mononuclear cells (hu-PBMCs, Milestone Biotechnologies). To facilitate the study, an MDA-MB-231 mouse xenograft model was established. Human breast cancer MDA-MB-231 cells (5×10⁶/mouse) were subcutaneously inoculated into the skin of the front and right back of female NCG mice. When the average size of the transplanted tumor reached 80-100 mm³, tumor-bearing mice with similar tumor size were selected, and randomly grouped and injected with 2×10⁶ hu-PBMCs derived from healthy donors via the tail vein. One hour later, CCR8 antibodies were intraperitoneally injected into mice once a week. The size of the tumor was measured twice a week with a caliper, and the size of the tumor is expressed by volume (mm³), and the formula is: V=0.5a×b². (a and b represent the long diameter and short diameter of the tumor, respectively). As shown FIG. 2 (A-E), the antibodies from the clones of 84B4, 101E4, 170G6, 3C11 and 2P15 had significant anti-tumor effects with P≤0.001.

The results are shown in FIG. 2. According to the tumor growth curve, tumor growth was significantly inhibited in the mouse group injected with anti-CCR8 antibodies, compared with the vehicle treatment group (P<0.001).

### Example 5. The Binding Ability of the Anti-CCR8 Antibody to Cynomolgus CCR8

293F cells were transfected with lentiviral vectors encoding cynomolgus CCR8 by polybrene (8 g/mL), selected in media containing puromycin (2 μg/mL), and tested for the expression of cynomolgus CCR8 by FACS.

Antibodies were studied for specific binding to cynomolgus CCR8 by flow cytometry using cynomolgus CCR8-overexpressing and parental 293F cell line. The results shows that the antibodies 84B4, 170G6, 172E7, 3C11, 3O20, 1G17, 3F11 and 4G19 bound 293F cells expressing cynomolgus CCR8, but did not bind 293F cells.

### Example 6. Humanization of Antibodies 3F11, 3O20 and 1G17

Antibodies 3F11, 3O20 and 1G17 were humanized by grafting the CDRs of lead antibodies into selected human IgG germline frameworks. Human germline IGHV3-73*01, IGKV2-28*01, IGHV1-46*01 and IGKV2-30*02 were selected based on sequence similarity within both frameworks (FR). To maintain canonical loop structure and chain interface, certain residues in human germline frameworks were back mutated to corresponding mouse residues (Table 5).

In silico prediction implied high risk sequence liabilities in CDRs of 3F11. For example, there is an NG motif in CDR-L1 region of 3F11. One liability mutation at N33 position in the light chain was evaluated to see if the potential deamidation site in the VL could be removed without affecting activity.

Humanization of 3F11, 3O20 and 1G17 resulted in monoclonal antibodies 3F11hz0, 3F11hz1, 3F11hz2, 3F11hz3, 3F11hz4, 3F11hz5, and 3F11hz6; 3O20hz0, 3O20hz1, 3O20hz2, and 3O20hz3; and 1G17hz0, 1G17hz1, 1G17hz2 and 1G17hz3.

TABLE 5
Humanization of 3F11, 3O20 and 1G17
3F11hz0

| | | |
|---|---|---|
| V_{H} | EVQLVESGGGLVQPKGSLKLSCAASGFSFNTYAMNWVRQAPGKGLE | SEQ ID No: 78 |
| | WVARIRSKSNYYATYYADSVKDRFTISRDDSESMLYLQMNNLKTED | |
| | TAMYYCVRGRELGDYYAMDYWGQGTSVTVSS | |
| V_{L} | DIVMTQAAPSVPVTPGESVSISCRSSKSLLHSNGNTYLYWFLQRPGQS | SEQ ID No: 81 |
| | PQLLIYRMSNLASGVPNRFSGSGSGTAFTLRISRVEAEDVGVYYCMQ | |
| | HLEYPFTFGSGTKLEIK | |

3F11hz1

| | | |
|---|---|---|
| V_{H} | EVQLVESGGGLVQPGGSLKLSCAASGFSFNTYAMHWVRQASGKGLE | SEQ ID No: 90 |
| | WVGRIRSKSNYYATYYAASVKGRFTISRDDSKNTAYLQMNSLKTEDT | |
| | AVYYCTRGRELGDYYAMDYWGQGTLVTVSS | |
| V_{L} | DIVMTQSPLSLPVTPGEPASISCRSSKSLLHSNGNTYLDWYLQKPGQSP | SEQ ID No: 91 |
| | QLLIYRMSNRASGVPDRFSGSGSGTDFTLKISRVEAEDVGVYYCMQH | |
| | LEYPFTFGQGTKLEIK | |

3F11hz2

| | | |
|---|---|---|
| V_{H} | EVQLVESGGGLVQPGGSLKLSCAASGFSFNTYAMNWVRQASGKGLE | SEQ ID No: 92 |
| | WVGRIRSKSNYYATYYAASVKGRFTISRDDSKNTAYLQMNSLKTEDT | |
| | AVYYCVRGRELGDYYAMDYWGQGTLVTVSS | |
| V_{L} | DIVMTQSPLSLPVTPGEPASISCRSSKSLLHSNGNTYLDWFLQKPGQSP | SEQ ID No: 93 |
| | QLLIYRMSNRASGVPDRFSGSGSGTDFTLKISRVEAEDVGVYYCMQH | |
| | LEYPFTFGQGTKLEIK | |

3F11hz3

| | | |
|---|---|---|
| V_{H} | EVQLVESGGGLVQPGGSLKLSCAASGFTFSTYAMNWVRQASGKGLE | SEQ ID No: 94 |
| | WVGRIRSKSNYYATYYADSVKDRFTISRDDSKNTAYLQMNSLKTEDT | |
| | AVYYCVRGRELGDYYAMDYWGQGTLVTVSS | |
| V_{L} | DIVMTQSPLSLPVTPGEPASISCRSSKSLLHSNGNTYLDWFLQKPGQSP | SEQ ID No: 95 |
| | QLLIYRMSNLASGVPDRFSGSGSGTDFTLKISRVEAEDVGVYYCMQH | |
| | LEYPFTFGQGTKLEIK | |

3F11hz4

| | | |
|---|---|---|
| V_{H} | EVQLVESGGGLVQPGGSLKLSCAASGFSFNTYAMNWVRQASGKGLE | SEQ ID No: 96 |
| | WVGRIRSKSNYYATYYAASVKGRFTISRDDSKSTLYLQMNSLKTEDT | |
| | AVYYCVRGRELGDYYAMDYWGQGTLVTVSS | |
| V_{L} | DIVMTQSPLSLPVTPGEPASISCRSSKSLLHSNGNTYLYWFLQKPGQSP | SEQ ID No: 97 |
| | QLLIYRMSNLASGVPDRFSGSGSGTDFTLKISRVEAEDVGVYYCMQH | |
| | LEYPFTFGQGTKLEIK | |

3F11hz5

| | | |
|---|---|---|
| V_{H} | EVQLVESGGGLVQPGGSLKLSCAASGFSFNTYAMNWVRQASGKGLE | SEQ ID No: 92 |
| | WVGRIRSKSNYYATYYAASVKGRFTISRDDSKNTAYLQMNSLKTEDT | |
| | AVYYCVRGRELGDYYAMDYWGQGTLVTVSS | |
| V_{L} | DIVMTQSPLSLPVTPGEPASISCRSSKSLLHSNGNTYLEWYLQKPGQSP | SEQ ID No: 98 |
| | QLLIYRMSNRASGVPDRFSGSGSGTDFTLKISRVEAEDVGVYYCMQH | |
| | LEYPFTFGQGTKLEIK | |

3F11hz6

| | | |
|---|---|---|
| V_{H} | EVQLVESGGGLVQPGGSLKLSCAASGFSFNTYAMNWVRQASGKGLE | SEQ ID No: 92 |
| | WVGRIRSKSNYYATYYAASVKGRFTISRDDSKNTAYLQMNSLKTEDT | |
| | AVYYCVRGRELGDYYAMDYWGQGTLVTVSS | |
| V_{L} | DIVMTQSPLSLPVTPGEPASISCRSSKSLLHSSGNTYLDWFLQKPGQSP | SEQ ID No: 99 |
| | QLLIYRMSNRASGVPDRFSGSGSGTDFTLKISRVEAEDVGVYYCMQH | |
| | LEYPFTFGQGTKLEIK | |

3O20hz0

| | | |
|---|---|---|
| V_{H} | EVQLVESGGGLVQPKGSLKLSCATSGFSFNAYAMNWVRQAPGKGLE | SEQ ID No: 54 |
| | WVGRIRSKSNNYATYYADSVKGRFTISRDDSKTILYLQMNTLKTEDT | |
| | AIYYCVRGGYHGNSAYFDVWGTGTSVTVSS | |
| V_{L} | DIVMTQAAPSVPVTPGESVSISCRSSKSLLHSNGNTYLYWFLQRPGQS | SEQ ID No: 56 |
| | PQLLIYRMSNLASGVPDRFSGSGSGTAFTLRISRVEAEDVGVYYCLQH | |
| | LEYPFTFGSGTKLEIK | |

3O20hz1

| | | |
|---|---|---|
| V_{H} | EVQLVESGGGLVQPGGSLKLSCAASGFSFNAYAMHWVRQASGKGLE | SEQ ID No: 100 |
| | WVGRIRSKSNNYATAYAASVKGRFTISRDDSKNTAYLQMNSLKTEDT | |
| | AVYYCVRGGYHGNSAYFDVWGQGTLVTVSS | |
| V_{L} | DIVMTQSPLSLPVTPGEPASISCRSSKSLLHSNGNTYLDWYLQKPGQSP | SEQ ID No: 101 |
| | QLLIYRMSNRASGVPDRFSGSGSGTDFTLKISRVEAEDVGVYYCLQHL | |
| | EYPFTFGQGTKLEIK | |

3O20hz2

| | | |
|---|---|---|
| V_{H} | EVQLVESGGGLVQPGGSLKLSCATSGFSFNAYAMNWVRQASGKGLE | SEQ ID No: 102 |
| | WVGRIRSKSNNYATYYAASVKGRFTISRDDSKTTLYLQMNSLKTEDT | |
| | AVYYCVRGGYHGNSAYFDVWGQGTLVTVSS | |
| V_{L} | DIVMTQSPLSLPVTPGEPASISCRSSKSLLHSNGNTYLYWFLQKPGQSP | SEQ ID No: 103 |
| | QLLIYRMSNLASGVPDRFSGSGSGTDFTLKISRVEAEDVGVYYCLQHL | |
| | EYPFTFGQGTKLEIK | |

3O20hz3

| | | |
|---|---|---|
| V_{H} | EVQLVESGGGLVQPGGSLKLSCATSGFSFNAYAMHWVRQASGKGLE | SEQ ID No: 104 |
| | WVGRIRSKSNNYATAYAASVKGRFTISRDDSKTTLYLQMNSLKTEDT | |
| | AVYYCVRGGYHGNSAYFDVWGQGTLVTVSS | |
| V_{L} | DIVMTQSPLSLPVTPGEPASISCRSSKSLLHSQGNTYLDWFLQKPGQSP | SEQ ID No: 105 |
| | QLLIYRMSNRASGVPDRFSGSGSGTDFTLKISRVEAEDVGVYYCLQHL | |
| | EYPFTFGQGTKLEIK | |

1G17hz0

| | | |
|---|---|---|
| V_{H} | QVQLQQSGAELVRPGASVTLSCKASGYTFADYEMHWVKQTPVLGLE | SEQ ID No: 71 |
| | WIGAIDPETGRTAYNQKFKFKATLTADRSSSTAYMELRSLTSEDSAVY | |
| | YCTRRAYWGGWGQGTTLTVSS | |
| V_{L} | DVVMTQTPLSLPVSLGDQASISCRSSQSLVHSSGNTYLHWYLQKPGQ | SEQ ID No: 75 |
| | SPKLLIYKVSNRFSGVPDRFSGSGSGTDFTLKISRVEAEDLGVYFCSQS | |
| | THVPYTFGGGTKLEIK | |

1G17hz1

| | | |
|---|---|---|
| V_{H} | QVQLVQSGAEVKKPGASVKVSCKASGYTFADYYMHWVRQAPGQGL | SEQ ID No: 106 |
| | EWMGAIDPETGRTAYAQKFQGRVTMTRDTSTSTVYMELSSLRSEDT | |
| | AVYYCARRAYWGGWGQGTLVTVSS | |
| V_{L} | DVVMTQSPLSLPVTLGQPASISCRSSQSLVHSSGNTYLHWFQQRPGQS | SEQ ID No: 107 |
| | PRRLIYKVSNRFSGVPDRFSGSGSGTDFTLKISRVEAEDVGVYYCSQS | |
| | THVPYTFGQGTKLEIK | |

1G17hz2

| | | |
|---|---|---|
| V_{H} | QVQLVQSGAEVKKPGASVKVSCKASGYTFADYEMHWVRQAPGQGL | SEQ ID No: 108 |
| | EWIGIIDPETGRTSYAQKFQGRATLTADRSTSTAYMELSSLRSEDTAV | |
| | YYCTRRAYWGGWGQGTLVTVSS | |
| V_{L} | DVVMTQSPLSLPVTLGQPASISCRSSQSLVHSSGNTYLNWYQQRPGQS | SEQ ID No: 109 |
| | PRLLIYKVSNRDSGVPDRFSGSGSGTDFTLKISRVEAEDVGVYFCSQST | |
| | HVPYTFGQGTKLEIK | |

1G17hz3

| | | |
|---|---|---|
| V_{H} | QVQLVQSGAEVKKPGASVKVSCKASGYTFADYEMHWVRQAPGQGL | SEQ ID No: 110 |
| | EWMGAIDPETGRTAYAQKFQGRVTMTADRSTSTAYMELSSLRSEDT | |
| | AVYYCTRRAYWGGWGQGTLVTVSS | |
| V_{L} | DVVMTQSPLSLPVTLGQPASISCRSSQSLVHSSGNTYLHWYQQRPGQS | SEQ ID No: 111 |
| | PRLLIYKVSNRFSGVPDRFSGSGSGTDFTLKISRVEAEDVGVYFCSQST | |
| | HVPYTFGQGTKLEIK | |

All optimized antibodies were confirmed to bind to human CCR8-expressing 293F. The affinity constants for the humanized version of antibodies 3F711, 3O20 and 1G17 are shown in Table 6. The affinity of 3F11hz4, 3O20hz2, 1G17hz3 is similar or better than chimeric antibody such as 3F11hz0, 3O20hz0, 1G17hz0.

TABLE 6
Anti-CCR8 humanized antibody affinity

| Clone | EC₅₀ (nM) |
|---|---|
| 3F11hz0 | 0.3092 |
| 3F11hz1 | N.D.* |
| 3F11hz2 | 98.53 |
| 3F11hz3 | N.D. |
| 3F11hz4 | 0.1938 |
| 3F11hz5 | N.D. |
| 3F11hz6 | N.D. |
| 3O20hz0 | 0.1851 |
| 3O20hz1 | 507.9 |
| 3O20hz2 | 0.1348 |
| 3O20hz3 | 241.1 |
| 1G17hz0 | 0.4511 |
| 1G17hz1 | N.A.** |
| 1G17hz2 | 333.4 |
| 1G17hz3 | 0.4699 |

*N.D. means “not determined”
**N.A. means “not active”

Then 3F11hz4, 3O20hz2, 1G17hz3 were tested if they are cross reactive with mouse CCR8 and cynomolgus CCR8 by FACS like Example 5. The affinity for the humanized antibody 3F11hz4 and 3F11 binding to mouse CCR8 were shown in FIG. 3. After the humanization, the affinity of 3F11hz4 binding to mouse CCR8 were increased from 681.9 nM to 1.926 nM. After the humanization, the affinity of 3O20hz2 and 1G17hz3 binding to cynomolgus CCR8 were 6418 nM and 3142 nM, respectively. 3O20hz2 and 1G17hz3 showed a weak binding affinity with cynomolgus CCR8.

### Example 7. The Calcium Mobilization Assay of Humanized Antibodies

Calcium mobilization assay is a cell-based second messenger assay to measure the calcium flux associated with G-protein coupled receptor activation or inhibition. The change in the fluorescence intensity is directly correlated to the amount of intracellular calcium that is released into cytoplasm in response to ligand activation of the receptor of interest. The assay is used to determine which of the humanized antibodies described above can block the CCL1-CCR8 signal.

The hCCR8-Gqi5-293T cells (constructed by Genomeditech) passed in a complete medium (DMEM medium, 10% FBS, 1% penicillin-streptomycin, 0.75 g/mL puromycin, 400 μg/mL G418) in an incubator (37° C., 5% CO2) were used in the Calcium mobilization assay.

The fluorescent membrane-permeable calcium-binding dye (the FLIPR Calcium 6 Assay Kit) was dissolved in assay buffer (20 mM HEPES buffer with 1* Hank's Balanced Salt Solution (HBSS), pH 7.4). The loading buffer was prepared with the dye solution containing 5 mM probenecid. The probenecid was prepared into 500 mM stock solution in 1 N NaOH, and then diluted to 250 mM in HBSS buffer before use.

Approximately 1.5*10⁴ hCCR8-Gqi5-293T cells were seeded into a 384-well plate and incubated in 25 μL starving medium (DMEM, 1% FBS, 1% penicillin-streptomycin) in 5% CO₂ at 37° C. for 16 hours. Then, the starving medium was completely changed with 25 μL assay buffer, and 25 μL loading buffer was added into the desired wells. After adding dye, the cell plate was incubated for 2 hours at 37° C. with 5% CO₂ and then kept at room temperature until used. The compounds in 12.5 μL assay buffer at desired concentration (5×) were added into each well and incubated with cells for 30 minutes at room temperature. After incubation, the microplate was transferred to the FLIPR instrument and the calcium assay was started as described in the user guide for the instrument. 12.5 μL assay buffer with or without CCL1 was added during the assay. The MAX ratio value was plotted against the antibody concentration and analyzed in GraphPad Prism for concentration curve generation.

All of the humanized antibodies showed a CCL1-CCR8 signaling block and 3F11hz4 showed a strongest CCL1-CCR8 signaling block. The signal blocking ability for the representative antibodies disclosed herein is shown in Table 7.

TABLE 7
Calcium mobilization assay result
of humanized anti-CCR8 antibodies.

| Clone | Calcium mobilization IC₅₀ (nM) |
|---|---|
| 3F11hz4 | 4 |
| 3O20hz2 | 7.39 |
| 1G17hz3 | 25.3 |

### Example 8. Humanized Antibody Stability Validation

Monoclonal antibodies are proteinaceous in nature and are subject to instability issues. Stability testing of monoclonal antibodies is a critical regulatory requirement in their development and commercialization as therapeutic biological molecules. The stability and activity of humanized antibodies such as 3F11hz4, 3O20hz2, 1G17hz3 were tested under the stress condition shown in Table 8.

TABLE 8
Stability test item

| | | Time point and Test Item | |
|---|---|---|---|
| Stress Name | Stress Condition | T0 | Tests |
| T0 | N/A | T0 | Affinity |
| Freeze/thaw | −80° C. to RT | 5FT*** | The Calcium |
| Oxidation | 1% tBHP, 25° C. | 24 h | Mobilization |
| Temperature | 40° C. | 1 W , 2 W , 4 W | Assay |

***5FT refers to 5 cycles of Freeze/Thaw

In a similar manner to Example 2 and 7, the binding affinity and calcium mobilization of humanized antibodies in different time point were performed. The results are shown in Table 9. 3F11hz4 showed better stability than other humanized antibodies.

TABLE 9
The affinity EC50 and calcium mobilization IC50
of humanized antibody in stress condition

| | | | | | Calcium |
|---|---|---|---|---|---|
| | | | | Affinity | mobilization |
| Humanized | Stress | Time | | EC₅₀ (nM) | IC₅₀ (nM) |
| Antibody | condition | point | | Ratio | Ratio |
| 3F11hz4 | T0 | T0 | | 1 | 1 |
| | Freeze/thaw | 5FT | | 1.22 | 1.5 |
| | Oxidation | 24 | h | 1.36 | 1.63 |
| | 40° C. | 1 | W | 1.34 | 1.31 |
| | | 2 | W | 1.27 | 1.03 |
| | | 4 | W | 1.35 | 2.22 |
| 3O20hz2 | T0 | T0 | | 1 | 1 |
| | Freeze/thaw | 5FT | | 1.22 | 1.37 |
| | Oxidation | 24 | h | 2.13 | 1.42 |
| | 40° C. | 1 | W | 0.79 | 3.02 |
| | | 2 | W | 1.14 | 0.98 |
| | | 4 | W | 1.65 | 1.86 |
| 1G17hz3 | T0 | T0 | | 1 | 1 |
| | Freeze/thaw | 5FT | | 2.43 | 0.32 |
| | Oxidation | 24 | h | 1.71 | 1.14 |
| | 40° C. | 1 | W | 0.91 | 3.07 |
| | | 2 | W | 1.31 | 1.38 |
| | | 4 | W | 1.10 | 2.35 |

### Example 9. 3F11hz4 Cross-Reactivity with 293F/CHOK1 Rat, Dog, Mouse and Cynomolgus CCR8

293F cells were transfected with lentiviral vectors encoding rat and dog CCR8 by polybrene (8 g/mL), selected in media containing puromycin (2 μg/mL), and tested for the expression of rat and dog CCR8 by FACS. CHOK1 cells were transfected with lentiviral vectors encoding mouse CCR8 by polybrene (8 μg/mL), selected in media containing puromycin (6 μg/mL), and tested for the expression of mouse CCR8 by FACS.

Humanized antibodies were studied for specific binding to human, rat, dog, mouse and cynomolgus CCR8 by flow cytometry using human, rat, dog, mouse and cynomolgus CCR8-overexpressing and parental 293F cell line or CHOK1 cell line.

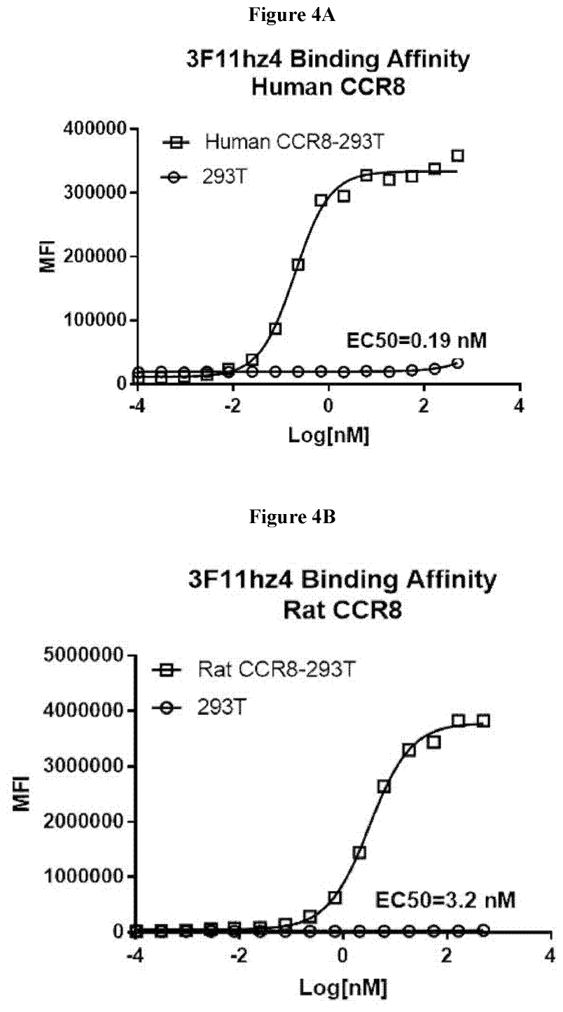

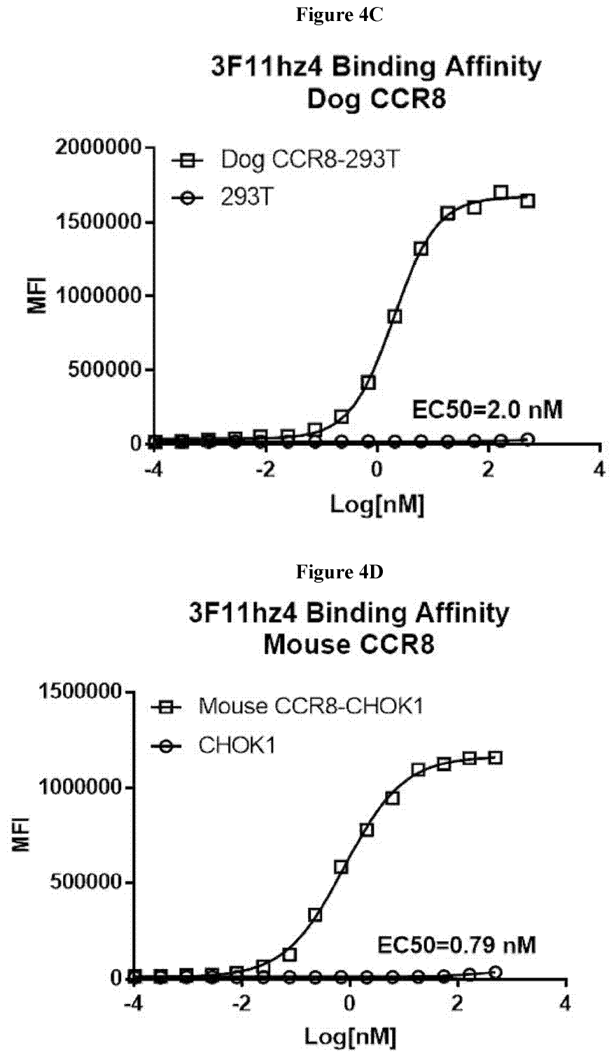

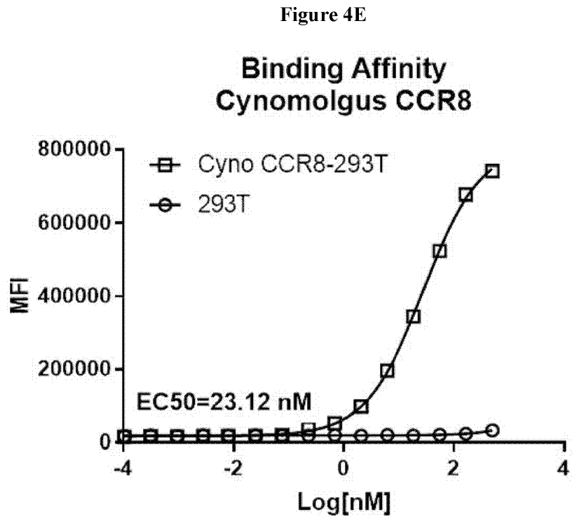

The results are shown in FIG. 4A-4E. According to binding affinity, 3F11hz4 bound HEK293/CHOK1 cells expressing human (4A), rat (4B), dog (4C), mouse (4D) and cynomolgus CCR8 (4E), but not parental cells.

### Example 10. Epitope Analysis of Humanized Monoclonal Antibody

Human, rat, dog, mouse and cynomolgus CCR8 sequence were analysis. The result was shown that ECD2 regions of human, rat, dog, mouse and cynomolgus CCR8 were similar (Table 10). Supposed 3F11hz4 humanized antibody binding to ECD2, the binding evaluation was performed by transiently expressing each mutant of ECD2 in 293F cells, and reacting the mutant with an antibody solution of a humanized 3F11hz4 antibody prepared by 14 serially diluting by 3-fold from 25 μg/mL. After reacting at 4° C. for 1 hour, it was reacted with Alexa Fluor 488 affinpure goat anti-human Ig(H+L) (Jackson, 109-545-003) and flow cytometry analysis was performed. The highest MFI of serially dilution is supposed as 100%, the inhibition percent was calculated according to the following formula.

Inhibition %=(Highest MFI−MFI)/Highest MFI*100%

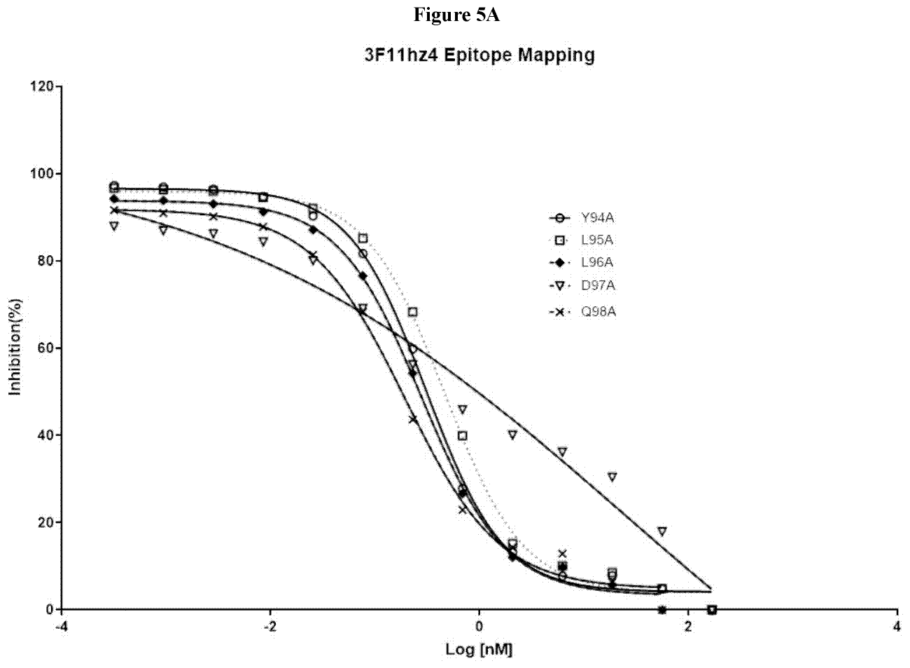

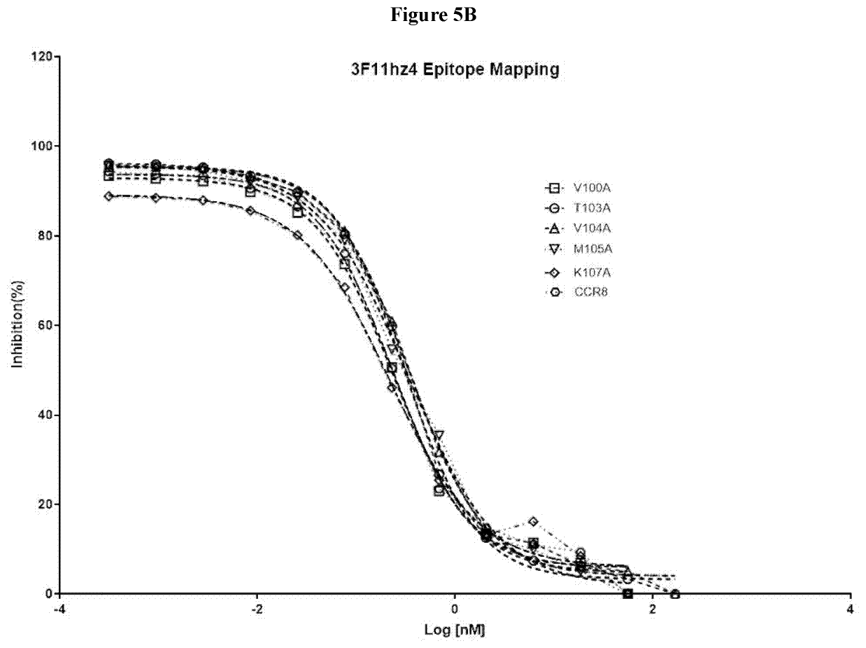

The results are shown in FIG. 5A-B and Table 11. In comparison to human CCR8, a mutant of 97 aa in CCR8 can rise the EC50 from 0.2335 nM to 23.96 nM.

TABLE 10
CCR8 gene homology

| | Human | Human | Human | Human |
|---|---|---|---|---|
| CCR8 | vs. rat | vs. dog | vs. mouse | vs. cynomolgus |
| ECD1 | 59% | 71% | 60% | 77% |
| ECD2 | 100% | 93% | 92% | 93% |
| ECD3 | 59% | 84% | 65% | 97% |
| ECD4 | 60% | 67% | 60% | 94% |

TABLE 11
Binding activity of 3F11hz4 in mutant CCR8

| Mutant | Inhibition EC50 (nM) |
|---|---|
| hCCR8(Y94A) | 0.3064 |
| hCCR8(L95A) | 0.4588 |
| hCCR8(L96A) | 0.2796 |
| hCCR8(D97A) | 23.96 |
| hCCR8(Q98A) | 0.194 |
| hCCR8(V100A) | 0.2368 |
| hCCR8(T103A) | 0.3114 |
| hCCR8(V104A) | 0.3414 |
| hCCR8(M105A) | 0.3338 |
| hCCR8(K107A) | 0.22 |
| hCCR8 | 0.2335 |

### Example 11. Study of 3F11 hz4 Humanized Antibody for Binding to Human CCR4 and Human CX3CR1

3F11hz4 humanized antibody was tested for binding to CCR4 and CX3CR1 overexpressing cells by flow cytometry. Briefly, 293F cells were transfected with lentiviral vectors encoding human CCR4 and CX3CR1 by polybrene (8 μg/mL), selected in media containing puromycin (2 μg/mL), and tested for the expression of human CCR4 and CX3CR1 by FACS using CCR4 (Biolegend, 359408) and CX3CR1 antibody (Biolegend, 341610). The binding evaluation was performed by expressing human CCR4 and CXCR1 in 293F cells, and reacting the cells with an antibody solution of a humanized 3F11hz4 antibody prepared by 14 serially diluting by 3-fold from 25 μg/mL. After reacting at 4° C. for 1 hour, it was reacted with Alexa Fluor 488 affinipure goat anti-human Ig(H+L) (Jackson, 109-545-003) and flow cytometry analysis was performed.

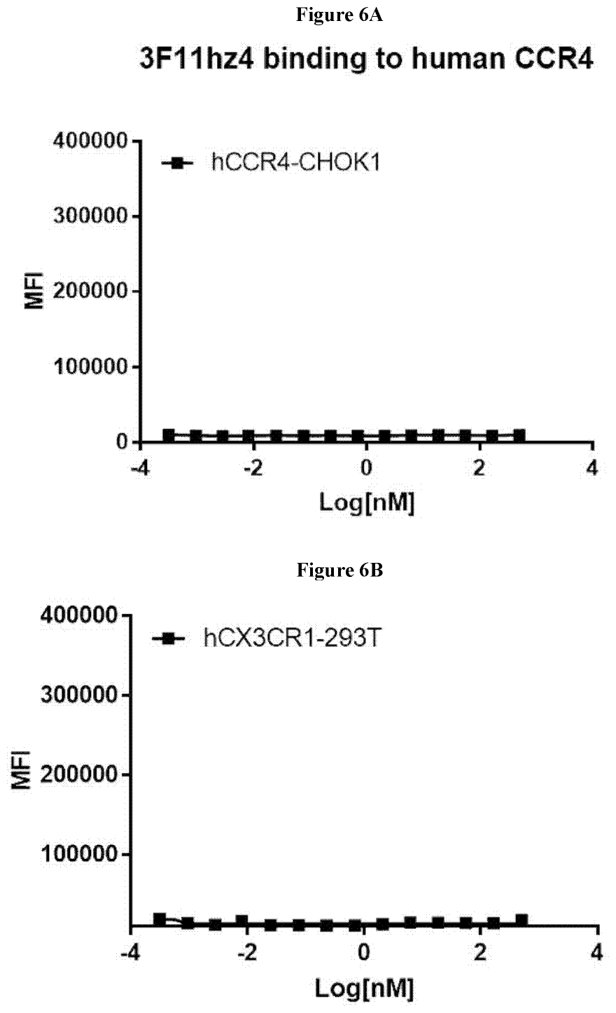

As shown in FIG. 6A-6B, humanized 3F11hz4 antibody bound 293F cells expressing human CCR8(4A), but did not bind 293F cells expressing human CCR4(6A), human CX3CR1(6B), or 293F cells (4A).

### Example 12. 3F11hz4 Humanized Antibody Compared with Other CCR8 Antibodies

The binding affinity, species Cross reactivity, and signaling block of the antibody disclosed herein 3F11hz4 humanized antibody and other prior art CCR8 antibodies were studies. The prior art CCR8 antibodies were produced and characterized according to the relevant references. Specifically, the CCR8 antibody sequences of Gilead (WO2021163064), Shionogi (EP3903817A1), Surface (SRF-114) and Bayer (TPP-23411, WO2021152186) were expressed through CHO cells. The various antibodies were compared through the affinity (EC50), species cross reactive and calcium mobilization (IC50). The results were shown in Table 12.

TABLE 12
3F11hz4 current competitor comparison results

| | | | Binding | | |
|---|---|---|---|---|---|
| | | | affinity | Sepcies | Signal block |
| Product | Organization | Phase | EC₅₀ (nM) | Cross | Ca2+ (nM) |
| S-531011 | Shionogi | Phase I/II | 0.2434 | Dog/cyno | Yes |
| GS-1811 | Gilead | Phase I | 0.29 | None | 4.343 ± 2.589 |
| SRF-114 | Surface/Vaccinex | Preclinical | 3.2 | None | 179 |
| TPP-23411 | Bayer | Preclinical | 0.65 | Cyno | Not active |
| 3F11hz4 | Immunophage | Preclinical | 0.19 ± 0.035 | Dog/mouse/ | 5.673 ± 1.597 |
| | | | | rat/cyno | |

As shown in Table 12, 3F11 hz4 showed the advantages in the binding affinity, species cross reactivity and signal block Ca²⁺.

### Example 13. Anti-Tumor Efficacy of 3F11hz4 Antibody in CD34 Humanized Mice

Preparation and detection of hu-HSC-NPG human immune system mouse model: 4-week-old FEMALE NPG mice were irradiated by X-ray biological irradiator for 4-24 h and injected with CD34+ hematopoietic stem cells derived from umbilical cord blood through tail vein. Sixteen weeks after transplantation, blood samples were collected through orbit, and blood samples were collected by EDTA-Na₂ anticoagulation tube. Flow cytometry was used to analyze the content of human CD45, CD3, CD4, and CD8 positive cells in peripheral blood of animals to determine the proportion of human T cells implanted Hu-HSC-NPG with high T cell implantation ratio was selected to establish the tumor bearing model.

Tumor inoculation: Lung cancer (HCC827) were cultured and expanded to a sufficient number, suspended in PBS solution, and injected into the dorsal subcutaneous region at a dose of 5×10⁶ cells/0.2 ml. A total of 45 mice were inoculated in each round of the experiment and the volume was observed when the subcutaneous tumor was clearly visible. After 7-14 days, the subcutaneous tumors grew to 50-100 mm³. Some mice with too large or too small subcutaneous tumors were removed and randomly divided into 4 groups with 10 mice in each group. Categories as follows: Group1: IgG1 10 mpk, iv, biw; Group2: CCR8-Ab-IgG1 1 mpk, iv, biw; Group3: CCR8-Ab-IgG1 3 mpk, iv, biw; Group4: CCR8-Ab-IgG1 10 mpk, iv, biw. Animals were injected with CCR8 antibody intravenously (IV.). The antibody was administered twice a week for a total of 5-6 times, depending on the rate of growth of the subcutaneous tumor. The volume of subcutaneous tumors and the weight of mice were measured twice a week. Tumor volume calculation formula: volume=long diameter×short diameter×short diameter/2.

Statistical methods: Data analysis GraphPad Prism 8.0 statistical analysis software was used to analyze whether there was statistical difference in subcutaneous tumor volume between CCR8 antibody treatment group and isotype control group. First, the data were tested for normal distribution and homogeneity of variance. It was in line with normal distribution (P>0.20) and homogeneity of variance (P>0.10): Comparison between multiple groups was tested by one-way ANOVA method, and P<0.05 was considered statistically significant; The kruskal-Wallis H method of non-parametric test was used for analysis if it did not conform to normal distribution or variance. Tumor volume inhibition rate (TGI) was calculated as follows: TGI=100%×[1−RTV(experimental group)/RTV(control group)].

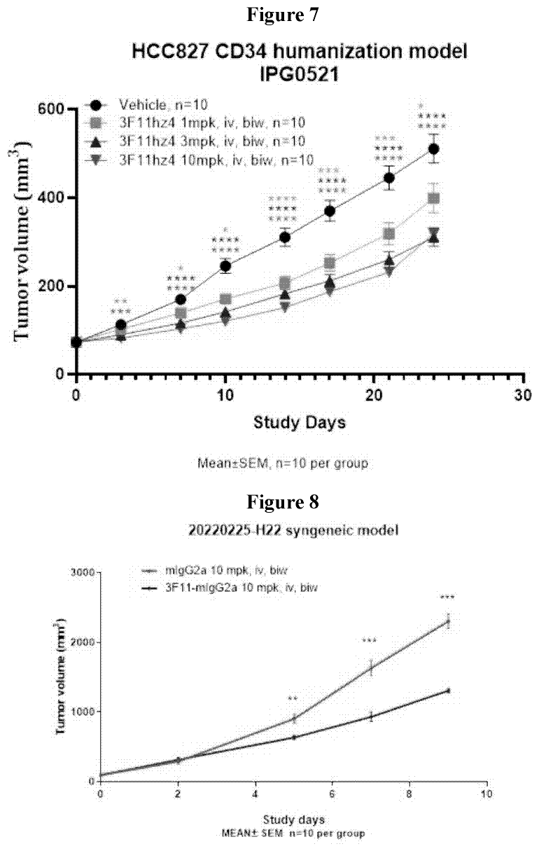

Conclusion: The dose-dependent anti-tumor effect of 3F11hz4 antibody on subcutaneous lung cancer (HCC827 cell line) has been confirmed in CD34 humanized animal models (FIG. 7).

### Example 14. Anti-Tumor Efficacy of 3F11hz4 Antibody in Syngeneic Model

In solid tumor models such as lung cancer and liver cancer, the dose-effect relationship of 3F11hz4 was investigated to explore the lowest effective dose and maximum effective dose.

In vivo efficacy verification of syngeneic transplanted tumor models of lung and liver cancer in mice: 50 8-weeks-old female mice were prepared for each round of the experiment. Lung cancer (LLC) and liver cancer (H22) of 1×10⁶ mice were inoculated in 200 μL PBS subcutaneously on the right side of the abdomen of the mice and the inoculation date was recorded as day 0. After 4-7 days, the subcutaneous tumors grew to 50-100 mm³. According to the volume of subcutaneous tumors, some mice with too large or too small subcutaneous tumors were removed and randomly divided into 4 groups with 10 mice in each group. Categories as follows: Group1: mIgG2a 10 mpk, ip, biw; Group2: CCR8-Ab-mIgG2a 1 mpk, ip, biw; Group3: CCR8-Ab-mIgG2a 3 mpk, ip, biw; Group4: CCR8-Ab-mIgG2a 10 mpk, ip, biw. Intraperitoneal CCR8 antibody (I. P.) Injected animals. The antibody is administered twice a week for a total of 3-6 times, depending on the rate of growth of the subcutaneous tumor. The volume of subcutaneous tumors and the weight of mice were measured twice a week. Tumor volume calculation formula: volume=long diameter×short diameter×short diameter/2. GraphPad software was used to analyze whether there was statistical difference in subcutaneous tumor volume between CCR8 antibody treatment group and homotype control group.

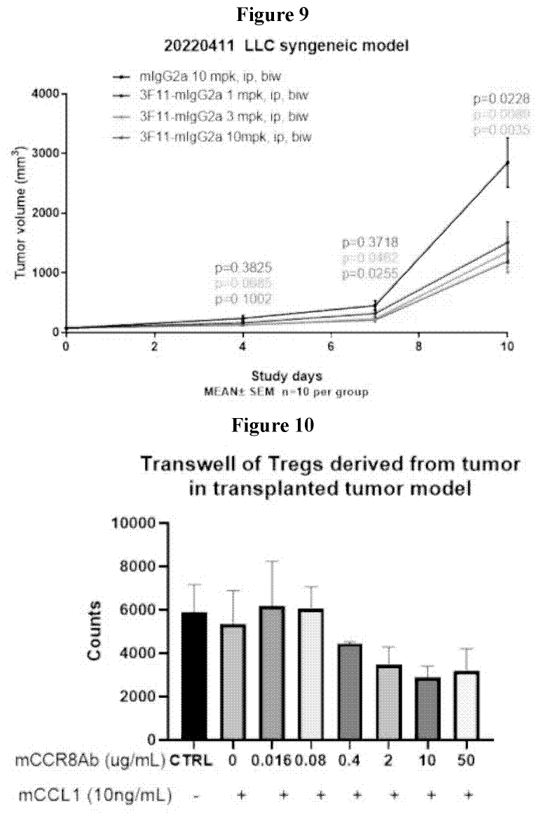

Conclusion: The dose-dependent anti-tumor effect of 3F11hz4 antibody on subcutaneous lung cancer (HCC827 cell line) has been confirmed in CD34 humanized animal models (FIG. 7) and in mouse hepatoma (H22 cell line) and syngeneic mouse lung cancer (LLC cell line) models (FIGS. 8 and 9).

### Example 15. Treg Cell Migration Test

Treg cell migration test: CD4+CD25+Treg cells in mouse H22 subcutaneous tumor were enriched by magnetic bead sorting method and CCR8 antibody flow cytometry was used to detect the CCR8 expression rate of the enriched Treg cells, and the migration test was performed when the CCR8 expression rate was more than 80%. CD4+CD25+Treg cells were treated with starvation medium 1640+1% FBS (Ce)+1% P/S for 3 h. Cells were collected, centrifugally counted, and suspended to 2.66×10⁶/mL in 1640+0.5% BSA medium. Dilution of CCR8 antibody with migration medium 1640+0.5% BSA+1% P/S: First, add 2 μL 10 mg/mL CCR8 antibody to 998 μL 1640+0.5% BSA+1% P/S medium and dilute to 50 μg/mL; A positive control group without CCR8 antibody was set according to the gradient dilution of 6 sites at 5 times. The 75 μL diluted CCR8 antibody was mixed with 75 μL cell suspension, placed in a 37° C. incubator, and incubated for 30 min. CCL1 was diluted with migration medium 1640+0.5% BSA+1% P/S (1 mL per 8 Wells). 100 μL 10 ng/mL CCL1 diluent was pre-added to the lower layer of Transwell plate. Add 75 μL cells co-incubated with CCR8 antibody to the upper layer of Transwell plate. Culture in 37° C. incubator for 3 hours. The number of lower cells was counted by flow cytometry, and the high speed was set to 60 ul/min and 1 min. GraphPad Prism processed data to analyze the inhibition degree of CCR8 antibody on Treg cell migration.

Conclusion: Tregs cells selected from subcutaneous tumor tissues of H22 liver cancer were used to verify that CCR8 antibody could inhibit migration of Tregs cells (FIG. 10).

All the documents mentioned in the present invention are incorporated in the present application by reference to the same extent as if each individual document is specifically and individually indicated to be incorporated by reference. In addition, it should be understood that after reading the contents taught in the present invention, various modifications and changes may be made to the present invention by those skilled in the art, and these equivalents also fall into the scope defined by the claims.

What is claimed:

1. An antibody or an antigen-binding fragment thereof which comprises at least one heavy chain variable region comprising a HCDR1, a HCDR2 and a HCDR3 selected from the group consisting of:
a) SEQ ID NOs: 58, 79, and 80; or
b) SEQ ID NOs: 2, 3, and 4; or
c) SEQ ID NOs: 2, 3, and 10; or
d) SEQ ID NOs: 2, 16, and 17; or
e) SEQ ID NOs: 22, 23, and 24; or
f) SEQ ID NOs: 32, 33, and 34; or
g) SEQ ID NOs: 40, 41, and 42; or
h) SEQ ID NOs: 47, 48, and 49; or
i) SEQ ID NOs: 40, 3, and 55; or
j) SEQ ID NOs: 58, 59, and 60; or
k) SEQ ID NOs: 58, 3, and 63; or
l) SEQ ID NOs: 2, 3, and 67; or
m) SEQ ID NOs: 72, 73, and 74; or
n) SEQ ID NOs: 58, 83, and 84; or
o) SEQ ID NOs: 58, 83, and 88; or
and comprising at least one light chain variable region comprising a LCDR1, a LCDR2 and a LCDR3 selected from the group consisting of:
a) SEQ ID NOs: 6, 7, and 14; or
b) SEQ ID NOs: 6, 7, and 8; or
c) SEQ ID NOs: 12, 7, and 14; or
d) SEQ ID NOs: 19, 7, and 14; or
e) SEQ ID NOs: 26, 20, and 27; or
f) SEQ ID NOs: 30, 20, and 27; or
g) SEQ ID NOs: 36, 37, and 38; or
h) SEQ ID NOs: 44, 37, and 45; or
i) SEQ ID NOs: 51, 52, and 53; or
j) SEQ ID NOs: 6, 7, and 8; or
k) SEQ ID NOs: 65, 7, and 14; or
l) SEQ ID NOs: 69, 70, and 13; or
m) SEQ ID NOs: 76, 37, and 77; or
n) SEQ ID NOs: 86, 7, and 14.
wherein the antibody or an antigen-binding fragment thereof specifically binds CCR8; and any of the above amino acid sequences further includes a derivative sequence formed by optionally addition, deletion, modification, and/or substitution of 1-5 (or 1, 2, 3) amino acids, and capable of retaining CCR8 binding ability.

2. The antibody or an antigen-binding fragment thereof of claim 1, wherein said antibody or an antigen-binding fragment thereof comprising a HCDR1, a HCDR2, a HCDR3, a LCDR1, a LCDR2 and a LCDR3 selected from the group consisting of:
a) SEQ ID NOs: 58, 79, 80, 6, 7, and 14; or
b) SEQ ID NOs: 2, 3, 4, 6, 7, and 8; or
c) SEQ ID NOs: 2, 3, 10, 12, 7, and 14; or
d) SEQ ID NOs: 2, 16, 17, 19, 7, and 14; or
e) SEQ ID NOs: 22, 23, 24, 26, 20, and 27; or
f) SEQ ID NOs: 22, 23, 24, 30, 20, and 27; or
g) SEQ ID NOs: 32, 33, 34, 36, 37, and 38; or
h) SEQ ID NOs: 40, 41, 42, 44, 37, and 45; or
i) SEQ ID NOs: 47, 48, 49, 51, 52, and 53; or
j) SEQ ID NOs: 40, 3, 55, 6, 7, and 8; or
k) SEQ ID NOs: 58, 59, 60, 6, 7, and 14; or
l) SEQ ID NOs: 58, 3, 63, 65, 7, and 14; or
m) SEQ ID NOs: 2, 3, 67, 69, 70, and 13; or
n) SEQ ID NOs: 72, 73, 74, 76, 37, and 77; or
o) SEQ ID NOs: 58, 83, 84, 86, 7, and 14; or
p) SEQ ID NOs: 58, 83, 88, 86, 7, and 14.

3. The antibody of claim 1 or 2, wherein the heavy chain further includes a heavy chain constant region and/or the light chain further includes a light chain constant region.

4. The antibody of claim 1 or 2, wherein the heavy chain variable region of the antibody further comprises a human or humanized framework region, and/or the light chain variable region of the antibody further comprises a human or humanized framework region.

5. The antibody of claim 1 or 2, wherein the polypeptide sequences are selected from the group consisting of:
a) a heavy chain variable region having the polypeptide sequence of SEQ ID NO: 96, and a light chain variable region having the polypeptide sequence of SEQ ID NO: 97; or
b) a heavy chain variable region having the polypeptide sequence of SEQ ID NO: 78, and a light chain variable region having the polypeptide sequence of SEQ ID NO: 81; or
c) a heavy chain variable region having the polypeptide sequence of SEQ ID NO: 1, and a light chain variable region having the polypeptide sequence of SEQ ID NO: 5; or
d) a heavy chain variable region having the polypeptide sequence of SEQ ID NO: 9, and a light chain variable region having the polypeptide sequence of SEQ ID NO: 11; or
e) a heavy chain variable region having the polypeptide sequence of SEQ ID NO: 15, and a light chain variable region having the polypeptide sequence of SEQ ID NO: 18; or
f) a heavy chain variable region having the polypeptide sequence of SEQ ID NO: 21, and a light chain variable region having the polypeptide sequence of SEQ ID NO: 25; or
g) a heavy chain variable region having the polypeptide sequence of SEQ ID NO: 28, and a light chain variable region having the polypeptide sequence of SEQ ID NO: 29; or
h) a heavy chain variable region having the polypeptide sequence of SEQ ID NO: 31, and a light chain variable region having the polypeptide sequence of SEQ ID NO: 35; or
i) a heavy chain variable region having the polypeptide sequence of SEQ ID NO: 39, and a light chain variable region having the polypeptide sequence of SEQ ID NO: 43; or
j) a heavy chain variable region having the polypeptide sequence of SEQ ID NO: 46, and a light chain variable region having the polypeptide sequence of SEQ ID NO: 50; or
k) a heavy chain variable region having the polypeptide sequence of SEQ ID NO: 54, and a light chain variable region having the polypeptide sequence of SEQ ID NO: 56; or
l) a heavy chain variable region having the polypeptide sequence of SEQ ID NO: 57, and a light chain variable region having the polypeptide sequence of SEQ ID NO: 61; or
m) a heavy chain variable region having the polypeptide sequence of SEQ ID NO: 62, and a light chain variable region having the polypeptide sequence of SEQ ID NO: 64; or
n) a heavy chain variable region having the polypeptide sequence of SEQ ID NO: 66, and a light chain variable region having the polypeptide sequence of SEQ ID NO: 68; or
o) a heavy chain variable region having the polypeptide sequence of SEQ ID NO: 71, and a light chain variable region having the polypeptide sequence of SEQ ID NO: 75; or
p) a heavy chain variable region having the polypeptide sequence of SEQ ID NO: 82, and a light chain variable region having the polypeptide sequence of SEQ ID NO: 85; or
q) a heavy chain variable region having the polypeptide sequence of SEQ ID NO: 87, and a light chain variable region having the polypeptide sequence of SEQ ID NO: 89; or
r) a heavy chain variable region having the polypeptide sequence of SEQ ID NO: 90, and a light chain variable region having the polypeptide sequence of SEQ ID NO: 91; or
s) a heavy chain variable region having the polypeptide sequence of SEQ ID NO: 92, and a light chain variable region having the polypeptide sequence of SEQ ID NO: 93; or
t) a heavy chain variable region having the polypeptide sequence of SEQ ID NO: 94, and a light chain variable region having the polypeptide sequence of SEQ ID NO: 95; or
u) a heavy chain variable region having the polypeptide sequence of SEQ ID NO: 92, and a light chain variable region having the polypeptide sequence of SEQ ID NO: 98; or
v) a heavy chain variable region having the polypeptide sequence of SEQ ID NO: 92, and a light chain variable region having the polypeptide sequence of SEQ ID NO: 99; or
w) a heavy chain variable region having the polypeptide sequence of SEQ ID NO: 100, and a light chain variable region having the polypeptide sequence of SEQ ID NO: 101; or
x) a heavy chain variable region having the polypeptide sequence of SEQ ID NO: 102, and a light chain variable region having the polypeptide sequence of SEQ ID NO: 103; or
y) a heavy chain variable region having the polypeptide sequence of SEQ ID NO: 104, and a light chain variable region having the polypeptide sequence of SEQ ID NO: 105; or
z) a heavy chain variable region having the polypeptide sequence of SEQ ID NO: 106, and a light chain variable region having the polypeptide sequence of SEQ ID NO: 107; or
aa) a heavy chain variable region having the polypeptide sequence of SEQ ID NO: 108, and a light chain variable region having the polypeptide sequence of SEQ ID NO: 109; or
bb) a heavy chain variable region having the polypeptide sequence of SEQ ID NO: 110, and a light chain variable region having the polypeptide sequence of SEQ ID NO: 111.

6. The antibody of any one of claims 1-5, wherein the antibody or an antigen-binding fragment thereof comprises a heavy chain variable region having a polypeptide sequence at least (≥) 85%, at least 90%, at least 95%, at least 96%, at least 97%, at least 98%, or at least 99% identical to SEQ ID NO: 1, 9, 15, 21, 28, 31, 39, 46, 54, 57, 62, 66, 71, 78, 82, 87, 90, 92, 94, 96, 100, 102, 104, 106, 108, or 110, or a light chain variable region having a polypeptide sequence at least 85%, at least 90%, at least 95%, at least 96%, at least 97%, at least 98%, or at least 99% identical to SEQ ID NO: 5, 11, 18, 25, 29, 35, 43, 50, 56, 61, 64, 68, 75, 81, 85, 89, 91, 93, 95, 97, 98, 99, 101, 103, 105, 107, 109, or 111.

7. The antibody of any one of claims 1-6, wherein the antibody is a monoclonal antibody, a recombinant antibody, a human antibody, a humanized antibody, a chimeric antibody, a bispecific antibody, a multispecific antibody, or an antibody fragment thereof.

8. The antibody of any of claim 7, wherein the antibody is selected from the group consisting of: (i) a single chain antibody, a single-chain variable fragment (scFv), a univalent antibody lacking a hinge region or a minibody; (ii) a Fab, Fab′ or F(ab′)₂ fragment; (iii) a whole antibody; and (iv) an antibody that comprises a human IgG Fc domain.

9. The antibody of claim 7, wherein the antibody is human or humanized.

10. The antibody or an antigen-binding fragment thereof of claim 1 or 2, wherein the antibody or an antigen-binding fragment thereof is an CCR8 specific antibody capable of binding human CCR8.

11. The antibody or an antigen-binding fragment thereof of claim 10, wherein the antibody or an antigen-binding fragment thereof is an CCR8 specific antibody capable of binding human CCR8 and blocking CCL1-CCR8 signaling.

12. An antibody or an antigen-binding fragment thereof which specifically binds to CCR8 at an epitope comprising one or more amino acid residues selected from the group consisting of Y94 to K107 of human CCR8.

13. The antibody or an antigen-binding fragment thereof of claim 12, which specifically binds to human CCR8 at an epitope comprising one or more amino acid residues selected from the group consisting of Y94, L95, L96, D97, Q98, V100, T103, V104, M105, and K107 of human CCR8.

14. The antibody or an antigen-binding fragment thereof of claim 13, which specifically binds to human CCR8 at an epitope comprising one amino acid residue selected from the group consisting of Y94, L95, L96, Q98, V100, T103, V104, M105, and K107 of human CCR8.

15. A recombinant protein which comprises:
(i) the antibody or an antigen-binding fragment thereof of any of claims 1-14; and
(ii) optional tag sequences to assist expression and/or purification.

16. An isolated nucleic acids molecule encoding the antibodies or antigen-binding fragments any of claims 1-14, or the recombinant protein of claim 15.

17. A vector which comprises the isolated nucleic acids molecule according to claim 16.

18. The vector of claim 17, which comprises bacterial plasmids, phages, yeast plasmid, plant cell virus, mammalian cell virus such as adenovirus, lentivirus, or retrovirus.

19. An engineered host cell, which comprises the nucleic acids of claim 16 in its genome, or comprises a vector of claim 17.

20. An isolated polynucleotide sufficient for use as a hybridization probe, PCR primer or sequencing primer that is a fragment of the nucleic acid molecule of claim 16 or its complement.

21. An antibody conjugate, which comprises:
(i) an antibody moiety selected from the group consisting of an antibody or antigen-binding fragment of any of claims 1-14, or a recombinant protein of claim 15, or a combination thereof; and
(ii) a coupling moiety coupled to the antibody moiety, wherein the coupling moiety is selected from the group consisting of a detectable label, a drug, a toxin, a cytokine, a radionuclide, an enzyme, or a combination thereof.

22. A pharmaceutical composition, which comprises (i) the antibody or an antigen-binding fragment thereof of any of claims 1-14, the recombinant protein of claim 15, the isolated nucleic acid molecule of claim 16, the vector of claim 17, the antibody conjugate of claim 21, or combinations thereof, and (ii) a pharmaceutically acceptable carrier.

23. The pharmaceutical composition of claim 22, wherein the antibody against CCR8 has an effect of removing tumor-infiltrating Tre, cells.

24. A method for treating a disease mediated by CCR8 and/or CCL1, which comprises administering an effective amount of the antibody or an antigen-binding fragment thereof of any of claims 1-14, the recombinant protein of claim 15, the isolated nucleic acid molecule of claim 16, the vector of claim 17, the antibody conjugate of claim 21, or the pharmaceutical composition of claim 22, to a subject in need.

25. The method of claim 24, wherein the disease is cancer.

26. The method of claim 25, wherein the disease is breast cancer, gastric cancer, ovarian cancer, pancreatic cancer, liver cancer, colon cancer, or pancreatic cancer.

27. The method of claim 24, wherein the disease is neuropathic pain.

28. The method of claim 27, wherein the neuropathic pain induced by diabetes or spinal cord injury.

29. The method of claim 24, wherein the disease is IgG4-related disease.

30. The method of claim 26, wherein the IgG4-related disease is IgG4-related sclerosing cholangitis.

31. A method of determining a level of CCR8 in a subject which comprise (a) obtaining a sample from the subject; (b) contacting the sample with antibody or an antigen-binding fragment thereof of any of claims 1-9; and (c) determining a level of CCR8 in the subject.

32. A use of an active ingredient for (a) preparation of a diagnostic reagent or kit; and/or (b) preparation of a medicament for the prevention and/or treatment of a disease associated with CCR8, wherein the active ingredient is selected from the group consisting of the antibody or an antigen-binding fragment thereof of any of claims 1-14, the recombinant protein of claim 15, the isolated nucleic acid molecule of claim 16, the vector of claim 17, the antibody conjugate of claim 20, and combinations thereof.

33. The use of claim 32 wherein the disease is cancer.

34. The use of claim 33, wherein the disease is breast cancer, gastric cancer, ovarian cancer, pancreatic cancer, liver cancer, colon cancer, or pancreatic cancer.

35. A detection kit which comprises:
(i) a first container containing the antibody of any of claims 1-14 as a first antibody; and
(ii) a second container containing a secondary antibody against the first antibody.

36. A method for preparing a recombinant polypeptide according to any of claim 1-10, which comprises:
(i) culturing an engineered host cell under conditions suitable for expression; and
(ii) isolating a recombinant polypeptide from the culture, wherein the recombinant polypeptide is an antibody, an antigen-binding fragment thereof, or a recombinant protein.
